(19) United States
(12) Patent Application Publication
(10) Pub. No.: US 2004/0058325 A1
(43) Pub. Date: Mar. 25, 2004

(54) Gene expression in biological conditions

(76) Inventors: Torben F Orntoft, Aabyhoi (DK); Thomas Thykjaer, Brabrand (DK); Karin Demtroder, Ronde (DK); Casper Moller Frederiksen, Copenhagen (DK)

Correspondence Address: BANNER & WITCOFF, 1001 G STREET N W, SUITE 1100, WASHINGTON, DC, 20001, US

(21) Appl. No.: 10/169,113

(22) PCT Filed: Dec. 29, 2000
(86) PCT No.: PCT/DK00/00744

(30) Foreign Application Priority Data
Dec. 29, 1999 (DK) PA 1999 01867

(51) Int. Cl. C12Q 1/68; C07H 21/04

(52) U.S. Cl. 435/6; 536/24.3

(57) ABSTRACT

The invention concerns a method of determining the presence or absence of a biological condition in humans, in particular of colon cancer, and of determining the stage of a condition in human tissue by determining an expression pattern of a cell sample. Further, the invention relates to a method of determining the presence or absence of a biological condition in human tissue, and of determining the stage of a biological condition in human tissue, and also for reducing biological abnormalities of a cell suffering from the biological condition. A method for producing antibodies against an expression product of a cell from the tissue is also described. The invention also discloses a pharmaceutical composition for the treatment of a biological condition comprising at least one antibody, and a vaccine for the prophylaxis or treatment of a biological condition. Further the invention describes the use of a method for producing an assay for diagnosing a biological condition in human tissue, the use of a peptide or a gene or a probe for the preparation of a pharmaceutical composition for the treatment of a biological condition in human tissue, and an assay for determining the presence or absence of biological condition in human tissue and for determining an expression pattern of a cell.

### TECHNICAL FIELD OF THE INVENTION

The present invention relates to method of determining the presence or absence of a biological condition in animal tissue, wherein the expression of genes in normal tissue and tissue from the biological condition is examined and correlated to standards. The invention further relates to treatment of the biological condition and an assay for determining the condition.

### BACKGROUND

The building of large databases containing human genome sequences is the basis for studies of gene expressions in various tissues during normal physiological and pathologic conditions. Constantly (constitutively) expressed sequences as well as sequences whose expression is altered during disease processes are important for our understanding of cellular properties, and for the identification of candidate genes for future therapeutic intervention. As the number of known genes and ESTs build up in the databases, array-based simultaneous screening of thousands of genes is necessary to obtain a profile of transcriptional behaviour, and to identify key genes that either alone or in combination with other genes, control various aspects of cellular life. One cellular behaviour that has been a mystery for many years is the malignant behaviour of cancer cells. We now know that for example defects in DNA repair can lead to cancer but the cancer-creating mechanism in heterozygous individuals is still largely unknown as is the malignant cell's ability to repeat cell cycles to avoid apoptosis to escape the immune system to invade and metastasize and to escape therapy. There are hints and indications in these areas and excellent progress has been made, buth the myriad of genes interacting with each other in a highly complex multidimensional network is making the road to insight long and contorted.

Similar appearing tumors—morphologically, histochemically, microscopically—can be profoundly different. They can hav a different invasive and metastasizing properties, as well as respond differently to therapy. There is thus a need in the art for methods which distinguish tumors and tissues on different bases than are currently in use in the clinic.

The malignant transformation from normal tissue to cancer is believed to be a multistep process, in which tumorsuppressor genes, that normally repress cancer growth show reduced gene expression and in which other genes that encode tumor promoting proteins (oncogenes) show an increased expression level. Several tumor suppressor genes have been identified up till now, as e.g. p16, Rb, p53 (Nesrin Özören and Wafik S. El-Deiry, Introduction to cancer genes and growth control, In: DNA alterations in cancer, genetic and epigenetic changes, Eaton publishing, Melanie Ehrlich (ed) p. 1-43, 2000.; and references therein). They are usually identified by their lack of expression or their mutation in cancer tissue.

Other examinations have shown this downregulation of transcripts to be partly due to loss of genomic material (loss of heterozygosity), partly to methylation of promotorregions, and partly due to unknown factors (Nesrin Özören and Wafik S. El-Deiry, Introduction to cancer genes and growth control, In: DNA alterations in cancer, genetic and epigenetic changes, Eaton publishing, Melanie Ehrlich (ed) p. 1-43, 2000.; and references therein).

Several oncogenes are known, e.g. cyclinD1/PRAD1/BCL1, FGFs, c-MYC, BCL-2 all of which are genes that are amplified in cancer showing an increased level of transcript (Nesrin Özören and Wafik S. El-Deiry, Introduction to cancer genes and growth control, In: DNA alterations in cancer, genetic and epigenetic changes, Eaton publishing, Melanie Ehrlich (ed) p. 1-43, 2000.; and references therein). Many of these genes are related to cell growth and directs the tumor cells to uninhibited growth. Others may be related to tissue degradation as they e.g. encode enzymes that break down the surrounding connective tissue.

### SUMMARY OF THE INVENTION

In one aspect the present invention relates to a method of determining the presence or absence of a biological condition in animal tissue comprising

collecting a sample comprising cells from the tissue and/or expression products from the cells,

assaying a first expression level of at least one gene from a first gene group, wherein the gene from the first gene group is selected from genes expressed in normal tissue cells in an amount higher than expression in biological condition cells, and/or

assaying a second expression level of at least one gene from a second gene group, wherein the second gene group is selected from genes expressed in a normal tissue cells in an amount lower than expression in biological condition cells,

correlating the first expression level to a standard expression level for normal tissue, and/or the second expression level to a standard expression level for biological condition cells to determine the presence or absence of a biological condition in the animal tissue.

Animal tissue may be tissue from any animal, preferably from a mammal, such as a horse, a cow, a dog, a cat, and more preferably the tissue is human tissue. The biological condition may be any condition exhibiting gene expression different from normal tissue. In particular the biological condition relates to a malignant or premalignant condition, such as a tumor or cancer.

Furthermore, the invention relates to a method of determining the stage of a biological condition in animal tissue,

comprising collecting a sample comprising cells from the tissue,

assaying the expression of at least a first stage gene from a first stage gene group and at least a second stage gene from a second stage gene group, wherein at least one of said genes is expressed in said first stage of the condition in a higher amount than in said second stage, and the other gene is a expressed in said first stage of the condition in a lower amount than in said second stage of the condition,

correlating the expression level of the at least two genes to a standard level of expression determining the stage of the condition.

Thereby, it is possible to determine the biological condition in more details, such as determination of a stage and/or a grade of a tumor.

The methods above may be used for determining single gene expressions, however the invention also relates to a method of determining an expression pattern of a colon cell sample, comprising:

collecting sample comprising colon and/or rectum cells and/or expression products from colon and/or rectum cells,

determining the expression level of two or more genes in the sample, wherein at least one gene belongs to a first group of genes, said gene from the first gene group being expressed in a higher amount in normal tissue than in biological condition cells, and wherein at least one other gene belongs to a second group of genes, said gene from the second gene group being expressed in a lower amount in normal tissue than in biological condition cells, and the difference between the expression level of the first gene group in normal cells and biological condition cells being at least two-fold, obtaining an expression pattern of the colon and/or rectum cell sample.

Gene expression patterns may rely on one or a few genes, but more preferred gene expression patterns relies on expression from multiple genes, whereby a combined information from several genes is obtained.

Further, the invention relates to a method of determining an expression pattern of a colon cell sample independent of the proportion of submucosal, muscle, or connective tissue cells present, comprising:

determining the expression of one or more genes in a sample comprising cells, wherein the one or more genes exclude genes which are expressed in the submucosal, muscle, or connective tissue, whereby a pattern of expression is formed for the sample which is independent of the proportion of submucosal, muscle, or connective tissue cells in the sample.

The expression pattern may be used in a method according to this information, and accordingly, the invention also relates to a method of determining the presence or absence of a biological condition in human colon and/or rectum tissue comprising,

collecting a sample comprising cells from the tissue,

determining an expression pattern of the cells as defined above,

correlating the determined expression pattern to a standard pattern,

determining the presence or absence of the biological condition is said tissue.

as well as a method for determining the stage of a biological condition in animal tissue, comprising

collecting a sample comprising cells from the tissue,

determining an expression pattern of the cells as defined above,

correlating the determined expression pattern to a standard pattern,

determining the stage of the biological condition is said tissue.

The invention further relates to a method for reducing cell tumorigenicity of a cell, said method comprising

contacting a tumor cell with at least one peptide expressed by at least one gene selected from genes being expressed in an amount two-fold higher in normal cells than the amount expressed in said tumor cell, or

comprising

obtaining at least one gene selected from genes being expressed in an amount twofold higher in normal cells than the amount expressed in said tumor cell,

introducing said at least one gene into the tumor cell in a manner allowing expression of said gene(s), or

obtaining at least one nucleotide probe capable of hybridising with at least one gene of a tumor cell, said at least one gene being selected from genes being expressed in an amount one-fold lower in normal cells than the amount expressed in said tumor cell, and

introducing said at least one nucleotide probe into the tumor cell in a manner allowing the probe to hybridise to the at least one gene, thereby inhibiting expression of said at least one gene.

In a further aspect the invention relates to a method for producing antibodies against an expression product of a cell from a biological tissue, said method comprising the steps of

obtaining expression product(s) from at least one gene said gene being expressed as defined above,

immunising a mammal with said expression product(s) obtaining antibodies against the expression product.

The antibodies produced may be used for producing a pharmaceutical composition. Further, the invention relates to a vaccine capable of eliciting an immune response against at least one expression product from at least one gene said gene being expressed as defined above.

The invention furthermore relates to the use of any of the methods discussed above for producing an assay for diagnosing a biological condition in animal tissue.

Also, the invention relates to the use of a peptide as defined above as an expression product and/or the use of a gene as defined above and/or th use of a probe as defined above for preparation of a pharmaceutical composition for the treatment of a biological condition in animal tissue.

In a yet further aspect the invention relates to an assay for determining the presence or absence of a biological condition in animal tissue, comprising

at least one first marker capable of detecting a first expression level of at least one gene from a first gene group, wherein the gene from the first gene group is selected from genes expressed in normal tissue cells in an amount higher than expression in biological condition cells,

at least one second marker capable of detecting a second expression level of at least one gene from a second gene group, wherein the second gene group is selected from genes expressed in normal tissue cells in an amount lower than expression in biological condition cells.

In another aspect the invention relates to an assay for determining an expression pattern of a colon and/or rectum cell, comprising at least a first marker and a second marker, wherein the first marker is capable of detecting a gene from a first gene group as defined above, and the second marker is capable of detecting a gene from a second gene group as defined above.

### DETAILED DESCRIPTION OF THE INVENTION

Samples

The samples according to the present invention may be any tissue sample, it is however often preferred to conduct the methods according to the invention on epithelial tissue, such as epithelial tissue from the gastro-intestinal tract, in particular form colon and/or rectum. In particular the epithelial tissue may be mucosa.

The sample may be obtained by any suitable manner known to the man skilled in the art, such as a biopsy of the tissue, or a superficial sample scraped from the tissue. The sample may be prepared by forming a cell suspension made from the tissue, or by obtaining an extract from the tissue.

In one embodiment it is preferred that the sample comprises substantially only cells from said tissue, such as substantially only cells from mucosa of the colon-rectum.

Biological Condition

The methods according to the invention may be used for determining any biological condition, wherein said condition leads to a change in the expression of at least one gene, and preferably a change in a variety of genes.

Thus, the biological condition may be any malignant or premalignant condition, in particular in colon/rectum, such as an adenocarcinoma, a carcinoma, a teratoma, a sarcoma, and/or a lymphoma.

In relation to the gastrointestinal tract, the biological condition may also be colitis ulcerosa, Mb. Crohn, diverticulitis, adenomas.

Single Gene Expression Contra Expression Pattern

The expression level may be determined as single gene approaches, i.e. wherein the determination of expression from one or two or a few genes is conducted. It is preferred that expression from at least one gene from a first (normal) group is determined, said first gene group representing genes being expressed at a higher level in normal tissue, i.e. so-called suppressors, in combination with determination of expression of at least one gene from a second group, said second group representing genes being expressed at a higher level in tissue from the biological condition than in normal tissue, ie. so-called oncogenes. However, determination of the expression of a single gene whether belonging to the first group or second group is within the scope of the present invention. In this case it is preferred that the single gene is selected among genes having a very high change in expression level from normal cells to biological condition cells.

Another approach is determination of an expression pattern from a variety of genes, wherein the determination of the biological condition in the tissue relies on information from a variety of gene expression, i.e. rather on the combination of expressed genes than on the information from single genes.

Colorectal Tumors

The following data presented herein relates to colorectal tumors, and therefore the description has focused on the gene expression level as one way of identifying genes that lose function in cancer tissue. Genes showing a remarkable downregulation (or complete loss) of the expression level—measured as the mRNA transcript, during the malignant progression in colon from normal mucosa through Dukes A superficial tumors to Dukes B, slightly invasive tumors, to Dukes C that have spread to lymphnodes and finally to Dukes D that have metastasized to other organs, has been examined, as well as genes gaining importance during the differentiation towards malignancy.

Gene Groups

The present invention relates to a variety of genes identified either by an EST identification number and/or by a gene identification number. Both type of identification numbers relates to identification numbers of UniGene database, NCBI, build 18.

The various genes have been identified using Affymetrix arrays of the following product numbers:

Human Gene FL array 900 183

HU35K SubA 900 184

HU35K SubB 900 185

HU35K SubC 900 186

HU35K SubD 900 187

First Gene Group

Th first gene group relates to genes being expressed in normal tissue cells in an amount higher than expression in biological condition cells. The term “normal tissue cells” relates to c lls from the same type of tissue that is examined with respect to the biological condition in question. Thus, with respect to colorectal tumors, the normal tissue relates to colorectal tissue, in particular to colorectal mucosa.

The first gene group therefore relates to genes being downregulated in tumors, such genes being expected to serve as tumor suppressor genes, and they are of importance as predictive markers for the disease as loss of one or more of these may signal a poor outcome or an aggressive disease course. Furthermore, they may be important targets for therapy as restoring their expression level, e.g. by gene therapy, may suppress the malignant growth.

For a colorectal tissue sample a gene from the first gene group is preferably selected individually from genes comprising a sequence as identified below by EST

| UniGene number | Homologous to |
|---|---|
| RC_H04768_at | chrom 15 no homology |
| RC_Z39652_at | Y14593 APM-1 gene adipocyte-specific se- |
| | cretory protein; chrom 1q21.3-q23 |
| RC_H30270_at | chrom 18 PAAAA in colon & bladder no |
| | homology |
| RC_T47089_s_at | tenascin-X; tenascin-X precursor; unidenti- |
| | fied protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homolo- |
| | gy to mouse Pcbp1 - poly(rC)-binding |
| | protein 1 |
| RC_AA099820_at | BAC clone AC016778 |
| AA319615_at | secretory carrier membrane protein; secre- |
| | tory carrier membrane protein 2; chrom 15 |
| H07011_at | tetraspan NET-6 mRNA; transmembrane |
| | 4 superfamily; chrom 7 |
| RC_T68873_f_at | |
| RC_T40995_f_at | |
| RC_H81070_f_at | |
| RC_N30796_at | |
| RC_W37778_f_at | |
| RC_R70212_s_at | |
| RC_AA426330_at | |
| RC_N33927_s_at | |
| RC_T90190_s_at | |
| RC_AA447145_at | |
| RC_H75860_at | |
| RC_T71132_s_at | |

and from genes comprising a sequence as identified below

| | |
|---|---|
| “Human chromogranin A” “mRNA,” “complete cds” | J03915 |
| Human adipsin/complement factor D “mRNA,” comple- | M84526 |
| te cds | |
| Homo sapiens MLC-1V/Sb isoform gene | M24248 |
| Human aminopeptidase N/CD13 mRNA encoding | M22324 |
| aminopeptidase “N,” complete cds | |
| H. sapiens MT-1I mRNA | X76717 |
| H. sapiens GCAP-II gene | Z70295 |
| Human somatostatin I gene and flanks | J00306 |
| Human YMP “mRNA,” complete cds | U52101 |
| H. sapiens mRNA for beta subunit of epithelial amiloride- | X87159 |
| sensitive sodium channel | |
| Human K12 protein precursor “mRNA,” complete cds | U77643 |
| Human sulfate transporter (DTD) “mRNA,” complete cds | U14528 |
| Human transcription factor hGATA-6 “mRNA,” complete | U66075 |
| cds. | |
| H. sapiens SCAD “gene,” exon 1 and joining features | Z80345 |
| Human S-lac lectin L-14-II (LGALS2) gene | M87860 |
| Human mRNA for protein tyrosine phosphatase | D15049 |
| H. sapiens mRNA for tetranectin | X64559 |
| Human 11 kd protein “mRNA,” complete cds | U28249 |
| Human anti-mullerian hormone type II receptor precursor | U29700 |
| “gene,” complete cds | |
| Human heparin binding protein (HBp17) “mRNA,” complete | M60047 |
| cds | |
| Human ADP-ribosylation factor (hARF6) “mRNA,” complete | M57763 |
| cds | |
| beta-ADD = adducin beta subunit 63 kda isoform/membrane | S81083 |
| skeleton protein, beta-ADD = adducin beta subunit 63 kda | |
| isoform/membrane skeleton protein {alternatively spliced, | |
| exon 10 to 13 region} [human, Genomic, 1851 nt, segment | |
| 3 of 3]. | |
| Zinc Finger Protein Znf155 | HG4243- |
| | HT4513 |
| Human glucagon “mRNA,” complete cds | J04040 |
| H. sapiens mRNA for hair “keratin,” hHb5 | X99140 |
| Human tubulin-folding cofactor E “mRNA,” complete cds | U61232 |
| Human integrin alpha-3 chain “mRNA,” complete cds | M59911 |
| Human NACP gene | U46901 |
| H. sapiens mRNA for flavin-containing monooxygenase 5 | Z47553 |
| (FMO5) | |
| Human mRNA for ATF-a transcription factor | X52943 |
| H. sapiens intestinal VIP receptor related protein mRNA | X77777 |

and and from genes comprising a sequence as id ntified below

| | |
|---|---|
| Homo sapiens chromosome 16 BAC clone CIT987SK- | AF001548 |
| 815A9 complete sequence. | |
| Human mRNA for ATP synthase alpha “subunit,” complete | D14710 |
| cds | |
| Human mRNA for IgG Fc binding “protein,” complete | D84239 |
| cds | |
| H. sapiens mRNA for carcinoembryonic “antigen,” | X98311 |
| CGM2 | |
| “Homo sapiens (clone lamda-hPEC-3) phosphoenolpy- | L05144 |
| ruvate carboxykinase (PCK1)” “mRNA,” “complete | |
| cds” | |
| Human 11-beta-hydroxysteroid dehydrogenase type 2 | U26726 |
| “mRNA,” complete cds | |
| “Human intestinal mucin (MUC2)” “mRNA,” “complete cds” | L21998 |
| Human mRNA for KIAA0106 “gene,” complete cds | D14662 |
| metallothionein | V00594 |
| Human mRNA for IgG Fc binding “protein,” complete | D84239 |
| cds | |
| H. sapiens mRNA for carcinoembryonic “antigen,” | X98311 |
| CGM2 | |
| “Homo sapiens (clone lamda-hPEC-3) phosphoenolpy- | L05144 |
| ruvate carboxykinase (PCK1)” “mRNA,” “complete | |
| cds” | |
| metallothionein | V00594 |

In a preferred embodiment a gene from the first gene group is preferably selected individually from genes comprising a sequence as identified below by EST

| UniGene number | Homologous to |
|---|---|
| RC_H04768_at | chrom 15 no homology |
| RC_Z39652_at | Y14593 APM-1 gene adipocyte-specific se- |
| | cretory protein; chrom 1q21.3-q23 |
| RC_H30270_at | chrom 18 PAAAA in colon & bladder no |
| | homology |
| RC_AA279803_at | chrom 2 no homology |
| RC_R01646_at | chrom 13q32.1-33.3; AL159152; homolo- |
| | gy to mouse Pcbp1 - poly(rC)-binding |
| | protein 1 |
| RC_AA099820_at | BAC clone AC016778 |

and from genes comprising a sequence as identified below

| | |
|---|---|
| “Human chromogranin A” “mRNA,” “complete cds” | J03915 |
| Human adipsin/complement factor D “mRNA,” com- | M84526 |
| plete cds | |
| Homo sapi ns MLC-1V/Sb isoform gene | M24248 |
| Human aminopeptidase N/CD13 mRNA ncoding | M22324 |
| aminopeptidase “N,” complete cds | |
| H. sapiens MT-1I mRNA | X76717 |
| H. sapiens GCAP-II gene | Z70295 |
| Human somatostatin I gene and flanks | J00306 |

or selected individually from genes comprising a sequence as identified below by EST

| UniGene number | Homologous to |
|---|---|
| RC_H04768_at | chrom 15 no homology |
| RC_Z39652_at | Y14593 APM-1 gene adipocyte-specific se- |
| | cretory protein; chrom 1q21.3-q23 |
| RC_H30270_at | chrom 18 PAAAA in colon & bladder no |
| | homology |
| RC_T47089_s_at | tenascin-X; tenascin-X precursor; unidenti- |
| | fied protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homolo- |
| | gy to mouse Pcbp1 - poly(rC)-binding |
| | protein 1 |
| RC_AA099820_at | BAC clone AC016778 |
| AA319615_at | secretory carrier membrane protein; secre- |
| | tory carrier membrane protein 2; chrom 15 |
| H07011_at | tetraspan NET-6 mRNA; transmembrane |
| | 4 superfamily; chrom 7 |

In a more preferred embodiment a gene from the first gene group is selected individually from genes comprising a sequence as identified below by EST

| UniGene number | Homologous to |
|---|---|
| RC_H04768_at | chrom 15 no homology |
| RC_Z39652_at | Y14593 APM-1 gene adipocyte-specific se- |
| | cretory protein; chrom 1q21.3-q23 |
| RC_H30270_at | chrom 18 PAAAA in colon & bladder no |
| | homology |
| RC_T47089_s_at | tenascin-X; tenascin-X precursor; unidenti- |
| | fied protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homolo- |
| | gy to mouse Pcbp1 - poly(rC)-binding |
| | protein 1 |
| AA319615_at | secretory carrier membrane protein; secre- |
| | tory carrier membrane protein 2; chrom 15 |

In a most preferred embodiment a gene from the first gene group is selected individually from genes comprising a sequence as identified below by EST

| UniGene number | Homologous to |
|---|---|
| RC_T47089_s_at | tenascin-X; tenascin-X precursor; unidenti- |
| | fied protein |
| RC_W31906_at | secretagogin; dJ501N12.8 (putative protein) |
| | chrom 6 |
| RC_AA279803_at | chrom 2 no homology |
| AA319615_at | secretory carrier membrane protein; secre- |
| | tory carrier membrane protein 2; chrom 15 |

Second Gene Group

We have determined genes that are up-regulated (or gained de novo) during the malignant progression of colorectal cancer from normal tissue through Dukes A,B,C and to Dukes D. These genes are potential oncogenes and may be those genes that create or enhance the malignant growth of the cells. The expression level of these genes may serve as predictive markers for the disease course, as a high level may signal an aggressive disease course, and they may serve as targets for therapy, as blocking these genes by e.g. anti-sense therapy, or by biochemical means could inhibit, or slow, the tumor growth. Such up-regulated (or gained de novo) genes, oncogenes, may be classified according to the present invention as genes belonging to second genes group.

With respect to colorectal tumors genes belonging to the second gene group are preferably selected individually from genes comprising a sequence as identified below by EST

| UniGene number | Homologous to |
|---|---|
| RC_AA609013_s_at | microsomal dipeptidase (also on 6.8 k); |
| | chrom 16 |
| RC_AA232508_at | CGI-89 protein; unnamed protein product; |
| | hypothetical protein |
| RC_AA428964_at | serine protease-like protease; serine pro- |
| | tease homolog = NES1; normal epithelial cell- |
| | specific 1 |
| RC_T52813_s_at | dJ28O10.2 (G0S2 (PUTATIVE LYMPHO- |
| | CYTE G0/G1 SWITCH PROTEIN 2; chrom |
| | 1 |
| RC_AA075642_at | gp-340 variant protein; DMBT1/8kb.2 protein |
| RC_AA007218_at | chrom 13 no homology |
| RC_N33920_at | ubiquitin-like protein FAT10; diubiquitin; |
| | dJ271M21.6 (Diubiquitin); chrom 6 |
| RC_N71781_at | KIAA1199 protein, chrom 15 |
| RC_R67275_s_at | alpha-1 (type XI) collagen precursor; colla- |
| | gen, type XI, alpha 1; collagen type XI al- |
| | pha-1 isoform A; chrom 1 |
| RC_W80763_at | hypothetical protein; chrom 17 |
| RC_AA443793_at | chrom 7p22 AC006028 BAC clone |
| RC_AA034499_s_at | ZNF198 protein; zinc finger protein; FIM |
| | protein; Cys-rich protein; zinc finger protein |
| | 198; chrom 13 |
| RC_AA035482_at | chrom 5; AK022505 clone; CalcineurinB |
| | (weakly similar) |
| RC_AA024482_at | hypothetical protein; unnamed protein pro- |
| | duct; chrom 17 |
| RC_H93021_at | chrom 2; XM_004890 peptidylprolyl isomer- |
| | ase A (cyclophilin A) |
| RC_AA427737_at | no homology |
| RC_AA417078_at | chrom 7q31; AF017104 clone |
| M29873_s_at | cytochrome P450-IIB (hIIB3); 19q13.1- |
| | q13.2 |
| RC_H27498_f_at | |
| RC_T92363_s_at | |
| RC_N89910_at | |
| RC_W60516_at | |
| RC_AA219699_at | |
| RC_AA449450_at | |

and from genes comprising a sequence as identified below

| | |
|---|---|
| Homo sapiens (clones “MDP4,” MDP7) microsomal | J05257 |
| dipeptidase (MDP) “mRNA,” complete cds | |
| “Homo sapiens reg gene” “homologue,” “complete | L08010 |
| cds” | |
| H. sapiens mRNA for prepro-alpha2(I) collagen | Z74616 |
| “Human S-adenosylhomocysteine hydrolase (AHCY)” | M61832 |
| “mRNA,” “complete cds” | |
| Transcription Factor Iiia | HG4312- |
| | HT4582 |
| Human gene for melanoma growth stimulatory activity | X54489 |
| (MGSA) | |
| Human strom lysin-3 mRNA | X57766 |
| CDC25Hu2 = cdc25 + homolog “[human,” “mRNA,” 3118 | S78187 |
| nt] | |
| Human mRNA for cripto protein | X14253 |
| Human transformation-sensitive protein (IEF SSP 3521) | M86752 |
| “mRNA,” complet cds | |
| Human complement component 2 (C2) gene allele b | L09708 |
| H. sapiens mRNA for ITBA2 protein | X92896 |
| H. sapiens encoding CLA-1 mRNA | Z22555 |
| “Human fibroblast growth factor receptor 4 (FGFR4)” | L03840 |
| “mRNA,” “complete cds“ | |
| “”“Fibronectin,” “Alt. Splice 1” | HG3044- |
| | HT3742 |
| tyk2 | X54667 |
| Human mRNA for B-myb gene | X13293 |
| “Human phosphofructokinase (PFKM)” “mRNA,” “complete | U24183 |
| cds” | |
| Human pre-B cell enhancing factor (PBEF) “mRNA,” com- | U02020 |
| plete cds | |
| Human SH2-containing inositol 5-phosphatase (hSHIP) | U57650 |
| “mRNA,” complete cds | |
| Human interleukin 8 (IL8) “gene,” complete cds | M28130 |
| “Human lamin B receptor (LBR)” “mRNA,” “complete cds” | L25931 |
| H. sapiens mRNA for protein tyrosine phosphatase | Z48541 |
| Human mRNA for unc-18 “homologue,” complete cds | D63851 |
| H. sapiens mRNA for Zn-alpha2-glycoprotein | X59766 |
| | Z25521 |
| “Human asparagine synthetase” “mRNA,” “complete cds” | M27396 |
| Human hepatitis delta antigen interacting protein A (dipA) | U63825 |
| “mRNA,” complete cds | |
| Human splicesomal protein (SAP 61) “mRNA,” complete | U08815 |
| cds | |
| Human protein kinase C-binding protein RACK7 “mRNA,” | U48251 |
| partial cds | |
| Human MAC30 “mRNA,” 3′ end | L19183 |
| Human thrombospondin 2 (THBS2) “mRNA,” complete cds | L12350 |
| “Human nicotinamide N-methyltransferase (NNMT)” | U08021 |
| “mRNA,” “complete cds” | |
| H. sapiens mRNA for type I interstitial collagenase | X54925 |
| Human cytochrome b561 gene | U29463 |
| Human H19 RNA “gene,” complete cds (spliced in sili- | M32053 |
| co) | |
| Human collagen type XVIII alpha 1 (COL18A1) “mRNA,” | L22548 |
| partial cds | |
| Human clone 23733 “mRNA,” complete cds. | U79274 |
| Human transforming growth factor-beta induced gene pro- | M77349 |
| duct (BIGH3) “mRNA,” complete cds | |
| “Human breast epithelial antigen BA46” “mRNA,” “com- | U58516 |
| plete cds” | |
| | X57351 |
| H. sapiens NGAL gene | X99133 |
| Human mRNA for MDNCF (monocyte-derived neutro- | Y00787 |
| phil chemotactic factor) | |
| H. sapiens EF-1 delta gene encoding human elongation | Z21507 |
| factor-1-delta | |
| H. sapiens mRNA for prepro-alpha1(I) collagen | Z74615 |
| Nuclear Factor Nf-II6 | HG3494- |
| | HT3688 |
| | U29175 |
| “HNL = neutrophil lipocalin” “[human,” “ovarian | S75256 |
| cancer cell line” “OC6,” “mRNA” “Partial,” “534 | |
| nt]. /qb = S75256 /ntype = RNA” | |

In a preferred embodiment the genes belonging to the second gene group are preferably selected individually from genes comprising a sequence as identified below by EST

| UniGene number | Homologous to |
|---|---|
| RC_AA007218_at | chrom 13 no homology |
| RC_AA443793_at | chrom 7p22 AC006028 BAC clone |
| RC_AA035482_at | chrom 5; AK022505 clone; CalcineurinB |
| | (weakly similar) |
| RC_H93021_at | chrom 2; XM_004890 peptidylprolyl isomer- |
| | ase A (cyclophilin A) |
| RC_AA427737_at | no homology |
| RC_AA417078_at | chrom 7q31; AF017104 clone |

and from genes comprising a sequence as identified below

In another preferred embodiment genes from the second gene group are selected individually from genes comprising a sequence as identified below

| UniGene number | Homologous to |
|---|---|
| RC_AA609013_s_at | microsomal dipeptidase (also on 6.8 k); |
| | chrom 16 |
| RC_AA232508_at | CGI-89 protein; unnamed protein product; |
| | hypothetical protein |
| RC_AA428964_at | serine protease-like protease; serine pro- |
| | tease homolog = NES1; normal epithelial cell- |
| | specific 1 |
| RC_T52813_s_at | dJ28O10.2 (G0S2 (PUTATIVE LYMPHO- |
| | CYTE G0/G1 SWITCH PROTEIN 2; chrom |
| | 1 |
| RC_AA075642_at | gp-340 variant protein; DMBT1/8kb.2 protein |
| RC_AA007218_at | chrom 13 no homology |
| RC_N33920_at | ubiquitin-like protein FAT10; diubiquitin; |
| | dJ271M21.6 (Diubiquitin); chrom 6 |
| RC_N71781_at | KIAA1199 protein, chrom 15 |
| RC_R67275_s_at | alpha-1 (type XI) collagen precursor; colla- |
| | gen, type XI, alpha 1; collagen type XI al- |
| | pha-1 isoform A; chrom 1 |
| RC_W80763_at | hypothetical protein; chrom 17 |
| RC_AA443793_at | chrom 7p22 AC006028 BAC clone |
| RC_AA034499_s_at | ZNF198 protein; zinc finger protein; FIM |
| | protein; Cys-rich protein; zinc finger protein |
| | 198; chrom 13 |
| RC_AA035482_at | chrom 5; AK022505 clone; CalcineurinB |
| | (weakly similar) |
| RC_AA024482_at | hypothetical protein; unnamed protein pro- |
| | duct; chrom 17 |
| RC_H93021_at | chrom 2; XM_004890 peptidylprolyl isomer- |
| | ase A (cyclophilin A) |
| RC_AA427737_at | no homology |
| RC_AA417078_at | chrom 7q31; AF017104 clone |
| M29873_s_at | Cytochrome P450-IIB (hIIB3); 19q13.1- |
| | q13.2 |

In a more preferred embodiment genes from the second gene group are selected individually from genes comprising a sequence as identified below

| UniGene number | Homologous to |
|---|---|
| RC_AA609013_s_at | microsomal dipeptidase (also on 6.8 k); |
| | chrom 16 |
| RC_AA232508_at | CGI-89 protein; unnamed protein product; |
| | hypothetical protein |
| RC_AA428964_at | serine protease-like protease; serine pro- |
| | tease homolog = NES1; normal epithelial cell- |
| | specific 1 |
| RC_AA075642_at | gp-340 variant protein; DMBT1/8kb.2 protein |
| RC_AA007218_at | chrom 13 no homology |
| RC_N33920_at | ubiquitin-like protein FAT10; diubiquitin; |
| | dJ271M21.6 (Diubiquitin); chrom 6 |
| RC_N71781_at | KIAA1199 protein, chrom 15 |
| RC_R67275_s_at | alpha-1 (type XI) collagen precursor; colla- |
| | gen, type XI, alpha 1; collagen type XI al- |
| | pha-1 isoform A; chrom 1 |
| RC_W80763_at | hypothetical protein; chrom 17 |
| RC_AA034499_s_at | ZNF198 protein; zinc finger protein; FIM |
| | protein; Cys-rich protein; zinc finger protein |
| | 198; chrom 13 |
| RC_AA035482_at | chrom 5; AK022505 clone; CalcineurinB |
| | (weakly similar) |
| RC_AA024482_at | hypothetical protein; unnamed protein pro- |
| | duct; chrom 17 |
| RC_H93021_at | chrom 2; XM_004890 peptidylprolyl isomer- |
| | ase A (cyclophilin A) |
| RC_AA427737_at | no homology |
| RC_AA417078_at | chrom 7q31; AF017104 clone |
| M29873_s_at | cytochrome P450-IIB (hIIB3); 19q13.1- |
| | q13.2 |

In an even more preferred embodiment genes from the second gene group are selected individually from genes comprising a sequence as identified below

| UniGene number | Homologous to |
|---|---|
| RC_AA609013_s_at | microsomal dipeptidase (also on 6.8 k); |
| | chrom 16 |
| RC_AA007218_at | chrom 13 no homology |
| RC_AA035482_at | chrom 5; AK022505 clone; CalcineurinB |
| | (weakly similar) |
| RC_H93021_at | chrom 2; XM_004890 peptidylprolyl isomer- |
| | ase A (cyclophilin A) |
| RC_AA427737_at | no homology |
| RC_AA417078_at | chrom 7q31; AF017104 clone |

such as a sequence as identified below

| UniGene number | Homologous to |
|---|---|
| RC_W80763_at | hypothetical protein; chrom 17 |

The genes from the second gene group discussed above are preferably genes being expressed in all stages of the biological condition, such as all Dukes stages of a colorectal tumor, to be used for determining the biological condition.

Number of Genes

As discussed above, it is possible to use a single gene approach determining the expression of one of the genes only, in order to determine th biological condition of the tissue. This is particularly relevant for genes mentioned in the tables in Experiments, since these genes have been determined as having a strong indicativity per gene. It is however preferred that expression from at least one gene from the first group as well as expression from one gene from the second group is determined to obtain a more statistically significant result, that is more independent of the expression level of the individual gene. In a preferred embodiment expression from more genes from both groups are determined, such as determination of expression from at least two genes from either of the gene groups, such as determination of expression from at least three genes from either of the gene groups, such as determination of expression from at least four genes from either of the gene groups, such as determination of expression from at least five genes from either of the gene groups, such as determination of expression from at least six genes from either of the gene groups, such as determination of expression from at least seven genes from either of the gene groups.

A pattern of characteristic expression of one gene can be useful in characterizing a cell type source or a stage of disease. However, more genes may be usefully analyzed. Useful patterns include expression of at least one, two, three, five, ten, fifteen, twenty, twenty-five, fifty, seventy-five, one hundred or several hundred informative genes.

Expression Level

Using the results provided in the accompanying figures and tables, a gene is indicated as being expressed if an intensity value of greater than or equal to 20 is shown. Conversely, an intensity value of less than 20 indicates that the gene is not expressed above background levels. Comparison of an expression pattern to another may score a change from expressed to non-expressed, or the reverse. Alternatively, changes in intensity of expression may be scored, either increases or decreases. Any statistically significant change can be used. Typically changes which are greater than 2-fold are suitable. Changes which are greater than 5-fold are highly significant.

The present invention in particular relates to methods using genes wherein the ratio of the expression level in normal tissue to biological condition tissue for suppressor genes or vice versa of the expression level in biological condition tissue to normal tissue for condition genes is as high as possible, such as at least two-fold change in expression, such as at least three-fold, such as at least four fold, such as at least five fold, such as at least six fold, such as at least ten fold, such as at least fifteen fold, such as at least twenty fold.

Stages and Grades

Stage of a colorectal tumor indicates how deep the tumor has penetrated. Superficial tumors are termed Dukes A and Dukes B and Dukes C are used to describe increasing degrees of penetration into the muscle. The grade of a colorectal tumor is expressed on a scale of I-IV (1-4). The grade reflects the cytological appearance of the cells. Grade I cells are almost normal. Grade II cells are slightly deviant. Grade III cells are clearly abnormal. And Grade IV cells are highly abnormal.

It is important to classify the stage of a cancer disease, as superficial tumors may require a less intensive treatment than invasive tumors. We have therefore used the expression level of genes to identify genes whose expression can be used to identify a certain stage of the disease. We have divided these “Classifiers” into those which can be used to identify Dukes A, B, C, and D stages. We expect that measuring the transcript level of one or more of these genes will lead to a classifier that can add supplementary information to the information obtained from the pathological Dukes classification. For example we believe that gene expression levels that signify a Dukes C will be unfavourable to detect in a Dukes A tumor, as they may signal that the Dukes A tumor has the potential to become a Dukes C tumor. The opposite is probably also true, that an expression level that signify Dukes A will be favorable to have in a Dukes C tumor. In that way independent information may be obtained from Dukes pathological classification and a classification based on gene expression levels is made.

Thus, in one embodiment the invention relates to a method as described above further comprising the steps of determining the stage of a biological condition in the animal tissue, comprising assaying a third expression level of at least one gene from a third gene group, wherein a gene from said second gene group, in one stage, is expressed differently from a gene from said third gene group.

In another aspect the invention relates to method of determining the stage of a biological condition in animal tissue,

comprising collecting a sample comprising cells from the tissue,

assaying the expression of at least a first stage gene from a first stage gene group and/or at least a second stage gene from a second stage gene group, wherein at least one of said genes is expressed in said first stage of the condition in a higher amount than in said second stage, and the other gene is a expressed in said first stage of the condition in a lower amount than in said second stage of the condition,

correlating the expression level of the assessed genes to a standard level of expression determining the stage of the condition.

The method of determining the stage of a tumor may be combined with determination of the biological condition or may be an independent method as such. The difference in expression level of a gene from one stage to the expression level of the gene in another group is preferably at least two-fold, such as at least three-fold.

Thus, the invention relates to a method of determining the stage of a colorectal tumor, wherein the stage is selected from colon cancer stages Dukes A, Dukes B, Dukes C, and Dukes D, comprising assaying at least the expression of Dukes A stage gene from a Dukes A stage gene group, at least one Dukes B stage gene from a Dukes B stage gene group, at least the expression of Dukes C stage gene from a Dukes C stage gene group, and/or at least one Dukes D stage gene from a Dukes D stage gene group, wherein at least one gene from each gene group is expressed in a significantly different amount in that stage than in one of the other stages.

The genes selected may be a gene from each gene group being expressed in a significantly higher amount in that stage than in one of the other stages, such as:

a Dukes A stage gene selected individually from any gene comprising a sequence as identified below as EST

| | |
|---|---|
| RC_AA599199_at | ALU seq. |
| RC_R12694_at | unnamed protein product |
| | BAA91641, chrom 10 |
| RC_H91325_s_at | aldolase B; aldolase B (aa 1- |
| | 364); chrom 9 |
| RC_N51709_at | chrom X |
| RC_N72610_at | — |
| RC_N69263_at | chrom 10; AK026414 clone |
| | (only 108 nt hom) |
| RC_T15817_f_at | iNOS, inducible nitric oxide |
| | synthase |

| | |
|---|---|
| RC_F03077_f | chromosome 17, clone |
| | hRPC.15 |
| RC_AA599199 | Alu seq |
| RC_AA207015 | clone RP4-733M16 on chromo- |
| | some 1p36.11-36.23 |
| RC_AA234916 | chromosome 19 clone CTC- |
| | 461 H2 |
| RC_N92239_a | Wnt inhibitory factor-1 (WIF-1), |
| | chromosome 12 |
| RC_N93958_s | phospholipase A2, group X |
| | (PLA2G10), |
| U95301_at | phospholipase A2, group X |
| | (PLA2G10), |
| RC_AA426330 | chromosome 17, clone |
| | hRPC.1110_E_20 |
| RC_AA024658 | clone SCb-254N2 |
| | (UWGC:rg254N02) from 6p21 |
| RC_H88540_a | heat shock protein 90, 1q21.2- |
| | q22 |

or any gene comprising a sequence as identified below

| | |
|---|---|
| D87444_at | Human mRNA for KIAA0255 “gene,” complete cds |
| U18291_at | Human CDC16Hs “mRNA,” complete cds |
| L76568_xpt3_f_at | S26 from Homo sapiens excision and cross link repair protein |
| | (ERCC4) “gene,” complete genomic sequence. /gb = L76568 |
| | /ntype = DNA /annot = exon |
| U45328_s_at | “Human ubiquitin-conjugating enzyme (UBE2I)” “mRNA,” “complete |
| | cds” |
| Z14982_rna1_at | H. sapiens gene for major histocompatibility complex encoded protea- |
| | some subunit LMP7. |
| AD000092_cds7_s | RAD23A gene (human RAD23A homolog) extracted from Homo |
| _at | sapiens DNA from chromosome 19p13.2 cosmids “R31240,” R30272 |
| | and R28549 containing the “EKLF,” “GCDH,” “CRTC,” and RAD23A |
| | “genes,” genomic sequence |
| D86973_at | Human mRNA for KIAA0219 “gene,” partial cds |
| X81636_at | H. sapiens clathrin light chain a gene |
| M59916_at | Human acid sphingomyelinase (ASM) “mRNA,” complete cds |
| X85781_s_at | “H. sapiens NOS2” “gene,” “exon 27 /gb = X85781 /ntype = DNA |
| | /annot = exon” |
| M57731_s_at | “Human gro-beta” “mRNA,” “complete cds” |
| U49188_at | Human placenta (Diff33) “mRNA,” complete cds |
| X53800_s_at | Human mRNA for macrophage inflammatory protein-2beta (MIP2beta) |
| U56816_at | Human kinase Myt1 (Myt1) “mRNA,” complete cds. |
| HG1067- | Mucin (Gb:M22406) |
| HT1067_r_at | |

| | |
|---|---|
| Human migration inhibitory factor-related protein 8 (MRP8) | M21005 |
| “gene,” complete cds | |
| Human acyloxyacyl hydrolase “mRNA,” complete cds | M62840 |
| Human PEP19 (PCP4) “mRNA,” complete cds | U52969 |
| H. sapiens Humig mRNA | X72755 |
| H. sapiens PISSLRE mRNA | X78342 |
| H. sapiens mRNA for twist “protein,” partial. | Y11180 |
| /gb = Y11180 /ntype = RNA | |
| Human mRNA for TGF-beta superfamily “protein,” com- | AB000584 |
| plete cds | |
| Human mRNA for “MSS1,” complete cds | D11094 |
| Human complement factor B “mRNA,” complete cds | L15702 |
| “Homo sapiens GTP-binding protein | M28213 |
| (RAB2)” “mRNA,” “complete cds” | |
| Human translational initiation factor 2 beta subunit (eIF-2- | M29536 |
| beta) “mRNA,” complete cds | |
| Human E16 “mRNA,” complete cds | M80244 |
| IEX-1 = radiation-inducible immediate-early | S81914 |
| gene “[human,” “placenta,” | |
| mRNA “Partial,” 1223 nt] | |
| Human CDC16Hs “mRNA,” complete cds | U18291 |
| Human DD96 “mRNA,” complete cds | U21049 |
| Human (memc) “mRNA,” 3′UTR. /gb = U30999 | U30999 |
| /ntype = RNA | |
| “Human ubiquitin-conjugating enzyme | U45328 |
| (UBE2I)” “mRNA,” “complete cds” | |
| “Human fetal brain glycogen phosphorylase | U47025 |
| B” “mRNA,” “complete cds” | |
| “Human BTG2 (BTG2)” “mRNA,” “complete cds” | U72649 |
| Human jun-B mRNA for JUN-B protein | X51345 |
| Human chaperonin 10 “mRNA,” complete cds | U07550 |
| H. sapiens RING4 cDNA | X57522 |
| H. sapiens genes TAP1, TAP2, LMP2, LMP7 and DOB. | X66401 |
| H. sapiens mRNA for alpha 4 protein | Y08915 |
| Homo sapiens interleukin-1 receptor-associated kinase | L76191 |
| (IRAK) “mRNA,” complete cds | |
| “Human von Willebrand factor” “mRNA,” “3′ | M10321 |
| end” | |
| Human chromosome segregation gene homolog CAS | U33286 |
| “mRNA,” complete cds | |
| Human Bruton's tyrosine kinase-associated protein-135 | U77948 |
| “mRNA,” complete cds. | |
| “Human KH type splicing regulatory protein KSRP” | U94832 |
| “mRNA,” “complete cds.” | |
| H. sapiens ADE2H1 mRNA showing homologies to SAICAR | X53793 |
| synthetase and AIR carboxylase of the purine pathway (EC | |
| “6.3.2.6,” EC 4.1.1.21) | |

a Dukes B stage gene is selected individually from any gene comprising a sequence as identified below

| | | |
|---|---|---|
| RC_T67463_s_at | | cathepsin O2; X; K |
| RC_W94688_at | | perilipin |
| RC_AA126743_at | | Z97200 PAC chrom 1q24; |
| | | PMX1 homeobox gene |
| RC_AA236547_at | | no homology |
| RC_AA255567_at | | angiopoietin-related protein-2; |
| | | angiopoietin-like 2 |
| RC_AA421256_at | | — |
| RC_AA386386_s | PPPPP | — |
| _at | | |
| RC_AA452549_at | PPPPP | PRO1659; hypothetical protein |
| | | chrom 11 |

| | |
|---|---|
| M63262_at | 5-lipoxygenase activating protein (FLAP), |
| | 13q12 |
| R67290_at | Interleukine 14 |
| N36619_at | |
| L19161_at | translation initiation factor 2, subunit 3″, |
| | Xp22.2-22.1 |
| RC_AA496035 | Chromosome 1? (TIGR) |
| L29217_s_at | CDC-like kinase 3 (CLK3), 15q24 |
| RC_W73194_a | Dermatoponin, 1q12-q23 |
| RC_N69507_a | hypothetical protein PRO1847 (Alu accord- |
| | ing to TIGR) |
| RC_H15814_s | adipose most abundant gene transcript 1 |
| M84526_at | D component of complement (adipsin) |

or any gene comprising a sequence as identified below

| | |
|---|---|
| U57316_at | Human GCN5 (hGCNS) “gene,” complete cds |
| X66839_at | H. sapiens MaTu MN mRNA for p54/58N protein |
| J04599_at | Human hPGI mRNA encoding bone small proteoglycan I “(biglycan),” com- |
| | plete cds |
| X57579_s_at | H. sapiens activin beta-A subunit (exon 2) |
| J02874_at | Human adipocyte lipid-binding “protein,” complete cds |
| M11749_at | Human Thy-1 glycoprotein “gene,” complete cds |
| U06863_at | Human follistatin-related protein precursor “mRNA,” complete cds |
| U51010_s_at | “Human nicotinamide N-methyltransferase” “gene,” “exon 1 and 5′ flanking |
| | region. /gb = U51010 /ntype = DNA /annot = exon” |
| U08021_at | “Human nicotinamide N-methyltransferase (NNMT)” “mRNA,” “complete |
| | cds” |
| HG3044- | “”“Fibronectin,” “Alt. Splice 1” |
| HT3742_s_at | |
| X02761_s_at | Human mRNA for fibronectin (FN precursor) |
| X02544_at | Human mRNA for alpha1-acid glycoprotein (orosomucoid) |
| M62505_at | Human C5a anaphylatoxin receptor “mRNA,” complete cds |
| J05070_at | Human type IV collagenase “mRNA,” complete cds |
| U16306_at | Human chondroitin sulfate proteoglycan versican V0 splice-variant precursor |
| | peptide “mRNA,” complete cds |
| M14218_at | Human argininosuccinate lyase “mRNA,” complete cds |
| L77567_s_at | “Homo sapiens mitochondrial citrate transport protein (CTP)” “mRNA,” “3′ |
| | end” |
| M63391_rna1 | Human desmin gene, complete cds. |
| _at | |
| D13643_at | Human mRNA for KIAA0018 “gene,” complete cds |
| D79985_at | Human mRNA for KIAA0163 “gene,” complete cds |

| | |
|---|---|
| Human adipocyte lipid-binding “protein,” complete cds | J02874 |
| Human A1 protein “mRNA,” complete cds | U29680 |
| Human LGN protein “mRNA,” complete cds | U54999 |
| Human skeletal muscle LIM-protein SLIM2 “mRNA,” partial | U60116 |
| cds | |
| Human mRNA for alpha1-acid glycoprotein (orosomucoid) | X02544 |
| Human mRNA for fibronectin receptor alpha subunit | X06256 |
| H. sapiens P1-Cdc21 mRNA | X74794 |
| H. sapiens mRNA for fibulin-2 | X82494 |
| H. sapiens 5T4 gene for 5T4 Oncofetal antigen | Z29083 |
| Homo sapiens mRNA for osteoblast specific factor 2 (OSF- | D13666 |
| 2os) | |
| Mac25 | HG987- |
| | HT987 |
| “Human lysozyme” “mRNA,” “complete cds with | J03801 |
| an Alu repeat in the 3′ flank” | |
| Human metalloproteinase (HME) “mRNA,” complete cds | L23808 |
| Human alpha-1 collagen type IV gene, exon 52. | M26576 |
| Human lumican “mRNA,” complete cds | U21128 |
| Human mRNA for fibronectin (FN precursor) | X02761 |
| Human mRNA fragment for elongation factor TU (N- | X03689 |
| terminus). /gb = X03689 /ntype = RNA | |
| Human mRNA for type IV collagen alpha-2 chain | X05610 |
| Human mRNA for collagen VI alpha-1 C-terminal globular | X15880 |
| domain | |
| “H. sapiens,” gene for Membrane cofactor protein | X59405 |
| H. sapiens SOD-2 gene for manganese superoxide dismu- | X65965 |
| tase. /gb = X65965 /ntype = DNA /annot = exon | |
| H. sapiens NMB mRNA | X76534 |
| H. sapiens vimentin gene | Z19554 |
| Human chaperonin 10 “mRNA,” complete cds | U07550 |
| H. sapiens RING4 cDNA | X57522 |
| H. sapiens genes TAP1, TAP2, LMP2, LMP7 and DOB. | X66401 |
| H. sapiens mRNA for alpha 4 protein | Y08915 |
| Homo sapiens interleukin-1 receptor-associated kinase | L76191 |
| (IRAK) “mRNA,” complete cds | |
| “Human von Willebrand factor” “mRNA,” “3′ | M10321 |
| end” | |
| Human chromosome segregation gene homolog CAS | U33286 |
| “mRNA,” complete cds | |
| Human Bruton's tyrosine kinase-associated protein-135 | U77948 |
| “mRNA,” complete cds. | |
| “Human KH type splicing regulatory protein KSRP” | U94832 |
| “mRNA,” “complete cds.” | |
| H. sapiens ADE2H1 mRNA showing homologies to SAICAR | X53793 |
| synthetase and AIR carboxylase of the purine pathway (EC | |
| “6.3.2.6,” EC 4.1.1.21) | |
| “”“Globin,” “Beta” | HG1428- |
| | HT1428 |
| “Human alpha-1 collagen type I” “gene,” “3′ | M55998 |
| end” | |
| H. sapiens mRNA for SOX-4 protein | X70683 |
| “Human mRNA for collagen binding | D83174 |
| protein” “2,” “complete cds” | |
| Human SPARC/osteonectin “mRNA,” complete cds | J03040 |
| Human PRAD1 mRNA for cyclin | X59798 |

a Dukes C stage gene is selected individually from any gene comprising a sequence as identified below

| | | |
|---|---|---|
| RC_D45556_at | | chrom 15; AL390085 clone |
| RC_W86214_at | | |
| RC_AA039439_s | | novel gene KIAA0134 protein |
| _at | | 19q13.3 |
| RC_AA128935_at | | |
| RC_AA134158_s | | class I homeodomain; homeo- |
| _at | | box protein, chrom 7 |
| RC_AA232646_at | | chrom 17, AF266756 sphingo- |
| | | sine kinase (SPHK1 |
| RC_AA401184_at | | no homology |
| RC_AA436840_at | | |
| RC_AA488655_at | | |
| RC_AA181902_at | PPPPP | AC007201 on chrom 19 (only |
| | | 80 nt hom) |

| | |
|---|---|
| RC_AA122350 | chromosome 8 |
| AA374109_at | spondin 2, extracellular matrix |
| | protein, chromosome 4 |
| RC_AA621755 | transcription factor Dp-2, 3q23 |
| RC_AA442069 | sodium channel 2, 12q12 |
| RC_T40767_a | chromosome 19 |
| RC_AA488655 | Mus? |
| RC_AA398908 | |
| RC_AA447764 | hypothetical protein, chromosome 4 |
| RC_N69136_a | |

or any gene comprising a sequence as identified below

| | |
|---|---|
| M20681_at | Human glucose transporter-like protein-III “(GLUT3),” |
| | complete cds |
| D50914_at | Human mRNA for KIAA0124 “gene,” partial cds |
| L37362_at | Homo sapiens (clone d2-115) kappa opioid receptor |
| | (OPRK1) “mRNA,” complete cds |
| X66114_rna1 | H. sapiens gene for 2-oxoglutarate carrier protein. |
| _at | |
| M32053_at | Human H19 RNA “gene,” complete cds (spliced in |
| | silico) |
| Y00787_s_at | Human mRNA for MDNCF (monocyte-derived |
| | neutrophil chemotactic factor) |
| U64444_at | Human ubiquitin fusion-degradation protein (UFD1L) |
| | “mRNA,” complete cds |
| X95325_s_at | H. sapiens mRNA for DNA binding protein A variant |
| X02419_rna1 | H. sapiens uPA gene |
| _s_at | |
| X57522_at | H. sapiens RING4 cDNA |
| AB001325_at | Human AQP3 gene for aquaporine 3 (water “channel),” |
| | partail cds |
| AB002315_at | Human mRNA for KIAA0317 “gene,” complete cds. |
| | /gb = AB002315 /ntype = RNA |
| L12760_s_at | “Human phosphoenolpyruvate carboxykinase (PCK1)” |
| | “gene,” “complete cds with repeats” |
| M80899_at | Human novel protein AHNAK “mRNA,” partial |
| | sequence |

| | |
|---|---|
| Ribosomal Protein L39 Homolog | HG2874- |
| | HT3018 |
| Homo sapiens (clone d2-115) kappa opioid receptor | L37362 |
| (OPRK1) “mRNA,” complete cds | |
| Human kell blood group protein mRNA | M64934 |
| | U73167 |
| Human cancellous bone osteoblast mRNA for serin pro- | D87258 |
| tease with IGF-binding “motif,” complete cds | |
| Human interferon-inducible protein 27-Sep “mRNA,” com- | J04164 |
| plete cds | |
| “Human sickle cell beta-globin” “mRNA,” “complete cds” | M25079 |
| | M29277 |
| “Human spermidine synthase” “mRNA,” “complete cds” | M34338 |
| Human copine I “mRNA,” complete cds | U83246 |
| “”“Globin,” “Beta” | HG1428- |
| | HT1428 |
| “Human alpha-1 collagen type I” “gene,” “3′ end” | M55998 |
| H. sapiens mRNA for SOX-4 protein | X70683 |
| “Human mRNA for collagen binding protein” “2,” “complete | D83174 |
| cds” | |
| Human SPARC/osteonectin “mRNA,” complete cds | J03040 |
| Human PRAD1 mRNA for cyclin | X59798 |

a Dukes D stage gene is selected individually from any gene comprising a sequence as identified below

| | | |
|---|---|---|
| RC_N91920_at | AAAAP | chrom 16p12-p11.2; |
| | | XN_007994 retinoblastoma |
| | | binding protein |
| RC_AA621601_at | AAAAP | chrom 17 XM_009868 RAB36 |
| | | ARS oncogene family |

| | |
|---|---|
| RC_AA121433 | Axin, chromosome 16 |
| RC_N91920_a | RB protein binding protein, |
| | chromosome 16 |
| RC_AA621601 | GTP-binding protein Rab36, |
| | chromosome 17 |
| RC_AA454020 | NADPH quinone oxidoreduc- |
| | tase homolog; p53 induced, |
| | chromosome 2 |
| RC_Z39652_a | APM-1 gene, chromosome 18 |

or any gene comprising a sequence as identified below

| | |
|---|---|
| X17644_s_{—} | Human GST1-Hs mRNA for GTP-binding protein |
| at | |
| Y12812_at | H. sapiens RFXAP mRNA |
| X60486_at | H. sapiens H4/g gene for H4 histone |
| X52221_at | H. sapiens ERCC2 “gene,” exons 1 & 2 |
| | (partial) |
| L06175_at | Homo sapiens P5-1 “mRNA,” complete cds |
| Z48481_at | H. sapiens mRNA for membrane-type matrix |
| | metalloproteinase 1 |
| X54232_at | Human mRNA for heparan sulfate proteaglycan |
| | (glypican) |
| L08010_at | “Homo sapiens reg gene” “homologue,” |
| | “complete cds” |
| L27706_at | Human chaperonin protein (Tcp20) gene |
| | complete cds |
| L15533_rna1 | Homo sapiens pancreatits-associated protein |
| _at | (PAP) gene, complete cds. |
| X51408_at | Human mRNA for n-chimaerin |
| K02765_at | Human complement component C3 “mRNA,” |
| | alpha and beta “subunits,” complete cds |
| U38904_at | Human zinc finger protein C2H2-25 “mRNA,” |
| | complete cds |

| | |
|---|---|
| Homo sapiens FRG1 “mRNA,” complete cds | L76159 |
| Human cyclin protein “gene,” complete cds | M15796 |
| Human U2 small nuclear RNA-associated B″ antigen | M15841 |
| “mRNA,” complete cds | |
| Human mRNA export protein Rae1 (RAE1) “mRNA,” com- | U84720 |
| plete cds. | |
| Human protease-activated receptor 3 (PAR3) “mRNA,” | U92971 |
| complete cds. | |
| H. sapiens mRNA for mediator of receptor-induced toxicity | X84709 |
| H. sapiens RFXAP mRNA | Y12812 |
| Human mRNA for “Qip1,“ complete cds | AB002533 |
| Human mRNA for transferrin receptor | X01060 |
| “metastasis-associated gene ”“[human,”“ highly metastatic | S79219 |
| lung cell subline ”“Anip[937],”“ mRNA ”“Partial,”“ 978 nt]” | |

The genes selected may b a gene from each gene group being expressed in a significantly lower amount in that stage than in one of the other stages, such as: a Dukes A stage gene is selected individually from any gene comprising a sequence as identified below

| | | |
|---|---|---|
| RC_N32411_f_at | PAPPP | Myc-associated zinc-finger |
| | | protein of human islet; chrom |
| | | 16 |
| RC_AA243858_at | PAPPP | KIAA0882 protein |
| RC_AA486283_at | PAPPP | ras-like protein; ras-related C3 |
| | | botulinum toxin substrate; |
| | | dJ20J23 |
| RC_AA490930_at | PAPPP | chrom 18; KIAA1468 protein |
| RC_H54088_s_at | PPPPP | ribosomal protein L41 |
| RC_H59052_f_at | PPPPP | fungal sterol-C5-desaturase |
| | | homolog; ORF; thymosin beta-4 |
| RC_R49198_s_at | PPPPP | — |
| RC_T73572_f_at | PPPPP | ferritin L-chain; L apoferritin |
| RC_AA477483_at | PPPPP | no matching est |

or any gene comprising a sequence as identified below

| | |
|---|---|
| Homo sapiens SKB1Hs “mRNA,” complete cds. | AF015913 |
| /gb = AF015913 /ntype = RNA | |
| Mucin (Gb:M22406) | HG1067- |
| | HT1067 |
| Human platelet activating factor “acetylhydrolase,” brain | U72342 |
| “isoform,” 45 kDa subunit (LIS1) gene | |
| Homo sapiens ERK activator kinase (MEK2) mRNA | L11285 |
| Human 20-kDa myosin light chain (MLC-2) “mRNA,” | J02854 |
| complete cds | |
| H. sapiens lysosomal acid phosphatase gene (EC 3.1.3.2) | X15525 |
| Exon 1 (and joined CDS). | |
| Human mRNA for matrix Gla protein | X53331 |
| H. sapiens mRNA for diacylglycerol kinase | X62535 |
| Human heat shock protein (hsp 70) gene, complete cds. | M11717 |
| Human TRPM-2 protein gene | M63379 |

a Dukes B stag gene is selected individually from any gene comprising a sequence as identified below

| | | |
|---|---|---|
| RC_D59847_at | PPAPP | proSAAS; granin-like neuroen- |
| | | docrine peptide precursor |
| RC_F05038_at | PPAPP | polyamine modulated factor-1; |
| | | polyamine modulated factor 1 |
| RC_N41059_at | PPAPP | chrom 3 |
| RC_T23460_at | PPAPP | chrom 3; IFNAR2 21q22.11 |
| RC_W42789_at | PPAPP | chrom 8 AF268037 C8ORF4 |
| | | protein (C8ORF4) chrom 8 |
| | | ORF |
| RC_AA460017_i_{—} | PPAPP | BAC clone chrom 16 |
| at | | |
| RC_AA482127_at | PPAPP | KIAA1142 protein |
| RC_AA504806_at | PPAPP | chrom 2 AF052107 clone |
| | | 23620 mRNA sequence |
| RC_T90037_at | PPPPP | unnamed protein product, |
| | | chrom 4 |
| RC_AA432130_at | PPPPP | KIAA0867 protein, chrom 12 |

or any gene comprising a sequence as identified below

| | |
|---|---|
| Human gene for mitochondrial acetoacetyl-CoA thiolase | D10511 |
| Human mRNA for transcription factor “AREB6,” complete | D15050 |
| cds | |
| Human mRNA for KIAA0248 “gene,” partial cds | D87435 |
| Homo sapiens (clone CC6) NADH-ubiquinone oxidoreduc- | L04490 |
| tase subunit “mRNA,” 3′ end cds | |
| Human phosphoglucomutase 1 (PGM1) “mRNA,” com- | M83088 |
| plete cds | |
| Homo sapiens guanylin “mRNA,” complete cds | M97496 |
| “Human trans-Golgi p230 ”“mRNA,”“ complete cds” | U41740 |
| H. sapiens mRNA for vacuolar proton “ATPase,” subunit D | X71490 |
| H. sapiens mRNA for 3-hydroxy-3-methylglutaryl coen- | X83618 |
| zyme A synthase | |
| Human mRNA for KIAA0018 “gene,” complete cds | D13643 |
| “Mucin ”“1,”“ ”“Epithelial,”“ Alt. Splice 9” | HG371- |
| | HT26388 |
| H. sapiens mRNA for L-3-hydroxyacyl-CoA dehydrogenase | X96752 |

a Dukes C stage gene is selected individually from any gene comprising a sequence as identified below

| | | |
|---|---|---|
| RC_N30231_at | PPPAP | Lsm4 protein; U6 snRNA- |
| | | associated Sm-like protein |
| | | LSm4; glycine-rich protein |
| RC_W73790_f_at | PPPAP | immunoglobulin-related pro- |
| | | tein 14.1; lambda L-chain C |
| | | region; omega protein, chrom |
| | | 22 |
| RC_AA412184_at | PPPAP | chrom 1p36; d89060 dolichyl- |
| | | diphosphooligosaccharide- |
| | | protein glycosyltransferase |
| RC_AA521303_at | PPPAP | methionine adenosyltransfer- |
| | | ase regulatory beta subunit; |
| | | dTDP-4-keto-6-deoxy-D- |
| | | glucose 4-reductase, chrom 5 |
| RC_AA461174_at | PPPPP | 8p21.3-p22 AB020860 anti- |
| | | oncogene |
| AA393432_s_at | PPPPP | chrom 2, Unknown; unnamed |
| | | protein product AAD20029 |

or any gene comprising a sequence as identified below

| | |
|---|---|
| Homo sapiens colon mucosa-associated (DRA) | L02785 |
| “mRNA,” complete cds | |
| Human Ig J chain gene | M12759 |
| Human selenium-binding protein (hSBP) “mRNA,” | U29091 |
| complete cds. /gb = U29091 /ntype = RNA | |
| H. sapiens mRNA for sigma 3B protein | X99459 |
| Human ERK1 mRNA for protein serine/threonine kin- | X60188 |
| ase | |
| Human mRNA for mitochondrial 3-oxoacyl-CoA “thio- | D16294 |
| lase,” complete cds | |
| “Biliary ”“Glycoprotein,”“ Alt. Splice ”“5,”“ A” | HG2850- |
| | HT4814 |
| Human AQP3 gene for aquaporine 3 (water “channel),” | AB001325 |
| partail cds | |
| Human CD14 mRNA for myelid cell-specific leucine-rich | X13334 |
| glycoprotein | |
| Human thioredoxin “mRNA,” nuclear gene encoding mito- | U78678 |
| chondrial “protein,” complete cds | |
| Human mitochondrial ATPase coupling factor 6 subunit | M37104 |
| (ATP5A) “mRNA,” complete cds | |
| “Human MHC class II HLA-DP light chain ”“mRNA,”“ com- | M57466 |
| plete cds” | |
| Human mRNA for early growth response protein 1 | X52541 |
| (hEGR1) | |
| Human mRNA for mitochondrial 3-ketoacyl-CoA thiolase | D16481 |
| beta-subunit of trifunctional “protein,” complete cds | |
| Homo sapiens laminin-related protein (LamA3) “mRNA,” | L34155 |
| complete cds | |
| H. sapiens mRNA for selenoprotein P | Z11793 |
| Human hkf-1 “mRNA,” complete cds | D76444 |
| Homo sapiens nuclear domain 10 protein (ndp52) “mRNA,” | U22897 |
| complete cds | |
| Human X104 “mRNA,” complete cds | L27476 |
| H. sapiens cDNA for RFG | X77548 |
| H. sapiens mRNA for Progression Associated Protein | Y07909 |
| Human liver “2,4-dienoyl-CoA” reductase “mRNA,” com- | U49352 |
| plete cds | |
| Human A33 antigen precursor “mRNA,” complete cds | U79725 |
| H. sapiens pS2 protein gene | X52003 |
| Human RASF-A PLA2 “mRNA,” complete cds | M22430 |
| Homo sapiens pstl mRNA for pancreatic secretory inhibitor | Y00705 |
| (expressed in neoplastic tissue). | |
| Human CO-029 | M35252 |

a Dukes D stage gene is selected individually from any gene comprising a sequence as identified below

| | | |
|---|---|---|
| RC_R72886_s_at | PPPPA | KIAA0422; adenylyl cyclase |
| | | type VI, chrom 12 |
| RC_AA026030_at | PPPPA | chrom 1 |
| RC_Z39006_at | PPPPA | hypothetical protein, chrom 17 |
| RC_AA435908_at | PPPPA | chrom 19; ac011491 clone and |
| | | 20 nt hom. RAB2, RAS onco- |
| | | gene family |
| RC_AA057829_s | PPPPA | growth-arrest-specific protein; |
| _at | | growth arrest-specific 6; AXL |
| | | stimulatory factor, chrom 13 |
| RC_R72087_at | PPPPA | chrom 5 EST; hom to chrom |
| | | 20 AL356652 clone |
| RC_H04242_at | PPPPA | ras related protein Rab5b; |
| | | RAB5B, member RAS onco- |
| | | gene family |
| RC_R97304_f_at | PPPPA | HLA-drb5; cell surface gly- |
| | | coprotein; MHC HLA-DR-beta |
| | | chain precursor chrom 6 |
| RC_N48609_at | PPPPA | chrom 11; AC004584 chrom |
| | | 17 |
| RC_W86850_f_at | PPPPA | chrom 22 ? X96924 mito- |
| | | chondrial citrate tranbsport |
| | | region |
| RC_AA130603_at | PPPPA | ak024908 clone |
| RC_AA479610_at | PPPPA | singleton ak025344 clone |
| RC_AA490593_i_{—} | PPPPA | chrom 17 ? Synaptobrevin2 |
| at | | (VAMP2) AF135372 |
| RC_AA054321_s | PPPPA | 6p21 HLA class i region; |
| _at | | AC004202 clone |
| RC_D60328_at | PPPPP | chrom 6, unknown; ring finger |
| | | protein 5 |
| RC_H96850_at | PPPPP | oligosaccharyltransferase |
| | | d89060 1p36.1 (also C-class) |
| RC_AA127444_at | PPPPP | chrom 1 no homology |
| RC_AA242824_at | PPPPP | chrom 11; ac005233 PAC |
| | | clone chrom 22 |
| AA405775_s_at | PPPPP | similar to CAA16821 |
| | | (PID: g3255952) |

or any gene comprising a sequence as identified below

| | |
|---|---|
| Human complement component C3 “mRNA,” alpha and | K02765 |
| beta “subunits,” complete cds | |
| H. sapiens mRNA for adenosine “triphosphatase,” cal- | Z69881 |
| cium | |
| Human skeletal muscle LIM-protein SLIM1 “mRNA,” com- | U60115 |
| plete cds | |
| Human platelet-derived growth factor receptor alpha | M21574 |
| (PDGFRA) “mRNA,” complete cds | |
| Human mRNA for KIAA0247 “gene,” complete cds | D87434 |
| Human mRNA for KIAA0171 “gene,” complete cds | D79993 |
| Human Down syndrome critical region protein (DSCR1) | U28833 |
| “mRNA,” complete cds | |
| Human Ki nuclear autoantigen “mRNA,” complete cds | U11292 |

Expression Patterns

The objects of the invention are achieved by providing one or more of the embodiments described below. In one embodiment a method is provided of determining an expression pattern of a cell sample preferably independent of the proportion of submucosal, muscle and connective tissue cells present. Expression is determined of one or more genes in a sample comprising cells, said genes being selected from the same genes as discussed above and shown in the tables of the Examples.

It is an object of the present that characteristic patterns of expression of genes can be used to characterize different types of tissue. Thus, for example gene expression patterns can be used to characterize stages and grades of colorectal tumors. Similarly, gene expression patterns can be sued to distinguish cells having a colorectal origin from other c lls. Moreover, gene expression of cells which routinely contaminate colorectal tumor biopsies has been identified, and such gene expression can be removed or subtracted from patterns obtained from colorectal biopsies. Further, the gene expression patterns of single-cell solutions of colorectal tumor cells have been found to be far freer of interfering expression of contaminating muscle, submucosal, and connective tissue cells that biopsy samples.

The one or more genes exclude genes which are expressed in the submucosal, muscle, and connective tissue. A pattern of expression is formed for the sample which is independent of the proportion of submucosal, muscle, and connective tissue cells in the sample.

In another aspect of the invention a method of determining an expression pattern of a cell sample is provided. Expression is determined of one or more genes in a sample comprising cells. A first pattern of expression is thereby formed for the sample. Genes which are expressed in submucosal, muscle, and connective tissue cells are removed from the first pattern of expression, forming a second pattern of expression which is independent of the proportion of submucosal, muscle, and connective tissue cells in the sample.

Another embodiment of the invention provides a method for determining an expression pattern of a colorectal mucosa or colorectal cancer cell. Expression is determined of one or more genes in a sample comprising colorectal mucosa or colorectal cancer cells; the expression determined forms a first pattern of expression. A second pattern of expression which was formed using the one or more genes and a sample comprising predominantly submucosal, muscle, and connective tissue cells, is subtracted from the first pattern of expression, forming a third pattern of expression. The third pattern of expression reflects expression of the colorectal mucosa or colorectal cancer cells independent of the proportion of submucosal, muscle, and connective tissue cells present in the sample.

Diagnosing

In another embodiment of the invention a method is provided of detecting an invasive tumor in a patient. A marker is detected in a sample of a body fluid. The body fluid is selected from the group consisting of blood, plasma, serum, faeces, mucus, sputum, cerebrospinal fluid and/or urine. The marker is an mRNA or protein expression product of a gene which is more prevalent in submucosal, muscle, and connective tissue than in the body fluid. An increased amount of the marker in the body fluid indicates a tumor which has become invasive in the patient.

In another aspect of the invention a method is provided for diagnosing a colorectal cancer. A first pattern of expression is determined of one or more genes in a colonic tissue sample suspected of being neoplastic. The first pattern of expression is compared to a second and third reference pattern of expression. The second pattern is of the one or more genes in normal colorectal mucosa and the third pattern is of the one or more genes in colorectal cancer. A first pattern of expression which is found to be more similar to the third pattern than the second indicates neoplasia of the colorectal tissue sample.

According to yet another aspect of the invention a method is provided for predicting outcome or prescribing treatment of a colorectal tumor. A first pattern of expression is determined of one or more genes in a colorectal tumor sample. The first pattern is compared to one or more reference patterns of expression determined for colorectal tumors at a grade between I and IV. The reference pattern which shares maximum similarity with the first pattern is identified. The outcome or treatment appropriate for the grade of tumor of the reference pattern with the maximum similarity is assigned to the colorecteal tumor sample.

In another embodiment of the invention a method is provided for determining grade of a colorecteal tumor. A first pattern of expression is determined of one or more genes in a colorectal tumor sample. The first pattern is compared to one or more reference patterns of expression determined for colorectal tumors at a grade between I and IV. The grade of the reference pattern with the maximum similarity is assigned to the colorecteal tumor sample.

Yet another embodiment of the invention provides a method to determine stage of a colorectal tumor as described above. A first pattern of expression is determined of one or more genes in a colorectal tumor sampl. The first pattern is compared to one or more reference patterns of expression determined for colorectal tumors at different stages. The reference pattern which shares maximum similarity with the first pattern is identified. The stage of the reference pattern with the maximum similarity is assigned to the colorecteal tumor sample.

In still another embodiment of the invention a method is provided for identifying a tissue sample as colo-rectal. A first pattern of expression is determined of one or more genes in a tissue sample. The first pattern is compared to a second pattern of expression determined obtained for normal mucosa cells. Similarity between the first and the second patterns suggests that the tissue sample is mucosa in its origin. This method being particularly useful when diagnosing metastasis possibly distant from its origin.

Another aspect of the invention is a method to aid in diagnosing, predicting outcome, or prescribing treatment of a colorectal cancer. A first pattern of expression is determined of one or more genes in a first colorectal tissue sample. A second pattern of expression is determined of the one or more genes in a second colorectal tissue sample. The first colorectal tissue sample is a normal colorectal mucosa sample or an earlier stage or lover grade of colorectal tumor than the second colorectal tissue sample. The first pattern of expression is compared to the second pattern of expression to identify a first set of genes which are increased in the second colorectal tissue sample relative to the first colorectal tissue sample and a second set of genes which are decreased in the second colorectal tissue sample relative to the first colorectal tissue sample. Those genes which are expressed in submucosal, muscle or connective tissue are removed from the first set of genes. Those genes which are not expressed in submucosal, muscle, or connective tissue are removed from the second set of genes.

Independence of Submucosal, Muscle and Connective Tissue

Since a biopsy of the tissue often contains more tissue material, than the tissue to be examined, such as connective tissue, when the tissue to be examined is epithelial or mucosa, the invention also relates to methods, wherein the expression pattern of the tissue is independent of the amount of connective tissue in the sample.

Biopsies contain epithelial cells that most often are the targets for the studies, and in addition many other cells that contaminate the epithelial cell fraction to a varying extent. The contaminants include histiocytes, endothelial cells, leukocytes, nerve cells, muscle cells etc. Micro dissection is the method of choice for DNA examination, but in case of expression studies this procedure is difficult due to RNA degradation during the procedure. The epithelium may be gently removed and the expression in the remaining submucosa and underlying connective tissue (the colon wall) monitored. Genes expressed at high or low levels in the colon wall should be interrogated when performing expression monitoring of the mucosa and tumors. A similar approach could be used for studies of epithelia in other organs.

Normal mucosa lining the colon lumen from colons for colon cancer was scraped off. Then biopsies were taken from the denuded submucosa and connective tissue, reaching approximately 5 mm into the colon wall, and immediately disintegrated in guanidinium isothiocyanate. Total RNA may be extracted, pooled, and poly(A)⁺ mRNA may be prepared from the pool followed by conversion to double-stranded cDNA and in vitro transcription into cRNA containing biotin-labeled CTP and UTP.

Genes that are expressed and genes that are not expressed in colon wall can both interfere with the interpretation of the expression in a biopsy, and should be interrogated when interpreting expression intensities in tumor biopsies, as the colon wall component of a biopsy varies in amount from biopsy to biopsy.

When having determined the pattern of genes expressed in colon wall components said pattern may be subtracted from a pattern obtained from the sample resulting in a third pattern related to the mucosa (epithelial) cells.

In another aspect of the invention a method is provided for determining an expression pattern of a colorectal tissue sample independent of the proportion of submucosal, muscle and connective tissue cells present. A single-cell suspension of disaggregated colorectal tumor cells is isolated from a colorectal tissue sample comprising colorectal tumor cells is isolated form a coloretal tissue sample comprising colorectal cells, submucosal cells, muscle cells, and connective tissue cells. A pattern of expression is thus formed for the sample which is independent of the proportion of submucosal, muscle, and connective tissue cells in the colorectal tissue sample.

Yet another method relates to elimination mRNA from colon wall components before determining the pattern, e.g. by filtration and/or affinity chromatography to remove mRNA related to the colon wall.

Detection

Working with human tumor material requires biopsies, and working with RNA requires freshly frozen or immediately processed biopsies. Apart from the cancer tissue, biopsies do inevitably contain many different cell types, such as cells present in the blood, connective and muscle tissue, endothelium etc. In the case of DNA studies, microdissection or laser capture are method of choice, however the time dependent degradation of RNA makes it difficult to perform manipulation of the tissue for more than a few minutes. Furthermore, studies of expressed sequences may be difficult on the few cells obtained via microdissection or laser capture, as these may have an expression pattern that deviates from the predominant pattern in a tumor due to large intratumoral heterogeneity.

In the present context high density expression arrays may be used to evaluate the impact of colorectal wall components in colorectal tumor biopsies, and tested preparation of single cell solutions as a means of eliminating the contaminants. The results of these evaluations permit us to design methods of evaluating colorectal samples without the interfering background noise caused by ubiquitous contaminating submucosal, muscle, and connective tissue cells. The evaluating assays of the invention may be of any type.

While high density expression arrays can be used, other techniques are also contemplated. These include other techniques for assaying for specific mRNA species, including RT-PCR and Northern Blotting, as well as techniques for assaying for particular protein products, such as ELISA, Western blotting, and enzyme assays. Gene expression patterns according to the present invention are determined by measuring any gene product of a particular gene, including mRNA and protein. A pattern may be for one or more gene.

RNA or protein can be isolated and assayed from a test sample using any techniques known in the art. They can for example be isolated from fresh or frozen biopsy, from formalin-fixed tissue, from body fluids, such as blood, plasma, serum, urine, or sputum.

The data provided of expression for submucosal, muscle, and connective tissue can be used in at least three ways to improve the quality of data for a tested sample. The genes identified in the data as expressed can be excluded from the testing or from the analysis. Alternatively, the intensity of expression of the genes expressed in the submucosal, muscle, and connective tissue can be subtracted from the intensity of expression determined for the tests tissue.

The data collected and disclosed here as “connective tissue” is presumed to contain both muscle and submucosal gene expression as well. Thus it represents the composite expression of these cell types which can typically contaminate a colorectal biopsy.

Detection of Expression

Expression of genes may in general be detected by either detecting mRNA from the cells and/or detecting expression products, such as peptides and proteins.

mRNA Detection

The detection of mRNA of the invention may be a tool for determining the developmental stage of a cell type may be definable by its pattern of expression of messenger RNA. For example, in particular stages of cells, high levels of ribosomal RNA are found whereas relatively low levels of other types of messenger RNAs may be found. Where a pattern is shown to be characteristic of a stage, a stage may be defined by that particular pattern of messenger RNA expression. The mRNA population is a good determinant of developmental stage, will be correlated with other structural features of the cell. In this manner, cells at specific developmental stages will be characterized by the intracellular environment, as well as the extracellular environment. The present invention also allows the combination of definitions based, in part, upon antigens and, in part, upon mRNA expression. In one embodiment, the two may be combined in a single incubation step. A particular incubation condition may be found which is compatible with both hybridization recognition and non-hybridization recognition molecules. Thus, e.g., an incubation condition may be selected which allows both specificity of antibody binding and specificity of nucleic acid hybridization. This allows simultaneous performance of both types of interactions on a single matrix. Again, where developmental mRNA patterns are correlated with structural features, or with probes which are able to hybridize to intracellular mRNA populations, a cell sorter may be used to sort specifically those cells having desired mRNA population patterns.

It is within the general scope of the present invention to provide methods for the detection of mRNA. Such methods often involve sample extraction, PCR amplification, nucleic acid fragmentation and labeling, extension reactions, transcription reactions and the like.

Sample Preparation

The nucleic acid (either genomic DNA or mRNA) may be isolated from the sample according to any of a number of methods well known to those of skill in the art. One of skill will appreciate that where alterations in the copy number of a gene are to be detected genomic DNA is preferably isolated. Conversely, where expression levels of a gene or genes are to be detected, preferably RNA (mRNA) is isolated.

Methods of isolating total mRNA are well known to those of skill in the art. In one embodiment, the total nucleic acid is isolated from a given sample using, for example, an acid guanidinium-phenol-chloroform extraction method and polyA.sup.+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

The sample may be from tissue and/or body fluids, as defined elsewhere herein. Before analyzing the sample, e.g., on an oligonucleotide array, it will often be desirable to perform one or more sample preparation operations upon the sample. Typically, these sample preparation operations will include such manipulations as extraction of intracellular material, e.g., nucleic acids from whole cell samples, viruses and the like, amplification of nucleic acids, fragmentation, transcription, labeling and/or extension reactions. One or more of these various operations may be readily incorporated into the device of the present invention.

DNA Extraction

DNA extraction may be relevant in case possible mutations in the genes are to be dtermined in addition to the determination of expression of the genes.

For those embodiments where whole cells, or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations, e.g., denaturation of contaminating (DNA binding) proteins, purification, filtration, desalting, and the like.

Liberation of nucleic acids from the sample cells, and denaturation of DNA binding proteins may generally be performed by physical or chemical methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins.

Alternatively, physical methods may be used to extract the nucleic acids and denature DNA binding proteins, such as physical protrusions within microchannels or sharp edged particles piercing cell membranes and extract their contents. Combinations of such structures with piezoelectric elements for agitation can provide suitable shear forces for lysis.

More traditional methods of cell extraction may also be used, e.g., employing a channel with restricted cross-sectional dimension which causes cell lysis when the sample is passed through the channel with sufficient flow pressure. Alternatively, cell extraction and denaturing of contaminating proteins may be carried out by applying an alternating electrical current to the sample. More specifically, the sample of cells is flowed through a microtubular array while an alternating electric current is applied across the fluid flow. Subjecting cells to ultrasonic agitation, or forcing cells through microgeometry apertures, thereby subjecting the cells to high shear stress resulting in rupture are also possible extraction methods.

Filtration

Following extraction, it will often be desirable to separate the nucleic acids from other elements of the crude extract, e.g., denatured proteins, cell membrane particles, salts, and the like. Removal of particulate matter is generally accomplished by filtration, flocculation or the like. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art, may also be readily incorporated into the devices of the present invention, and is commercially available from, e.g., Pharmacia and Sigma Chemical.

Alternatively, desalting methods may generally take advantage of the high electrophoretic mobility and negative of DNA compared to other elements. Electrophoretic methods may also be utilized in the purification of nucleic acids from other cell contaminants and debris. Upon application of an appropriate electric field, the nucleic acids present in the sample will migrate toward the positive electrode and become trapped on the capture membrane. Sample impurities remaining free of the membrane are then washed away by applying an appropriate fluid flow. Upon reversal of the voltage, the nucleic acids are released from the membrane in a substantially purer form. Further, coarse filters may also be overlaid on the barriers to avoid any fouling of the barriers by particulate matter, proteins or nucleic acids, thereby permitting repeated use.

Separation of Contaminants by Chromatography

In a similar aspect, the high electrophoretic mobility of nucleic acids with their negative charges, may be utilized to separate nucleic acids from contaminants by utilizing a short column of a gel or other appropriate matrix or gel which will slow or retard the flow of other contaminants while allowing the faster nucleic acids to pass.

This invention provides nucleic acid affinity matrices that bear a large number of different nucleic acid affinity ligands allowing the simultaneous selection and removal of a large number of preselected nucleic acids from the sample. Methods of producing such affinity matrices are also provided. In general the methods involve the steps of a) providing a nucleic acid amplification template array comprising a surface to which are attached at least 50 oligonucleotides having different nucleic acid sequences, and wherein each different oligonucleotide is localized in a predetermined region of said surface, the density of said oligonucleotides is greater than about 60 different oligonucleotides per 1 cm.sup.2, and all of said different oligonucleotides have an identical terminal 3′ nucleic acid sequence and an identical terminal 5′ nucleic acid sequence. b) amplifying said multiplicity of oligonucleotides to provide a pool of amplified nucleic acids; and c) attaching the pool of nucleic acids to a solid support.

For example, nucleic acid affinity chromatography is based on the tendency of complementary, single-stranded nucleic acids to form a double-stranded or duplex structure through complementary base pairing. A nucleic acid (either DNA or RNA) can easily be attached to a solid substrate (matrix) where it acts as an immobilized ligand that interacts with and forms duplexes with complementary nucleic acids present in a solution contacted to the immobilized ligand. Unbound components can be washed away from the bound complex to either provide a solution lacking the target molecules bound to the affinity column, or to provide the isolated target molecules themselves. The nucleic acids captured in a hybrid duplex can be separated and released from the affinity matrix by denaturation either through heat, adjustment of salt concentration, or the use of a destabilizing agent such as formamide, TWEEN.TM.-20 denaturing agent, or sodium dodecyl sulfate (SDS). Affinity columns (matrices) are typically used either to isolate a single nucleic acid typically by providing a single species of affinity ligand. Alternatively, affinity columns bearing a single affinity ligand (e.g. oligo dt columns) have been used to isolate a multiplicity of nucleic acids where the nucleic acids all share a common sequence (e.g. a polyA).

Affinity Matrices

The type of affinity matrix used depends on the purpose of the analysis. For example, where it is desired to analyze mRNA expression levels of particular genes in a complex nucleic acid sample (e.g., total mRNA) it is often desirable to eliminate nucleic acids produced by genes that are constitutively overexpressed and thereby tend to mask gene products expressed at characteristically lower levels. Thus, in one embodiment, the affinity matrix can be used to remove a number of preselected gene products (e.g., actin, GAPDH, etc.). This is accomplished by providing an affinity matrix bearing nucleic acid affinity ligands complementary to the gene products (e.g., mRNAs or nucleic acids derived therefrom) or to subsequences thereof. Hybridization of the nucleic acid sample to the affinity matrix will result in duplex formation between the affinity ligands and their target nucleic acids. Upon elution of the sample from the affinity matrix, the matrix will retain the duplexes nucleic acids leaving a sample depleted of the overexpressed target nucleic acids.

The affinity matrix can also be used to identify unknown mRNAs or cDNAs in a sample. Where the affinity matrix contains nucleic acids complementary to every known gene (e.g., in a cDNA library, DNA reverse transcribed from an mRNA, mRNA used directly or amplified, or polymerized from a DNA template) in a sample, capture of the known nucleic acids by the affinity matrix leaves a sample enriched for those nucleic acid sequences that are unknown. In effect, the affinity matrix is used to perform a subtractive hybridization to isolate unknown nucleic acid sequences. The remaining “unknown” sequences can then be purified and sequenced according to standard methods.

The affinity matrix can also be used to capture (isolate) and thereby purify unknown nucleic acid sequences. For example, an affinity matrix can be prepared that contains nucleic acid (affinity ligands) that are complementary to sequences not previously identified, or not previously known to be expressed in a particular nucleic acid sample. The sample is then hybridized to the affinity matrix and those sequences that are retained on the affinity matrix are “unknown” nucleic acids. The retained nucleic acids can be eluted from the matrix (e.g. at increased temperature, increased destabilizing agent concentration, or decreased salt) and the nucleic acids can then be sequenced according to standard methods.

Similarly, the affinity matrix can be used to efficiently capture (isolate) a number of known nucleic acid sequences. Again, the matrix is prepared bearing nucleic acids complementary to those nucleic acids it is desired to isolate. The sample is contacted to the matrix under conditions where the complementary nucleic acid sequences hybridize to the affinity ligands in the matrix. The non-hybridized material is washed off the matrix leaving the desired sequences bound. The hybrid duplexes are then denatured providing a pool of the isolated nucleic acids. The different nucleic acids in the pool can be subsequently separated according to standard methods (e.g. gel electrophoresis).

As indicated above the affinity matrices can be used to selectively remove nucleic acids from virtually any sample containing nucleic acids (e.g., in a cDNA library, DNA reverse transcribed from an mRNA, mRNA used directly or amplified, or polymerized from a DNA template, and so forth). The nucleic acids adhering to the column can be removed by washing with a low salt concentration buffer, a buffer containing a destabilizing agent such as formamide, or by elevating the column temperature.

In one particularly preferred embodiment, the affinity matrix can be used in a method to enrich a sample for unknown RNA sequences (e.g. expressed sequence tags (ESTs)). The method involves first providing an affinity matrix bearing a library of oligonucleotide probes specific to known RNA (e.g., EST) sequences. Then, RNA from undifferentiated and/or unactivated cells and RNA from differentiated or activated or pathological (e.g., transformed) or otherwise having a different metabolic state are separately hybridized against the affinity matrices to provide two pools of RNAs lacking the known RNA sequences.

In a preferred embodiment, the affinity matrix is packed into a columnar casing. The sample is then applied to the affinity matrix (e.g. injected onto a column or applied to a column by a pump such as a sampling pump driven by an autosampler). The affinity matrix (e.g. affinity column) bearing the sample is subjected to conditions under which the nucleic acid probes comprising the affinity matrix hybridize specifically with complementary target nucleic acids. Such conditions are accomplished by maintaining appropriate pH, salt and temperature conditions to facilitate hybridization as discussed above.

For a number of applications, it may be desirable to extract and separate messenger RNA from cells, cellular debris, and other contaminants. As such, the device of the present invention may, in some cases, include an mRNA purification chamber or channel. In general, such purification takes advantage of the poly-A tails on mRNA. In particular and as noted above, poly-T oligonucleotides may be immobilized within a chamber or channel of the device to serve as affinity ligands for mRNA. Poly-T oligonucleotides may be immobilized upon a solid support incorporated within the chamber or channel, or alternatively, may be immobilized upon th surface(s) of the chamber or channel itself. Immobilization of oligonucleotides on the surface of the chambers or channels may be carried out by methods described herein including, e.g., oxidation and silanation of the surface followed by standard DMT synthesis of the oligonucleotides.

In operation, the lysed sample is introduced to a high salt solution to increase the ionic strength for hybridization, whereupon the mRNA will hybridize to th immobilized poly-T. The mRNA bound to the immobilized poly-T oligonucleotides is then washed free in a low ionic strength buffer. The poy-T oligonucleotides may be immobiliized upon poroussurfaces, e.g., porous silicon, zeolites silica xerogels, scintered particles, or other solid supports.

Hybridization

Following sample preparation, the sample can be subjected to one or more different analysis operations. A variety of analysis operations may generally be performed, including size based analysis using, e.g., microcapillary electrophoresis, and/or sequence based analysis using, e.g., hybridization to an oligonucleotide array.

In the latter case, the nucleic acid sample may be probed using an array of oligonucleotide probes. Oligonucleotide arrays generally include a substrate having a large number of positionally distinct oligonucleotide probes attached to the substrate. These arrays may be produced using mechanical or light directed synthesis methods which incorporate a combination of photolithographic methods and solid phase oligonucleotide synthesis methods.

Light Directed Synthesis of Oligonucleotide Arrays

The basic strategy for light directed synthesis of oligonucleotide arrays is as follows. The surface of a solid support, modified with photosensitive protecting groups is illuminated through a photolithographic mask, yielding reactive hydroxyl groups in the illuminated regions. A selected nucleotide, typically in the form of a 3′-O-phosphoramidite-activated deoxynucleoside (protected at the 5′ hydroxyl with a photosensitive protecting group), is then presented to the surface and coupling occurs at the sites that were exposed to light. Following capping and oxidation, the substrate is rinsed and the surface is illuminated through a second mask, to expose additional hydroxyl groups for coupling. A second selected nucleotide (e.g., 5′-protected, 3′-O-phosphoramidite-activated deoxynucleoside) is presented to the surface. The selective deprotection and coupling cycles are repeated until the desired set of products is obtained. Since photolithography is used, the process can be readily miniaturized to generate high density arrays of oligonucleotide probes. Furthermore, the sequence of the oligonucleotides at each site is known. See, Pease, et al. Mechanical synthesis methods are similar to the light directed methods except involving mechanical direction of fluids for deprotection and addition in the synthesis steps.

For some embodiments, oligonucleotide arrays may be prepared having all possible probes of a given length. The hybridization pattern of the target sequence on the array may be used to reconstruct the target DNA sequence. Hybridization analysis of large numbers of probes can be used to sequence long stretches of DNA or provide an oligonucleotide array which is specific and complementary to a particular nucleic acid sequence. For example, in particularly preferred aspects, the oligonucleotide array will contain oligonucleotide probes which are complementary to specific target sequences, and individual or multiple mutations of these. Such arrays are particularly useful in the diagnosis of specific disorders which are characterized by the presence of a particular nucleic acid sequence.

Following sample collection and nucleic acid extraction, the nucleic acid portion of the sample is typically subjected to one or more preparative reactions. These preparative reactions include in vitro transcription, labeling, fragmentation, amplification and other reactions. Nucleic acid amplification increases the number of copies of the target nucleic acid sequence of interest. A variety of amplification methods are suitable for use in the methods and device of the present invention, including for example, the polymerase chain reaction method or (PCR), the ligase chain reaction (LCR), self sustained sequence replication (3SR), and nucleic acid based sequence amplification (NASBA).

The latter two amplification methods involve isothermal reactions based on isothermal transcription, which produce both single stranded RNA (ssRNA) and double stranded DNA (dsDNA) as the amplification products in a ratio of approximately 30 or 100 to 1, respectively. As a result, where these latter methods are employed, sequence analysis may be carried out using either type of substrate, i.e., complementary to either DNA or RNA.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids.

PCR

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes sp cific to the internal standard for quantification of the amplified nucleic acid.

Thus, in one embodiment, this invention provides for a method of optimizing a probe set for detection of a particular gene. Generally, this method involves providing a high density array containing a multiplicity of probes of one or more particular length(s) that are complementary to subsequences of the mRNA transcribed by the target gene. In one embodiment the high density array may contain every probe of a particular length that is complementary to a particular mRNA. The probes of the high density array are then hybridized with their target nucleic acid alone and then hybridized with a high complexity, high concentration nucleic acid sample that does not contain the targets complementary to the probes. Thus, for example, where the target nucleic acid is an RNA, the probes are first hybridized with their target nucleic acid alone and then hybridized with RNA made from a cDNA library (e.g., reverse transcribed polyA.sup.+mRNA) where the sense of the hybridized RNA is opposite that of the target nucleic acid (to insure that the high complexity sample does not contain targets for the probes). Those probes that show a strong hybridization signal with their target and little or no cross-hybridization with the high complexity sample are preferred probes for use in the high density arrays of this invention.

PCR amplification generally involves the use of one strand of the target nucleic acid sequence as a template for producing a large number of complements to that sequence. Generally, two primer sequences complementary to different ends of a segment of the complementary strands of the target sequence hybridize with their respective strands of the target sequence, and in the presence of polymerase enzymes and nucleoside triphosphates, the primers are extended along the target sequence. The extensions are melted from the target sequence and the process is repeated, this time with the additional copies of the target sequence synthesized in the preceding steps. PCR amplification typically involves repeated cycles of denaturation, hybridization and extension reactions to produce sufficient amounts of the target nucleic acid. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which ach primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In PCR methods, strand separation is normally achieved by heating the reaction to a sufficiently high temperature for a sufficient time to cause the denaturation of the duplex but not to cause an irreversible denaturation of the polymerase. Typical heat denaturation involves temperatures ranging from about 80.degree. C. to 105.degree. C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity.

In addition to PCR and IVT reactions, the methods and devices of the present invention are also applicable to a number of other reaction types, e.g., reverse transcription, nick translation, and the like.

Labelling Before Hybridization

The nucleic acids in a sample will generally be labeled to facilitate detection in subsequent steps. Labeling may be carried out during the amplification, in vitro transcription or nick translation processes. In particular, amplification, in vitro transcription or nick translation may incorporate a label into the amplified or transcribed sequence, either through the use of labeled primers or the incorporation of labeled dNTPs into the amplified sequence.

Hybridization between the sample nucleic acid and the oligonucleotide probes upon the array is then detected, using, e.g., epifluorescence confocal microscopy. Typically, sample is mixed during hybridization to enhance hybridization of nucleic acids in the sample to nucleoc acid probes on the array.

Labelling After Hybridization

In some cases, hybridized oligonucleotides may be labeled following hybridization. For example, where biotin labeled dNTPs are used in, e.g., amplification or transcription, streptavidin linked reporter groups may be used to label hybridized complexes. Such operations are readily integratable into the systems of the present invention. Alternatively, the nucleic acids in the sample may be labeled following amplification. Post amplification labeling typically involves the covalent attachment of a particular detectable group upon the amplified sequences. Suitable labels or detectable groups include a variety of fluorescent or radioactive labeling groups well known in the art. These labels may also be coupled to the sequences using methods that are well known in the art.

Methods for detection depend upon the label selected. A fluorescent label is preferred because of its extreme sensitivity and simplicity. Standard labeling procedures are used to determine the positions where interactions between a sequence and a reagent take place. For example, if a target sequence is labeled and exposed to a matrix of different probes, only those locations where probes do interact with the target will exhibit any signal. Alternatively, other methods may be used to scan the matrix to determine where interaction takes place. Of course, the spectrum of interactions may be determined in a temporal manner by repeated scans of interactions which occur at each of a multiplicity of conditions. However, instead of testing each individual interaction separately, a multiplicity of sequence interactions may be simultaneously determined on a matrix.

Means of detecting labeled target (sample) nucleic acids hybridized to the probes of the high density array are known to those of skill in the art. Thus, for example, where a colorimetric label is used, simple visualization of the label is sufficient. Where a radioactive labeled probe is used, detection of the radiation (e.g with photographic film or a solid state detector) is sufficient.

In a preferred embodiment, however, the target nucleic acids are labeled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

The target polynucleotide may be labeled by any of a number of convenient detectable markers. A fluorescent label is preferred because it provides a very strong signal with low background. It is also optically detectable at high resolution and sensitivity through a quick scanning procedure. Other potential labeling moieties include, radioisotopes, chemiluminescent compounds, labeled binding proteins, heavy metal atoms, spectroscopic markers, magnetic labels, and linked enzymes. Another method for labeling may bypass any label of the target sequence. The target may be exposed to the probes, and a double strand hybrid is formed at those positions only. Addition of a double strand specific reagent will detect where hybridization takes place. An intercalative dye such as ethidium bromide may be used as long as the probes themselves do not fold back on themselves to a significant extent forming hairpin loops. However, the length of the hairpin loops in short oligonucleotide probes would typically be insufficient to form a stable duplex.

Suitable chromogens will include molecules and compounds which absorb light in a distinctive range of wavelengths so that a color may be observed, or emit light when irradiated with radiation of a particular wave length or wave length range, e.g., fluorescers. Biliproteins, e.g., phycoerythrin, may also serve as labels.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers may be employed either by themselves or in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bisbenzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene; 4-acetamido-4-isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4-(3′pyrenyl)butyrate; d-3-aminodesoxy-equilenin; 12-(9′-anthroyl)stearate; 2-methylanthracene; 9-vinylanthracene; 2,2′-(vinylene-p-phenylene)bisbenzoxazole; p-bis>2-(4-methyl-5-phenyl-oxazolyl)!benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N->p-(2-benzimidazolyl)-phenyl!maleimide; N-(4-fluoranthyl)maleimide; bis(homovanillic acid); resazarin; 4-chloro-7-nitro-2,1,3-benzooxadiazole; merocyanine 540; resomufin; rose bengal; and 2,4-diphenyl-3(2H)-furanone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye may differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal may also be provided by chemiluminescent and bioluminescent sources. Chemiluminescent sources include a compound which becomes electronically excited by a chemical reaction and may then emit light which serves as the detectible signal or donates energy to a fluorescent acceptor. A diverse number of families of compounds have been found to provide chemiluminescence under a variety of conditions. One family of compounds is 2,3-dihydro-1,-4-phthalazinedione. The most popular compound is luminol, which is the 5-amino compound. Other members of the family include the 5-amino-6,7,8-trimethoxy- and the dimethylamino>ca!benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence may also be obtained with oxalates, usually oxalyl active esters, e.g., p-nitrophenyl and a peroxide, e.g., hydrogen peroxide, under basic conditions. Alternatively, luciferins may be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

Fragmentation

In addition, amplified sequences may be subjected to other post amplification treatments. For example, in some cases, it may be desirable to fragment the sequence prior to hybridization with an oligonucleotide array, in order to provide segments which are more readily accessible to the probes, which avoid looping and/or hybridization to multiple probes. Fragmentation of the nucleic acids may generally be carried out by physical, chemical or enzymatic methods that are known in the art.

Sample Analysis

Following the various sample preparation operations, the sample will generally be subjected to one or more analysis operations. Particularly preferred analysis operations include, e.g., sequence based analyses using an oligonucleotide array and/or size based analyses using, e.g., microcapillary array electrophoresis.

Capillary Electrophoresis

In some embodiments, it may be desirable to provide an additional, or alternative means for analyzing the nucleic acids from the sample

Microcapillary array electrophoresis generally involves the use of a thin capillary or channel which may or may not be filled with a particular separation medium. Electrophoresis of a sample through the capillary provides a size based separation profile for the sample. Microcapillary array electrophoresis generally provides a rapid method for size based sequencing, PCR product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Gel matrices may be introduced and polymerized within the capillary channel. However, in some cases, this may result in entrapment of bubbles within the channels which can interfere with sample separations. Accordingly, it is often desirable to place a preformed separation matrix within the capillary channel(s), prior to mating the planar elements of the capillary portion. Fixing the two parts, e.g., through sonic welding, permanently fixes the matrix within the channel. Polymerization outside of the channels helps to ensure that no bubbles are formed. Further, the pressure of the welding process helps to ensure a void-free system.

In addition to its use in nucleic acid “fingerprinting” and other sized based analyses, the capillary arrays may also be used in sequencing applications. In particular, gel based sequencing techniques may be readily adapted for capillary array electrophoresis.

Expression Products

In addition to detection of mRNA or as the sole detection method expression products from the genes discussed above may be detected as indications of the biological condition of the tissue. Expression products may be detected in either the tissue sample as such, or in a body fluid sample, such as blood, serum, plasma, faeces, mucus, sputum, cerebrospinal fluid, and/or urine of the individual.

The expression products, peptides and proteins, may be detected by any suitable technique known to the person skilled in the art.

In a preferred embodiment the expression products are detected by means of specific antibodies directed to the various expression products, such as immunofluorescent and/or immunohistochemical staining of the tissue.

Immunohistochemical localization of expressed proteins may be carried out by immunostaining of tissue sections from the single tumors to determine which cells expressed the protein encoded by the transcript in question. The transcript levels were used to select a group of proteins supposed to show variation from sample to sample, making possible a rough correlation between level of protein detected and intensity of the transcript on the microarray.

For example sections were cut from paraffin-embedded tissue blocks, mounted, and deparaffinized by incubation at 80 C° for 10 min, followed by immersion in heated oil at 60 C for 10 min (Estisol 312, Estichem A/S, Denmark) and rehydration. Antigen retrieval is achieved in TEG (TrisEDTA-Glycerol) buffer using microwaves at 900 W. The tissue sections cooled in the buffer for 15 min before a brief rinse in tap water. Endogenous peroxidase activity is blocked by incubating the sections with 1% H202 for 20 min, followed by three rinses in tap water, 1 min each. The sections are then soaked in PBS buffer for 2 min. The next steps are modifi d from the descriptions given by Oncogene Science Inc., in the Mouse Immunohistochemistry Detection System, XHCO1 (UniTect, Uniondale, N.Y., USA). Briefly, the tissue sections are incubated overnight at 4 C with primary antibody (against beta-2 microglobulin (Dako), cytokeratin 8, cystatin-C (both from Europa, US), junB, CD59, E-cadherin, apo-E, cathepsin E, vimentin, IGFII (all from Santa Cruz), followed by three rinses in PBS buffer for 5 min each. Afterwards, the sections are incubated with biotinylated secondary antibody for 30 min, rinsed three times with PBS buffer and subsequently incubated with ABC (avidin-biotinlylated horseradish peroxidase complex) for 30 min, followed by three rinses in PBS buffer.

Staining is performed by incubation with AEC (3-amino-ethylcarbazole) for 10 min. The tissue sections are counter stained with Mayers hematoxylin, washed in tap water for 5 min. and mounted with glycerol-gelatin. Positive and negative controls may be included in each staining round with all antibodies.

In yet another embodiment the expression products may be detected by means of conventional enzyme assays, such as ELISA methods.

Furthermore, the expression products may be detected by means of peptide/protein chips capable of specifically binding the peptides and/or proteins assessed. Thereby an expression pattern may be obtained.

Assay

Thus, in a further aspect the invention relates to an assay for determining an expression pattern of a colon and/or rectum cell, comprising at least a first marker and/or a second marker, wherein the first marker is capable of detecting a gene from a first gene group as defined above, and the second marker is capable of detecting a gene from a second gene group as defined above.

In a preferred embodiment the assay comprises at least two markers for each gene group.

correlating the first expression level and the second expression level to a standard level of the assessed genes to determine the presence or absence of a biological condition in the animal tissue.

The marker (s) are preferably specifically detecting a gene as identified herein, in particular the genes of the tables in the examples and as discussed above.

As discussed above the marker may be any nucleotide probe, such as a DNA, RNA, PNA, or LNA probe capable of hybridising to mRNA indicative of the expression level. The hybridisation conditions are preferably as described below for probes.

In another embodiment the marker is an antibody capable of specifically binding the expression product in question.

Detection

Patterns can be compared manually by a person or by a computer or other machine. An algorithm can be used to detect similarities and differences. The algorithm may score and compare, for example, the genes which are expressed and the genes which are not expressed. Alternatively, the algorithm may look for changes in intensity of expression of a particular gene and score changes in intensity between two samples. Similarities may be determined on the basis of genes which are expressed in both samples and genes which are not expressed in both samples or on the basis of genes whose intensity of expression are numerically similar.

Generally, the detection operation will be performed using a reader device external to the diagnostic device. However, it may be desirable in some cases, to incorporate the data gathering operation into the diagnostic device itself.

The detection apparatus may be a fluorescence detector, or a spectroscopic detector, or another detector.

Although hybridization is one type of specific interaction which is clearly useful for use in this mapping embodiment, antibody reagents may also be very useful.

Data Gathering and Analysis

Gathering data from the various analysis operations, e.g., oligonucleotide and/or microcapillary arrays, will typically b carried out using methods known in the art. For example, the arrays may be scanned using lasers to excite fluorescently labeled targets that have hybridized to regions of probe arrays mentioned above, which can then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection.

Following the data gathering operation, the data will typically be reported to a data analysis operation. To facilitate the sample analysis operation, the data obtained by the reader from the device will typically be analyzed using a digital computer. Typically, the computer will be appropriately programmed for receipt and storage of the data from the device, as well as for analysis and reporting of the data gathered, i.e., interpreting fluorescence data to determine the sequence of hybridizing probes, normalization of background and single base mismatch hybridizations, ordering of sequence data in SBH applications, and the like.

It is an object of the present invention to provide a biological sample which may be classified or characterized by analyzing the pattern of specific interactions mentioned above. This may be applicable to a cell or tissue type, to the messenger RNA population expressed by a cell to the genetic content of a cell, or to virtually any sample which can be classified and/or identified by its combination of specific molecular properties.

Pharmaceutical Composition

The invention also relates to a pharmaceutical composition for treating the bioligical condition, such as colorectal tumors.

In one embodiment the pharmaceutical composition comprises one or more of the peptides being expression products as defined above. In a preferred embodiment, the peptides are bound to carriers. The peptides may suitably be coupled to a polymer carrier, for example a protein carrier, such as BSA. Such formulations are well-known to the person skilled in the art.

The peptides may be suppressor peptides normally lost or decreased in tumor tissue administered in order to stabilise tumors towards a less malignant stage. In another embodiment the peptides are onco-peptides capable of eliciting an immune response towards the tumor cells.

In another embodiment the pharmaceutical composition comprises genetic material, either genetic material for substitution therapy, or for suppressing therapy as discussed below.

In a third embodiment the pharmaceutical composition comprises at least one antibody produced as described above.

In the present context the term pharmaceutical composition is used synonymously with the term medicament. The medicament of the invention comprises an effective amount of one or more of the compounds as defined above, or a composition as defined above in combination with pharmaceutically acceptable additives. Such medicament may suitably be formulated for oral, percutaneous, intramuscular, intravenous, intracranial, intrathecal, intracerebroventricular, intranasal or pulmonal administration. For most indications a localised or substantially localised application is preferred.

Strategies in formulation development of medicaments and compositions based on the compounds of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing AG, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which nhance the effectiven ss or transportation of the preparation.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosag ranges are of the order of several hundred μg active ingredient per administration with a preferred range of from about 0.1 μg to 1000 μg, such as in the range of from about 1 μg to 300 μg, and especially in the range of from about 10 μg to 50 μg. Administration may be performed once or may be followed by subsequent administrations. The dosage will also depend on the route of administration and will vary with the age and weight of the subject to be treated. A preferred dosis would be in the interval 30 mg to 70 mg per 70 kg body weight.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promote delivery of the active substance to its target.

In many instances, it will be necessary to administrate the formulation multiple times. Administration may be a continuous infusion, such as intraventricular infusion or administration in more doses such as more times a day, daily, more times a week, weekly, etc.

Vaccines

In a further embodiment the present invention relates to a vaccine for the prophylaxis or treatment of a biological condition comprising at least one expression product from at least one gene said gene being expressed as defined above.

The term vaccines is used with its normal meaning, i.e preparations of immunogenic material for administration to induce in the recipient an immunity to infection or intoxication by a given infecting agent. Vaccines may be administered by intravenous injection or through oral, nasal and/or mucosal administration. Vaccines may be either simple vaccines prepared from one species of expression products, such as proteins or peptides, or a variety of expression products, or they may be mixed vaccines containing two or more simple vaccines. They are prepared in such a manner as not to destroy the immunogenic material, although the methods of preparation vary, depending on the vaccine.

The enhanced immune response achieved according to the invention can be attributable to e.g. an enhanced increase in the level of immunoglobulins or in the level of T-cells including cytotoxic T-cells will result in immunisation of at least 50% of individuals exposed to said immunogenic composition or vaccine, such as at least 55%, for example at least 60%, such as at least 65%, for example at least 70%, for example at least 75%, such as at least 80%, for example at least 85%, such as at least 90%, for example at least 92%, such as at least 94%, for example at least 96%, such as at least 97%, for example at least 98%, such as at least 98.5%, for example at least 99%, for example at least 99.5% of the individuals exposed to said immunogenic composition or vaccine are immunised.

Compositions according to the invention may also comprise any carrier and/or adjuvant known in the art including functional equivalents thereof. Functionally equivalent carriers are capable of presenting the same immunogenic determinant in essentially the same steric conformation when used under similar conditions. Functionally equivalent adjuvants are capable of providing similar increases in the efficacy of the composition when used under similar conditions.

Therapy

The invention further relates to a method of treating individuals suffering from the biological condition in question, in particular for treating a colorectal tumor.

In one embodiment the invention relates to a method of substitution therapy, ie. administration of genetic material generally expressed in normal cells, but lost or decreased in biological condition cells (tumor suppressors). Thus, the invention relates to a method for reducing cell tumorigenicity of a cell, said method comprising

obtaining at least one gene selected from genes being expressed in an amount twofold higher in normal cells than the amount expressed in said tumor cell (tumor suppressors),

introducing said at least one gene into the tumor cell in a manner allowing expression of said gene(s).

The at least one gene is preferably selected individually from genes comprising a sequence as identified below

| | |
|---|---|
| RC_H04768_at | chrom 15 no homology |
| RC_Z39652_at | Y14593 APM-1 gene adipocyte-specific se- |
| | cretory protein; chrom 1q21.3-q23 |
| RC_H30270_at | chrom 18 PAAAA in colon & bladder no |
| | homology |
| RC_T47089_s_at | tenascin-X; tenascin-X precursor; unidenti- |
| | fied protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homolo- |
| | gy to mouse Pcbp1 - poly(rC)-binding |
| | protein 1 |
| AA319615_at | secretory carrier membrane protein; secre- |
| | tory carrier membrane protein 2; chrom 15 |

and from

| | |
|---|---|
| “Human chromogranin A” “mRNA,” “complete cds” | J03915 |
| Human adipsin/complement factor D “mRNA,” com- | M84526 |
| plete cds | |
| Homo sapiens MLC-1V/Sb isoform gene | M24248 |
| Human aminopeptidase N/CD13 mRNA encoding | M22324 |
| aminopeptidase “N,” complete cds | |
| H. sapiens MT-1I mRNA | X76717 |
| H. sapiens GCAP-II gene | Z70295 |
| Human somatostatin I gene and flanks | J00306 |
| Human YMP “mRNA,” complete cds | U52101 |
| H. sapiens mRNA for beta subunit of epithelial amiloride- | X87159 |
| sensitive sodium channel | |
| Human K12 protein precursor “mRNA,” complete cds | U77643 |
| Human sulfate transporter (DTD) “mRNA,” complete cds | U14528 |
| Human transcription factor hGATA-6 “mRNA,” complete | U66075 |
| cds. | |
| H. sapiens SCAD “gene,” exon 1 and joining features | Z80345 |
| Human S-lac lectin L-14-II (LGALS2) gene | M87860 |
| Human mRNA for protein tyrosine phosphatase | D15049 |
| H. sapiens mRNA for tetranectin | X64559 |
| Human 11 kd protein “mRNA,” complete cds | U28249 |
| Human anti-mullerian hormone type II receptor precursor | U29700 |
| “gene,” complete cds | |
| Human heparin binding protein (HBp17) “mRNA,” complete | M60047 |
| cds | |
| Human ADP-ribosylation factor (hARF6) “mRNA,” complete | M57763 |
| cds | |
| beta -ADD = adducin beta subunit 63 kda isoform/membrane | S81083 |
| skeleton protein, beta -ADD = adducin beta subunit 63 kda | |
| isoform/membrane skeleton protein {alternatively spliced, | |
| exon 10 to 13 region} [human, Genomic, 1851 nt, segment | |
| 3 of 3]. | |
| Zinc Finger Protein Znf155 | HG4243- |
| | HT4513 |
| Human glucagon “mRNA,” complete cds | J04040 |
| H. sapiens mRNA for hair “keratin,” hHb5 | X99140 |
| Human tubulin-folding cofactor E “mRNA,” complete cds | U61232 |
| Human integrin alpha-3 chain “mRNA,” complete cds | M59911 |
| Human NACP gene | U46901 |
| H. sapiens mRNA for flavin-containing monooxygenase 5 | Z47553 |
| (FMO5) | |
| Human mRNA for ATF-a transcription factor | X52943 |
| H. sapiens intestinal VIP receptor related protein mRNA | X77777 |

In a preferred embodiment at least two different genes are introduced into the tumor cell.

In another aspect the invention relates to a therapy whereby genes generally correlated to disease are inhibited by one or more of the following methods:

A method for reducing cell tumorigenicity of a cell, said method comprising

obtaining at least one nucleotide probe capable of hybridising with at least one gene of a tumor cell, said at least one gene being selected from genes being expressed in an amount at least one-fold lower in normal cells than the amount expressed in said tumor cell, and

introducing said at least one nucleotide probe into the tumor cell in a manner allowing the probe to hybridise to the at least one gene, thereby inhibiting expression of said at least one gene. This method is preferably based on anti-sense technology, whereby the hybridisation of said probe to the gene leads to a down-regulation of said gene.

The down-regulation may of course also be based on a probe capable of hybridising to regulatory components of the genes in question, such as promoters.

The probes are preferably selected from probes capable of hybridising to a nucleotide sequence comprising a sequence as identified below

| | | |
|---|---|---|
| RC_AA609013_s_at | APPPP | microsomal dipeptidase (also |
| | | on 6.8 k); chrom 16 |
| RC_AA232508_at | APPPP | CGI-89 protein; unnamed |
| | | protein product; hypothetical |
| | | protein |
| RC_AA428964_at | APPPP | serine protease-like protease; |
| | | serine protease homo- |
| | | log = NES1; normal epithelial |
| | | cell-specific 1 |
| RC_T52813_s_at | APPPP | dJ28O10.2 (G0S2 (PUTATIVE |
| | | LYMPHOCYTE G0/G1 |
| | | SWITCH PROTEIN 2; chrom 1 |
| RC_AA075642_at | APPPP | gp-340 variant protein; |
| | | DMBT1/8kb.2 protein |
| RC_AA007218_at | APPPP | chrom 13 no homology |
| RC_N33920_at | APPPP | ubiquitin-like protein FAT10; |
| | | diubiquitin; dJ271M21.6 (Diu- |
| | | biquitin); chrom 6 |
| RC_N71781_at | APPPP | KIAA1199 protein, chrom 15 |
| RC_R67275_s_at | APPPP | alpha-1 (type XI) collagen pre- |
| | | cursor; collagen, type XI, alpha |
| | | 1; collagen type XI alpha-1 |
| | | isoform A; chrom 1 |
| RC_W80763_at | APPPP | hypothetical protein; chrom 17 |
| RP_AA443793_at | APPPP | chrom 7p22 AC006028 BAC |
| | | clone |
| RC_AA034499_s_at | APPPP | ZNF198 protein; zinc finger |
| | | protein; FIM protein; Cys-rich |
| | | protein; zinc finger protein 198; |
| | | chrom 13 |
| RC_AA035482_at | APPPP | chrom 5; AK022505 clone; |
| | | CalcineurinB (weakly similar) |
| RC_AA024482_at | APPPP | hypothetical protein; unnamed |
| | | protein product; chrom 17 |
| RC_H93021_at | APPPP | chrom 2; XM_004890 pep- |
| | | tidylprolyl isomerase A (cy- |
| | | clophilin A) |
| RC_AA427737_at | APPPP | no homology |
| RC_AA417078_at | APPPP | chrom 7q31; AF017104 clone |
| M29873_s_at | APPPP | cytochrome P450-IIB (hIIB3); |
| | | 19q13.1-q13.2 |
| RC_H27498_f_at | AAPPP | |
| RC_T92363_s_at | AAPPP | |
| RC_N89910_at | AAAPP | |
| RC_W60516_at | AAAPP | |
| RC_AA219699_at | AAAPP | |
| RC_AA449450_at | AAAPP | |

Or from

| | |
|---|---|
| Homo sapiens (clones “MDP4,” MDP7) microsomal | J05257 |
| dipeptidase (MDP) “mRNA,” complete cds | |
| “Homo sapiens reg gene” “homologue,” “compl t | L08010 |
| cds” | |
| H. sapiens mRNA for prepro-alpha2(I) collagen | Z74616 |
| “Human S-adenosylhomocysteine hydrolase (AHCY)” | M61832 |
| “mRNA,” “complete cds” | |
| Transcription Fact r Iiia | HG4312- |
| | HT4582 |
| Human gene for melanoma growth stimulatory activity | X54489 |
| (MGSA) | |
| Human stromelysin-3 mRNA | X57766 |
| CDC25Hu2 = cdc25 + homolog “[human,” “mRNA,” | S78187 |
| 3118 nt] | |
| Human mRNA for cripto protein | X14253 |
| Human transformation-sensitive protein (IEF SSP 3521) | M86752 |
| “mRNA,” complete cds | |
| Human complement component 2 (C2) gene allele b | L09708 |
| H. sapiens mRNA for ITBA2 protein | X92896 |
| H. sapiens encoding CLA-1 mRNA | 222555 |
| “Human fibroblast growth factor receptor 4 (FGFR4)” | L03840 |
| “mRNA,” “complete cds” | |
| “”“Fibronectin,” “Alt. Splice 1” | HG3044- |
| | HT3742 |
| tyk2 | X54667 |
| Human mRNA for B-myb gene | X13293 |
| “Human phosphofructokinase (PFKM)” “mRNA,” | U24183 |
| “ complete cds” | |
| Human pre-B cell enhancing factor (PBEF) “mRNA,” com- | U02020 |
| plete cds | |
| Human SH2-containing inositol 5-phosphatase (hSHIP) | U57650 |
| “mRNA,” complete cds | |
| Human interleukin 8 (IL8) “gene,” complete cds | M28130 |
| “Human lamin B receptor (LBR)” “mRNA,” | L25931 |
| “complete cds” | |
| H. sapiens mRNA for protein tyrosine phosphatase | Z48541 |
| Human mRNA for unc-18 “homologue,” complete cds | D63851 |
| H. sapiens mRNA for Zn-alpha2-glycoprotein | X59766 |
| | Z25521 |
| “Human asparagine synthetase” “mRNA,” | M27396 |
| “complete cds” | |
| Human hepatitis delta antigen interacting protein A (dipA) | U63825 |
| “mRNA,” complete cds | |
| Human splicesomal protein (SAP 61) “mRNA,” complete | U08815 |
| cds | |
| Human protein kinase C-binding protein RACK7 “mRNA,” | U48251 |
| partial cds | |
| Human MAC30 “mRNA,” 3′ end | L19183 |
| Human thrombospondin 2 (THBS2) “mRNA,” complete cds | L12350 |
| “Human nicotinamide N-methyltransferase (NNMT)” | U08021 |
| “mRNA,” “complete cds” | |
| H. sapiens mRNA for type I interstitial collagenase | X54925 |
| Human cytochrome b561 gene | U29463 |
| Human H19 RNA “gene,” complete cds (spliced in sili- | M32053 |
| co) | |
| Human collagen type XVIII alpha 1 (COL18A1) “mRNA,” | L22548 |
| partial cds | |
| Human clone 23733 “mRNA,” complete cds. | U79274 |

In another embodiment the probes consists of the sequences identified above.

The hybridization may be tested in vitro at conditions corresponding to in vivo conditions. Typically, hybridization conditions are of low to moderate stringency. These conditions favour specific interactions between completely complementary sequences, but allow some non-specific interaction between less than perfectly matched sequences to occur as well. After hybridization, the nucleic acids can be “washed” under moderate or high conditions of stringency to dissociate duplexes that are bound together by some non-specific interaction (the nucleic acids that form these duplexes are thus not completely complementary).

As is known in the art, the optimal conditions for washing are determined empirically, often by gradually increasing the stringency. The parameters that can be changed to affect stringency include, primarily, temperature and salt concentration. In general, the lower the salt concentration and the higher the temperature, the higher the stringency. Washing can be initiated at a low temperature (for example, room temperature) using a solution containing a salt concentration that is equivalent to or lower than that of the hybridization solution. Subsequent washing can be carried out using progressively warmer solutions having the same salt concentration. As alternatives, the salt concentration can be lowered and the temperature maintained in the washing step, or the salt concentration can be lowered and the temperature increased. Additional parameters can also be altered. For example, use of a destabilizing agent, such as formamide, alters the stringency conditions.

In reactions where nucleic acids are hybridized, the conditions used to achieve a given level of stringency will vary. There is not one set of conditions, for example, that will allow duplexes to form between all nucleic acids that are 85% identical to one another; hybridization also depends on unique features of each nucleic acid. The length of the sequence, the composition of the sequence (for example, the content of purine-like nucleotides versus the content of pyrimidine-like nucleotides) and the type of nucleic acid (for example, DNA or RNA) affect hybridization. An additional consideration is whether one of the nucleic acids is immobilized (for example, on a filter).

An example of a progression from lower to higher stringency conditions is the following, where the salt content is given as the relative abundance of SSC (a salt solution containing sodium chloride and sodium citrate; 2×SSC is 10-fold more concentrated than 0.2×SSC). Nucleic acids are hybridized at 42° C. in 2×SSC/0.1% SDS (sodium dodecylsulfate; a detergent) and then washed in 0.2×SSC/0.1% SDS at room temperature (for conditions of low stringency); 0.2×SSC/0.1% SDS at 42° C. (for conditions of moderate stringency); and 0.1×SSC at 68° C. (for conditions of high stringency). Washing can be carried out using only one of the conditions given, or each of the conditions can be used (for example, washing for 10-15 minutes each in the order listed above). Any or all of the washes can be repeated. As mentioned above, optimal conditions will vary and can be determined empirically.

In another aspect a method of reducing tumoregeneicity relates to the use of antibodies against an expression product of a cell from the biological tissue. The antibodies may be produced by any suitable method, such as a method comprising the steps of

obtaining expression product(s) from at least one gene said gene being expressed as defined above for oncogenes,

immunising a mammal with said expression product(s) obtaining antibodies against the expression product.

Use

The methods described above may be used for producing an assay for diagnosing a biological condition in animal tissue, or for identification of the origin of a piece of tissue.

Furthermore, the invention relates to the use of a peptide as defined above for preparation of a pharmaceutical composition for the treatment of a biological condition in animal tissue.

Furthermore, the invention relates to the use of a gene as defined above for preparation of a pharmaceutical composition for the treatment of a biological condition in animal tissue.

Also, the invention relates to the use of a probe as defined above for preparation of a pharmaceutical composition for the treatment of a biological condition in animal tissue.

Gene Delivery Therapy

The genetic material discussed above for may be any of the described genes or functional parts thereof. The constructs may be introduced as a single DNA molecule encoding all of the genes, or different DNA molecules having one or more genes. The constructs may be introduced simultaneously or consecutively, each with the same or different markers.

The gene may be linked to the complex as such or protected by any suitable system normally used for transfection such as viral vectors or artificial viral envelope, liposomes or micellas, wherein the system is linked to the complex.

Numerous techniques for introducing DNA into eukaryotic cells are known to the skilled artisan. Often this is done by means of vectors, and often in the form of nucleic acid encapsidated by a (frequently virus-like) proteinaceous coat. Gene delivery systems may be applied to a wide range of clinical as well as experimental applications.

Vectors containing useful elements such as selectable and/or amplifiable markers, promoter/enhancer elements for expression in mammalian, particularly human, cells, and which may be used to prepare stocks of construct DNAs and for carrying out transfections are well known in the art. Many are commercially available.

Various techniques have been developed for modification of target tissue and cells in vivo. A number of virus vectors, discussed below, are known which allow transfection and random integration of the virus into the host. See, for example, Dubensky et al. (1984) Proc. Natl. Acad. Sci. USA 81:7529-7533; Kaneda et al., (1989) Science 243:375-378; Hiebert et al. (1989) Proc. Natl. Acad. Sci. USA 86:3594-3598; Hatzoglu et al., (1990) J. Biol. Chem. 265:17285-17293; Ferry et al. (1991) Proc. Natl. Acad. Sci. USA 88:8377-8381. Routes and modes of administering the vector include injection, e.g intravascularly or intramuscularly, inhalation, or other parenteral administration.

Advantages of adenovirus vectors for human gene therapy include the fact that recombination is rare, no human malignancies are known to be associated with such viruses, the adenovirus genome is double stranded DNA which can be manipulated to accept foreign genes of up to 7.5 kb in size, and live adenovirus is a safe human vaccine organisms.

Another vector which can express the DNA molecule of the present invention, and is useful in gene therapy, particularly in humans, is vaccinia virus, which can be rendered non-replicating (U.S. Pat. Nos. 5,225,336; 5,204,243; 5,155,020; 4,769,330).

Based on the concept of viral mimicry, artificial viral envelopes (AVE) are designed based on the structure and composition of a viral membrane, such as HIV-1 or RSV and used to deliver genes into cells in vitro and in vivo. See, for example, U.S. Pat. No. 5,252,348, Schreier H. et al., J. Mol. Recognit., 1995, 8:59-62; Schreier H et al., J. Biol. Chem., 1994, 269:9090-9098; Schreier, H., Pharm. Acta Helv. 1994, 68:145-159; Chander, R et al. Life Sci., 1992, 50:481-489, which references are hereby incorporated by reference in their entirety. The envelope is preferably produced in a two-step dialysis procedure where the “naked” envelope is formed initially, followed by unidirectional insertion of the viral surface glycoprotein of interest. This process and the physical characteristics of the resulting AVE are described in detail by Chander et al., (supra). Examples of AVE systems are (a) an AVE containing the HIV-1 surface glycoprotein gp160 (Chander et al., supra; Schreier et al., 1995, supra) or glycosyl phosphatidylinositol (GPI)-linked gp120 (Schreier et al., 1994, supra), respectively, and (b) an AVE containing the respiratory syncytial virus (RSV) attachment (G) and fusion (F) glycoproteins (Stecenko, A. A. et al., Pharm. Pharmacol. Lett. 1:127-129 (1992)). Thus, vesicles are constructed which mimic the natural membranes of enveloped viruses in their ability to bind to and deliver materials to cells bearing corresponding surface receptors.

AVEs are used to deliver genes both by intravenous injection and by instillation in the lungs. For example, AVEs are manufactured to mimic RSV, exhibiting the RSV F surface glycoprotein which provides selective entry into epithelial cells. F-AVE are loaded with a plasmid coding for the gene of interest, (or a reporter gene such as CAT not present in mammalian tissue).

The AVE system described herein in physically and chemically essentially identical to the natural virus yet is entirely “artificial”, as it is constructed from phospholipids, cholesterol, and recombinant viral surface glycoproteins. Hence, there is no carryover of viral genetic information and no danger of inadvertant viral infection. Construction of the AVEs in two independent steps allows for bulk production of the plain lipid envelopes which, in a separate second step, can then be marked with the desired viral glycoprotein, also allowing for the preparation of protein cocktail formulations if desired.

Another delivery vehicle for use in the present invention are based on the recent description of attenuated Shigella as a DNA delivery system (Sizemore, D. R. et al., Science 270:299-302 (1995), which reference is incorporated by reference in its entirety). This approach exploits the ability of Shigellae to enter epithelial cells and escape the phagocytic vacuole as a method for delivering the gene construct into the cytoplasm of the target cell. Invasion with as few as one to five bacteria can result in expression of the foreign plasmid DNA delivered by these bacteria.

A preferred type of mediator of nonviral transfection in vitro and in vivo is cationic (ammonium derivatized) lipids. These positively charged lipids form complexes with negatively charged DNA, resulting in DNA charged neutralization and compaction. The complexes endocytosed upon association with the cell membrane, and the DNA somehow escapes the endosome, gaining access to the cytoplasm. Cationic lipid:DNA complexes appear highly stable under normal conditions. Studies of the cationic lipid DOTAP suggest the complex dissociates when the inner layer of the cell membrane is destabilized and anionic lipids from the inner layer displace DNA from the cationic lipid. Several cationic lipids are available commercially. Two of these, DMRI and DC-cholesterol, have been used in human clinical trials. First generation cationic lipids are less efficient than viral vectors. For d livery to lung, any inflammatory responses accompanying the liposome administration ar reduced by changing the delivery mode to aerosol administration which distributes the dose more evenly.

Drug Screening

Genes identified as changing in various stages of colorectal cancer can be used as markers for drug screening. Thus by treating colorectal cancer cells with test compounds or extracts, and monitoring the expression of genes identified as changing in the progression of colorectal cancers, one can identify compounds or extracts which change expression of genes to a pattern which is of an earlier stage or even of normal colorectal mucosa.

The following are non-limiting examples illustrating the present invention.

Experimentals

We have used two different approaches to identify tumor suppressors, oncogenes and classifiers. The first approach was based on a spreadsheet approach in which we used the fold change and the pattern of expression being present or absent in the different preparations of RNA. The second approach was based on a mathematical approach in which we used correlation to a predefined profile as selection criteria based on Pearsons correlation coefficient.

### EXAMPLES

### Example 1

Quantification of Gene Expression Using Microarrays

Material

Colon tumor and normal oral resection edge biopsies were sampled from each patient after informed consent was obtained, and after removal of the necessary amount of tissue for routine pathological examination. Number of Tissue examined was: Normal resection edge 6, Dukes A, 5; B, 6; C, 6; D,4. The six normal tissue samples were all from Dukes A individuals.

RNA from Different tumors of the same stage were combined to form each pool. Five isuch pools were prepared as Normal pool, Dukes A pool, Dukes B pool, Dukes C pool, Dukes D pool. All tumors and normal tissue specimens were from the sigmoid or upper rectum.

Preparation of mRNA

Total mRNA was isolated using the RNAzol B RNA isolation method (WAK-Chemie Medical GMBH). Poly (A)+ RNA was isolated by an oligo-dT selection step (Oligotex mRNA kit from Qiagen).

Preparation of cRNA

One μg mRNA was used as starting material for the cDNA preparation. The first and second strand cDNA synthesis was performed using the SuperScript Choice System (Life Technologies) according to the manufacturer's instructions, except that an oligo-dT primer containing a T7 RNA polymerase promoter site was used. Labeled cRNA was prepared using the MEGAscript In Vitro Transcription kit (Ambion). Biotin labeled CTP and UTP (Enzo) was used in the reaction together with unlabeled NTP's. Following the IVT reaction, the unincorporated nucleotides were removed using RNeasy columns (Qiagen).

Array Hybridization and Scanning

Ten μg of cRNA was fragmented at 94° C. for 35 min. In a fragmentation buffer containing 40 mM Tris-acetate pH 8.1, 100 mM KOAc, 30 mM MgOAc. Prior to hybridization, the fragmented cRNA in a 6×SSPE-T hybridization buffer (1 M NaCL, 10 mM Tris pH 7.6, 0.005% Triton) was heated to 95° C. for 5 min. And subsequently to 40° C. for 5 min. Before loading onto an Affymetrix probe array cartridge. The probe array was then incubated for 16 h at 40° C. at constant rotation (60 rpm). The washing and staining procedure was performed in the Affymetrix Fluidics Station. The probe array was exposed to 10 washes in 6×SSPE-T at 25° C. followed by 4 washes in 0.5×SSPE-T at 50° C. The biotinylated cRNA was stained with a streptavidin-phycoerythrin conjugate, 10 μg/ml (Molecular Probes, Eugene, Oreg.) in 6×SSPE-T for 30 min. at 25° C. followed by 10 washes in 6×SSPE-T at 25° C. The prove arrays were scanned at 560 nm using a confocal laser scanning microscope with an argon ion laser as the excitation source (made for Affymetrix by Molecular Dynamics). Following this scan, the array was incubated with an anti-avidin antibody and an biotinylated anti-immunoglobulin, and the streptavidin-phycoerythrin step was repeated.

The readings from the quantitative scanning were analyzed by the Affymetrix Gene Expression Analysis Software.

Normalization of Data

To compare samples, normalization of the data was necessary. For that purpose we compared scaling to total GAPDH intensity (sum of 3′, middle, 5′ probe sets) of 7000 units with scaling to a total array intensity (global scaling) of 281850 units (averaging 150 units per probe set). Both gave similar results with scaling factors that differed less than ten percent in a set of experiments. Based on this we chose the global scaling for all experiments.

### Example 2

Change of Transcript Level During the Progression of Colon Cancer

Biopsies from human colon tumors were analyzed as pools of tumors representing the different stages in the progression of the colon cancer disease. A total of 4 tumor pools were used, each pool made by combining four to six tumors (see materials and methods). To generate a normal reference material, we pooled biopsies from normal colon mucosa from six volunteers.

From the biopsies RNA was extracted, reverse transcribed to cDNA and the cDNA transcribed into labelled cRNA, that was incubated on the array cartridges followed by scanning and scaling to a global array intensity amounting to 150 units per probe set. The scaling made it possible to compare individual experiments to each other. To verify the reproducibility, double determinations were made in selected cases and showed a good correlation.

The software GeneArray Analysis Suite 3.1 from Affymetrix, Inc. Was used to analyse the array data. In this software, increased levels indicat that the transcript is either up-regulated at the stated level or turned on de novo reaching a given fold above the background level. Decreased levels in a similar way indicate reduction or loss of transcript. Alterations of a single transcript during the progression of the colon cancer disease can follow several different pathways. Some of the transcript changes reflect the transition from normal cells to tumor cells, Others an increase in malignancy from Dukes A to Dukes B.

### Example 2

A. Finding Classifiers of and Predictors etc. of Colorectal Cancer Based on a Spreadsheat Approach.

We used a spreadsheat to sort genes based on different parameters obtained from the Affymetrix analysis software.

The mRNA expression analysis on the AFFYMETRIX ARRAYs resulted in 42.843 datasets identifying individual genes (table I) or EST's (table II),altogether. These were obtained from the 6.8 k Arrays (7.129 datasets) and the EST ARRAYs (35.714 datasets)

Description of the Sorting Procedure for the Spreadsheat Sorting,

Per dataset the following was listed,

Probe Set No., Present or absent in Normal tissue or the different Duke's types, gene name or homoogy or number, “AvgDiff” which is the level of expression, “Abs Call” which determines if the gene is present (P) or absent (A), “Diff call” which determines the alteration as increasing (I) or decreasing (D), “fold change” the fold change from normal tissue expression level, and the “sort score” which determines the likelihood that it is real changes (if above 0.5).

The following steps were performed,

1. exclude data if “Probe Set” is an AFFX-marker (58/array or sub-array)

2. exclude data if “Diff Call” in all 4 comparisons is “NC” (no change)

3. exclude data if “Abs Call” in all 4 comparisons is “A” (absent)

4. exclude data if three “Abs call” ar “NC” and one is “Ml or MD”

5. select data with absolute value of |sort score| arbitrarily set to >=0,5

(At this step the sorting resulted in the following number of genes sorted as being of importance, 908 Genes (12,7%) and 4155 ESTs (11,6%)

6. sort according to pattern of Abs Calls (e.g. PAAAA=lost from N to tumour Duke ABCD)

7. select data with Avg Diff of >=300 (500 for some ESTs) and/or fold change >=3 (>=5 for some ESTs)

Number of genes sorted out as being of interest after this final sorting, 130 Genes (1,8%), ≈240 ESTs (0,7%)

The following tables show the genes (Table I) and EST'+s (Table II) that were identified by this approach, analyzing the hu 6.8K FI gene array. First a list of the potential tumor suppressors, then a list of the potential oncogenes, finally a list of genes that can be used to classify the different Dukes Stages. Genes that are in bold are those that we find are of the utmost interest.

The table (Table III) that follow this section are based on the hu EST arrays Hu35k Sub A,B,C,D. These are also divided into EST's that are supposed to be expressed from tumor suppressors, and oncogenes, as well as from genes that can be used as classifiers of the different Dukes stages. The most intersting Est's are shown in bold.

TABLE I
Fold Change in comparison to normal
SUPPRESSOR CLASSIFIER

| Gen nam | Acc No | Avg Diff | Avg Diff | |
|---|---|---|---|---|
| CRC classifier genes lost PAAAA or PPAAA | | N | A | B |
| “Human chromogranin A” “mRNA,” “complete cds” | J03915 | 831 | lost | lost |
| Human adipsin/complement factor D “mRNA,” com- | M84526 | 822 | lost | lost |
| plete cds | | | | |
| Homo sapiens MLC-1V/Sb isoform gene | M24248 | 799 | lost | lost |
| Human aminopeptidase N/CD13 mRNA encoding | M22324 | 657 | lost | lost |
| aminopeptidase “N,” complete cds | | | | |
| H. sapiens MT-1I mRNA | X76717 | 650 | lost | lost |
| H. sapiens GCAP-II gene | Z70295 | 572 | lost | lost |
| Human somatostatin I gene and flanks | J00306 | 516 | lost | lost |
| Human YMP “mRNA,” complete cds | U52101 | 459 | lost | lost |
| H. sapiens mRNA for beta subunit of epithelial amiloride- | X87159 | 439 | lost | lost |
| sensitive sodium channel | | | | |
| Human K12 protein precursor “mRNA,” complete cds | U77643 | 429 | 121 | lost |
| Human sulfate transporter (DTD) “mRNA,” complete cds | U14528 | 397 | lost | lost |
| Human transcription factor hGATA-6 “mRNA,” complete | U66075 | 337 | lost | lost |
| cds. | | | | |
| H. sapiens SCAD “gene,” exon 1 and joining features | Z80345 | 326 | lost | lost |
| Human S-lac lectin L-14-II (LGALS2) gene | M87860 | 301 | lost | lost |
| Human mRNA for protein tyrosine phosphatase | D15049 | 277 | 43 | lost |
| H. sapiens mRNA for tetranectin | X64559 | 235 | lost | lost |
| Human 11 kd protein “mRNA,” complete cds | U28249 | 233 | 47 | lost |
| Human anti-mullerian hormone type II receptor precursor | U29700 | 223 | lost | lost |
| “gene,” complete cds | | | | |
| Human heparin binding protein (HBp17) “mRNA,” com- | M60047 | 218 | lost | lost |
| plete cds | | | | |
| Human ADP-ribosylation factor (hARF6) “mRNA,” com- | M57763 | 209 | lost | lost |
| plete cds | | | | |
| beta -ADD = adducin beta subunit 63 kda iso- | S81083 | 188 | lost | lost |
| form/membrane skeleton protein, beta -ADD = adducin | | | | |
| beta subunit 63 kda isoform/membrane skeleton protein | | | | |
| {alternatively spliced, exon 10 to 13 region} [human, | | | | |
| Genomic, 1851 nt, segment 3 of 3]. | | | | |
| Zinc Finger Protein Znf155 | HG4243- | 186 | lost | lost |
| | HT4513 | | | |
| Human glucagon “mRNA,” complete cds | J04040 | 182 | 25 | lost |
| H. sapiens mRNA for hair “keratin,” hHb5 | X99140 | 158 | lost | lost |
| Human tubulin-folding cofactor E “mRNA,” complete cds | U61232 | 150 | lost | lost |
| Human integrin alpha-3 chain”mRNA,” complete cds | M59911 | 126 | lost | lost |
| Human NACP gene | U46901 | 123 | lost | lost |
| H. sapiens mRNA for flavin-containing monooxygenase 5 | Z47553 | 110 | lost | lost |
| (FMO5) | | | | |
| Human mRNA for ATF-a transcription factor | X52943 | 104 | lost | lost |
| H. sapiens intestinal VIP receptor related protein mRNA | X77777 | 93 | lost | lost |

| Gene name | Acc No | Avg Diff | fold change to N | |
|---|---|---|---|---|
| Only A Classifier | | N | A | |
| Homo sapiens SKBIHs “mRNA,” complete cds. | AF015913 | 188 | Lost | |
| /gb = AF015913 /ntype = RNA | | | | |
| Mucin (Gb: M22406) | HG1067- | 501 | Lost | |
| | HT1067 | | | |
| Human platelet activating factor “acetylhydrolase,” brain | U72342 | 114 | Lost | |
| “isoform,” 45 kDa subunit (LIS1) gene | | | | |
| Homo sapiens ERK activator kinase (MEK2) mRNA | L11285 | 1470 | −5.2 | |
| Human 20-kDa myosin light chain (MLC-2) “mRNA,” | J02854 | 2047 | −4.5 | |
| complete cds | | | | |
| H. sapiens lysosomal acid phosphatase gene (EC | X15525 | 285 | −4.4 | |
| 3.1.3.2) Exon 1 (and joined CDS). | | | | |
| Human mRNA for matrix Gla protein | X53331 | 1069 | −4.2 | |
| H. sapiens mRNA for diacylglycerol kinase | X62535 | 362 | −3.5 | |
| Human h at shock protein (hsp 70) gen, compl te cds. | M11717 | 405 | −3.2 | |
| Human TRPM-2 protein gen | M63379 | 1594 | −3 | |
| Only B Classifier | | N | B | |
| Human g ne for mitochondrial acetoacetyl-CoA thiolase | D10511 | 198 | lost | |
| Human mRNA for transcription factor “AREB6,” complete | D15050 | 232 | lost | |
| cds | | | | |
| Human mRNA for KIAA0248 “gen ,” partial cds | D87435 | 374 | lost | |
| Homo sapiens (clone CC6) NADH-ubiquinone oxidore- | L04490 | 683 | lost | |
| ductase subunit “mRNA,” 3′ nd cds | | | | |
| Human phosphoglucomutase 1 (PGM1) “mRNA,” | M83088 | 1096 | lost | |
| complete cds | | | | |
| Homo sapiens guanylin “mRNA,” complete cds | M97496 | 4983 | lost | |
| “Human trans-Golgi p230” “mRNA,”“complete cds” | U41740 | 131 | lost | |
| H. sapiens mRNA for vacuolar proton “ATPase,” subunit | X71490 | 414 | lost | |
| D | | | | |
| H. sapiens mRNA for 3-hydroxy-3-methylglutaryl | X83618 | 2196 | lost | |
| coenzyme A synthase | | | | |
| Human mRNA for KIAA0018 “gene,” complete cds | D13643 | 377 | −7.7 | |
| “Mucin” “1.” “” “Epithelial,” “Alt. Splice 9” | HG371- | 3296 | −4.1 | |
| | HT26388 | | | |
| H. sapiens mRNA for L-3-hydroxyacyl-CoA dehydrogen- | X96752 | 252 | −3 | |
| ase | | | | |
| Only C Classifier | | N | C | |
| Homo sapiens colon mucosa-associated (DRA) | L02785 | 2978 | Lost | |
| “mRNA,” complete cds | | | | |
| Human Ig J chain gene | M12759 | 2193 | Lost | |
| Human selenium-binding protein (hSBP) “mRNA,” | U29091 | 1849 | Lost | |
| complete cds. /gb = U29091 /ntype = RNA | | | | |
| H. sapiens mRNA for sigma 3B protein | X99459 | 722 | Lost | |
| Human ERK1 mRNA for protein serine/threonine | X60188 | 576 | Lost | |
| kinase | | | | |
| Human mRNA for mitochondrial 3-oxoacyl-CoA “thio- | D16294 | 529 | Lost | |
| lase,” complete cds | | | | |
| “Biliary” “Glycoprotein,”“Alt. Splice” “5,”“A” | HG2850- | 489 | Lost | |
| | HT4814 | | | |
| Human AQP3 gene for aquaporine 3 (water “channel),” | AB001325 | 413 | Lost | |
| partail cds | | | | |
| Human CD14 mRNA for myelid cell-specific leucine-rich | X13334 | 413 | Lost | |
| glycoprotein | | | | |
| Human thioredoxin “mRNA,” nuclear gene encoding | U78678 | 411 | Lost | |
| mitochondrial “protein,” complete cds | | | | |
| Human mitochondrial ATPase coupling factor 6 subunit | M37104 | 373 | Lost | |
| (ATP5A) “mRNA,” complete cds | | | | |
| “Human MHC class II HLA-DP light chain” “mRNA,”“ | M57466 | 327 | Lost | |
| complete cds” | | | | |
| Human mRNA for early growth response protein 1 | X52541 | 281 | Lost | |
| (hEGR1) | | | | |
| Human mRNA for mitochondrial 3-ketoacyl-CoA thiolase | D16481 | 268 | Lost | |
| beta-subunit of trifunctional “protein,” complete cds | | | | |
| Homo sapiens laminin-related protein (LamA3) “mRNA,” | L34155 | 252 | Lost | |
| complete cds | | | | |
| H. sapiens mRNA for selenoprotein P | Z11793 | 232 | Lost | |
| Human hkf-1 “mRNA,” complete cds | D76444 | 211 | Lost | |
| Homo sapiens nuclear domain 10 protein (ndp52) | U22897 | 150 | Lost | |
| “mRNA,” complete cds | | | | |
| Human X104 “mRNA,” complete cds | L27476 | 149 | Lost | |
| H. sapiens cDNA for RFG | X77548 | 130 | Lost | |
| H. sapiens mRNA for Progression Associated Protein | Y07909 | 128 | Lost | |
| Human liver “2,4-dienoyl-CoA” reductase “mRNA,” com- | U49352 | 101 | Lost | |
| plete cds | | | | |
| Human A33 antigen precursor “mRNA,” complete | U79725 | 1650 | −6.9 | |
| cds | | | | |
| H. sapiens pS2 protein gene | X52003 | 4298 | −6 | |
| Human RASF-A PLA2 “mRNA,” complete cds | M22430 | 4983 | −5.8 | |
| Homo sapiens pstl mRNA for pancreatic secretory inhi- | Y00705 | 344 | −3.1 | |
| bitor (expressed in neoplastic tissue). | | | | |
| Human CO-029 | M35252 | 3500 | −3 | |
| Only D Classifier | | N | D | |
| Human c mplement c mp nent C3 “mRNA,” alpha | K02765 | 744 | lost | |
| and beta “subunits,” complet cds | | | | |
| H. sapi ns mRNA for adenosin “triphosphatase,” | Z69881 | 439 | lost | |
| calcium | | | | |
| Human skeletal muscle LIM-protein SLIM1 “mRNA,” | U60115 | 281 | lost | |
| complete cds | | | | |
| Human plat let-derived growth factor receptor alpha | M21574 | 187 | lost | |
| (PDGFRA) “mRNA,” complete cds | | | | |
| Human mRNA for KIAA0247 “gene,” complete cds | D87434 | 172 | lost | |
| Human mRNA for KIAA0171 “gen ,” complete cds | D79993 | 151 | lost | |
| Human Down syndrome critical regi n protein (DSCR1) | U28833 | 150 | lost | |
| “mRNA,” complete cds | | | | |
| Human Ki nuclear autoantigen “mRNA,” complete cds | U11292 | 125 | lost | |
| AB Classifier | | N | A | B |
| Homo sapiens chromosome 16 BAC clone CIT987SK- | AF001548 | 3513 | −3.6 | −4.3 |
| 815A9 complete sequence. | | | | |
| Human mRNA for ATP synthase alpha “subunit,” com- | D14710 | 3580 | −3.8 | −5.6 |
| plete cds | | | | |
| BC Classifier | | N | B | C |
| Human mRNA for IgG Fc binding “protein,” complete | D84239 | 3755 | −19.3 | −7.1 |
| cds | | | | |
| H. sapiens mRNA for carcinoembryonic “antigen,” | X98311 | 2456 | −12 | −6.5 |
| CGM2 | | | | |
| “Homo sapiens (clone lamda-hPEC-3) phosphoenol- | L05144 | 2630 | −7.6 | −14.7 |
| pyruvate carboxykinase (PCK1)” “mRNA,”“complete | | | | |
| cds” | | | | |
| Human 11-beta-hydroxysteroid dehydrogenase type 2 | U26726 | 1865 | −7.1 | −4.7 |
| “mRNA,” complete cds | | | | |
| “Human intestinal mucin (MUC2)” “mRNA,”“complete | L21998 | 7803 | −5.5 | −4.2 |
| cds” | | | | |
| Human mRNA for KIAA0106 “gene,” complete cds | D14662 | 766 | −4.7 | −3.2 |
| metallothionein | V00594 | 5417 | −4 | −6.3 |

Fold Change in comparison to normal
Oncogene CLASSSIFIER

| Gene name | Acc No | Avg Diff | Avg Diff | |
|---|---|---|---|---|
| CRC classifier genes gained APPPP or AAPPP | | A | B | |
| Homo sapiens (clones “MDP4,” MDP7) microsomal | J05257 | 1606 | 1403 | gained |
| dipeptidase (MDP) “mRNA,” complete cds | | | | |
| “Homo sapiens reg gene” “homologue,”“complete | L08010 | 1165 | 294 | gained |
| cds” | | | | |
| H. sapiens mRNA for prepro-alpha2(I) collagen | Z74616 | 1003 | 905 | gained |
| “Human S-adenosylhomocysteine hydrolase (AHCY)” | M61832 | 882 | 817 | gained |
| “mRNA,” “complete cds” | | | | |
| Transcription Factor Iiia | HG4312- | 837 | 948 | gained |
| | HT4582 | | | |
| Human gene for melanoma growth stimulatory activi- | X54489 | 731 | 330 | gained |
| ty (MGSA) | | | | |
| Human stromelysin-3 mRNA | X57766 | 643 | 1116 | gained |
| CDC25Hu2 = cdc25 + homolog “[human,” “mRNA,” 3118 | S78187 | 603 | 627 | gained |
| nt] | | | | |
| Human mRNA for cripto protein | X14253 | 532 | 293 | gained |
| Human transformation-sensitive protein (IEF SSP | M86752 | 529 | 866 | gained |
| 3521) “mRNA,” complete cds | | | | |
| Human complement component 2 (C2) gene allele b | L09708 | 515 | 625 | gained |
| H. sapiens mRNA for ITBA2 protein | X92896 | 444 | 459 | gained |
| H. sapiens encoding CLA-1 mRNA | Z22555 | 422 | 549 | gained |
| “Human fibroblast growth factor receptor 4 (FGFR4)” | L03840 | 359 | 276 | gained |
| “mRNA,” “complete cds” | | | | |
| “” “Fibronectin,” “Alt. Splice 1” | HG3044- | 354 | 261 | gained |
| | HT3742 | | | |
| tyk2 | X54667 | 336 | 352 | gain d |
| Human mRNA for B-myb gene | X13293 | 333 | 322 | gained |
| “Human phosphofructokinase (PFKM)” “mRNA,”“com- | U24183 | 296 | 426 | gain d |
| plete cds” | | | | |
| Human pre-B cell enhancing factor (PBEF) “mRNA,” | U02020 | 276 | 242 | gained |
| compl te cds | | | | |
| Human SH2-containing inositol 5-phosphatase (hSHIP) | U57650 | 254 | 315 | gained |
| “mRNA,” complete cds | | | | |
| Human Interteukin 8 (IL8) “gen ,” compl te cds | M28130 | 251 | 609 | gain d |
| “Human lamin B receptor (LBR)” “mRNA,” “complete | L25931 | 239 | 193 | gained |
| cds” | | | | |
| H. sapiens mRNA for protein tyrosine phosphatase | Z48541 | 228 | 151 | gained |
| Human mRNA for unc-18 “homologue,” complete cds | D63851 | 217 | 198 | gained |
| H. sapiens mRNA for Zn-alpha2-glycoprotein | X59766 | 215 | 156 | gained |
| | Z25521 | 215 | 127 | gained |
| “Human asparagine synthetase” “mRNA,” “complete cds” | M27396 | 212 | 195 | gained |
| Human hepatitis delta antigen interacting protein A (dipA) | U63825 | 211 | 231 | gained |
| “mRNA,” complete cds | | | | |
| Human splicesomal protein (SAP 61) “mRNA,” complete | U08815 | 157 | 201 | gained |
| cds | | | | |
| Human protein kinase C-binding protein RACK7 | U48251 | 129 | 71 | gained |
| “mRNA,” partial cds | | | | |
| Human MAC30 “mRNA,” 3′ end | L19183 | 128 | 224 | gained |
| Human thrombospondin 2 (THBS2) “mRNA,” complete | L12350 | 111 | 126 | gained |
| cds | | | | |
| “Human nicotinamide N-methyltransferase (NNMT)” | U08021 | 107 | 261 | gained |
| “mRNA,” “complete cds” | | | | |
| H. sapiens mRNA for type I interstitial collagenase | X54925 | 105 | 123 | gained |
| Human cytochrome b561 gene | U29463 | 85 | 85 | gained |
| Human H19 RNA “gene,” complete cds (spliced in | M32053 | 72 | 4498 | gained |
| silico) | | | | |
| Human collagen type XVIII alpha 1 (COL18A1) “mRNA,” | L22548 | 67 | 275 | gained |
| partial cds | | | | |
| Human clone 23733 “mRNA,” complete cds. | U79274 | absent | 162 | gained |

| Gene name | Acc No | Avg Diff | fold change to N | | | |
|---|---|---|---|---|---|---|
| Only A Classifier | | A | | | | |
| Human migration inhibitory factor-related protein 8 | M21005 | 120 | GAINED | | | |
| (MRP8) “gene,” complete cds | | | | | | |
| Human acyloxyacyl hydrolase “mRNA,” complete cds | M62840 | 130 | GAINED | | | |
| Human PEP19 (PCP4) “mRNA,” complete cds | U52969 | 174 | GAINED | | | |
| H. sapiens Humig mRNA | X72755 | 118 | GAINED | | | |
| H. sapiens PISSLRE mRNA | X78342 | 125 | GAINED | | | |
| H. sapiens mRNA for twist “protein,” partial. /gb = Y11180 | Y11180 | 121 | GAINED | | | |
| /ntype = RNA | | | | | | |
| Human mRNA for TGF-beta superfamily “protein,” com- | AB000584 | 1372 | 3.5 | | | |
| plete cds | | | | | | |
| Human mRNA for “MSS1,” complete cds | D11094 | 292 | 3.1 | | | |
| Human complement factor B “mRNA,” complete cds | L15702 | 2082 | 3.3 | | | |
| “Homo sapiens GTP-binding protein (RAB2)” “mRNA,” | M28213 | 289 | 3.1 | | | |
| “complete cds” | | | | | | |
| Human translational initiation factor 2 beta subunit (eIF-2- | M29536 | 956 | 4.1 | | | |
| beta) “mRNA,” complete cds | | | | | | |
| Human E16 “mRNA,” complete cds | M80244 | 278 | 3.8 | | | |
| IEX-1 = radiation-inducible immediate-early gene “[hu- | S81914 | 1531 | 3.6 | | | |
| man,” “placenta,” mRNA “Partial,” 1223 nt] | | | | | | |
| Human CDC16Hs “mRNA,” complete cds | U18291 | 244 | 6.1 | | | |
| Human DD96 “mRNA,” complete cds | U21049 | 625 | 3.2 | | | |
| Human (memc) “mRNA,” 3′UTR. /gb = U30999 | U30999 | 256 | 3.8 | | | |
| /ntype = RNA | | | | | | |
| “Human ubiquitin-conjugating enzyme (UBE2I)” | U45328 | 448 | 10.6 | | | |
| “mRNA,” “complete cds” | | | | | | |
| “Human fetal brain glycogen phosphorylase B” “mRNA,” | U47025 | 2349 | 3.7 | | | |
| “complete cds” | | | | | | |
| “Human BTG2 (BTG2)” “mRNA,” “complete cds” | U72649 | 527 | 5.2 | | | |
| Human jun-B mRNA for JUN-B protein | X51345 | 1350 | 4.6 | | | |
| Only B Classifier | | B | | | | |
| Human adipocyte lipid-binding “protein,” complete cds | J02874 | 268 | GAINED | | | |
| Human A1 protein “mRNA,” compl te cds | U29680 | 102 | GAINED | | | |
| Human LGN protein “mRNA,” complete cds | U54999 | 110 | GAINED | | | |
| Human sk letal muscle LIM-protein SLIM2 “mRNA,” | U60116 | 109 | GAINED | | | |
| partial cds | | | | | | |
| Human mRNA for alpha1-acid glycoprotein (orosomu- | X02544 | 156 | GAINED | | | |
| coid) | | | | | | |
| Human mRNA for fibronectin receptor alpha subunit | X06256 | 46 | GAINED | | | |
| H. sapiens P1-Cdc21 mRNA | X74794 | 278 | GAINED | | | |
| H. sapiens mRNA for fibulin-2 | X82494 | 284 | GAINED | | | |
| H. sapiens 5T4 gene for 5T4 Oncofetal antigen | Z29083 | 152 | GAINED | | | |
| Homo sapiens mRNA for osteoblast specific factor 2 | D13666 | 324 | 7.6 | | | |
| (OSF-2os) | | | | | | |
| Mac25 | HG987-HT987 | 2772 | 3.3 | | | |
| “Human lysozyme” “mRNA,” “complete cds with an Alu | J03801 | 920 | 3.7 | | | |
| repeat in the 3′ flank” | | | | | | |
| Human metalloproteinase (HME) “mRNA,” complete cds | L23808 | 794 | 7.4 | | | |
| Human alpha-1 collagen type IV gene, exon 52. | M26576 | 610 | 4.9 | | | |
| Human lumican “mRNA,” complete cds | U21128 | 1105 | 4.1 | | | |
| Human mRNA for fibronectin (FN precursor) | X02761 | 4181 | 5.5 | | | |
| Human mRNA fragment for elongation factor TU (N- | X03689 | 3515 | 3.1 | | | |
| terminus). /gb = X03689 /ntype = RNA | | | | | | |
| Human mRNA for type IV collagen alpha-2 chain | X05610 | 1531 | 3 | | | |
| Human mRNA for collagen VI alpha-1 C-terminal globu- | X15880 | 2062 | 3.5 | | | |
| lar domain | | | | | | |
| “H. sapiens,” gene for Membrane cofactor protein | X59405 | 272 | 3.4 | | | |
| H. sapiens SOD-2 gene for manganese superoxide dis- | X65965 | 234 | 3.1 | | | |
| mutase. /gb = X65965 /ntype = DNA /annot = exon | | | | | | |
| H. sapiens NMB mRNA | X76534 | 338 | 3.3 | | | |
| H. sapiens vimentin gene | Z19554 | 3472 | 3.2 | | | |
| Only C Classifier | | C | | | | |
| Ribosomal Protein L39 Homolog | HG2874- | 102 | GAINED | | | |
| | HT3018 | | | | | |
| Homo sapiens (clone d2-115) kappa opioid receptor | L37362 | 168 | GAINED | | | |
| (OPRK1) “mRNA,” complete cds | | | | | | |
| Human kell blood group protein mRNA | M64934 | 143 | GAINED | | | |
| | U73167 | 374 | GAINED | | | |
| Human cancellous bone osteoblast mRNA for serin | D87258 | 504 | 3.4 | | | |
| protease with IGF-binding “motif,” complete cds | | | | | | |
| Human interferon-inducible protein 27-Sep “mRNA,” | J04164 | 7717 | 3.8 | | | |
| complete cds | | | | | | |
| “Human sickle cell beta-globin” “mRNA,” “complete cds” | M25079 | 3090 | 4.6 | | | |
| | M29277 | 1588 | 3.7 | | | |
| “Human spermidine synthase” “mRNA,” “complete cds” | M34338 | 866 | 4.1 | | | |
| Human copine 1 “mRNA,” complete cds | U83246 | 2079 | 3.7 | | | |
| Only D Classifier | | D | | | | |
| Homo sapiens FRG1 “mRNA,” complete cds | L76159 | 73 | GAINED | | | |
| Human cyclin protein “gene,” complete cds | M15796 | 149 | GAINED | | | |
| Human U2 small nuclear RNA-associated B″ antigen | M15841 | 194 | GAINED | | | |
| “mRNA,” complete cds | | | | | | |
| Human mRNA export protein Rae1 (RAE1) “mRNA,” | U84720 | 193 | GAINED | | | |
| complete cds. | | | | | | |
| Human protease-activated receptor 3 (PAR3) “mRNA,” | U92971 | 142 | GAINED | | | |
| complete cds. | | | | | | |
| H. sapiens mRNA for mediator of receptor-induced toxi- | X84709 | 200 | GAINED | | | |
| city | | | | | | |
| H. sapiens RFXAP mRNA | Y12812 | 230 | GAINED | | | |
| Human mRNA for “Qip1,” complete cds | AB002533 | 8881 | 2.7 | | | |
| Human mRNA for transferrin receptor | X01060 | 557 | 3 | | | |
| “metastasis-associated gene” “[human,” “highly metasta- | S79219 | 216 | 4 | | | |
| tic lung cell subline” “Anip[937],” “mRNA” “Partial,”“978 | | | | | | |
| nt]” | | | | | | |
| AB Classifier | | N | A | B | | |
| Human chaperonin 10 “mRNA,” complete cds | U07550 | 50 | 4.1 | 3.3 | | |
| H. sapiens RING4 cDNA | X57522 | 73 | 4.9 | 5.4 | | |
| H. sapiens genes TAP1, TAP2. LMP2, LMP7 and DOB. | X66401 | 134 | 3.2 | 3.1 | | |
| H. sapiens mRNA for alpha 4 prot in | Y08915 | 96 | 3.7 | 3.6 | | |
| Homo sapiens interl ukin-1 receptor-associat d kinase | L76191 | 285 | 3.1 | 3.1 | | |
| (IRAK) “mRNA,” complet cds | | | | | | |
| “Human von Will brand factor” “mRNA,” “3′ end” | M10321 | 84 | 3.7 | 4.1 | | |
| Human chromosome s gregation gen homolog CAS | U33286 | 86 | 4.8 | 3.6 | | |
| “mRNA,” compl te cds | | | | | | |
| Human Bruton's tyrosine kinase-associated protein-135 | U77948 | 68 | 3.4 | 4.9 | | |
| “mRNA,” complete cds. | | | | | | |
| “Human KH type splicing regulatory protein KSRP” | U94832 | 52 | 3.2 | 3.2 | | |
| “mRNA,” “complete cds.” | | | | | | |
| H. sapiens ADE2H1 mRNA showing homologies to SAI- | X53793 | 40 | 3 | 3.1 | | |
| CAR synthetase and AIR carboxylase of the purine | | | | | | |
| pathway (EC “6.3.2.6,” EC 4.1.1.21) | | | | | | |
| BC Classifier | | N | B | C | | |
| “” “Globin,” “Beta” | HG1428- | 504 | 3.1 | 4.3 | | |
| | HT1428 | | | | | |
| “Human alpha-1 collagen type 1” “gene,” “3′ end” | M55998 | 2706 | 3.1 | 3.7 | | |
| H. sapiens mRNA for SOX-4 protein | X70683 | 130 | 4.5 | 4.5 | | |
| “Human mRNA for collagen binding protein” “2,” “com- | D83174 | 131 | 8.1 | 6.1 | | |
| plete cds” | | | | | | |
| Human SPARC/osteonectin “mRNA,” complete cds | J03040 | 358 | 6.1 | 3.9 | | |
| Human PRAD1 mRNA for cyclin | X59798 | 263 | 3.3 | 3.4 | | |
| ABC Classifier | | N | A | B | C | |
| Human transforming growth factor-beta induced gene | M77349 | 426 | 4.7 | 6.7 | 4.4 | |
| product (BIGH3) “mRNA,” complete cds | | | | | | |
| “Human breast epithelial antigen BA46” “mRNA,” “com- | U58516 | 169 | 3.3 | 3.2 | 4.2 | |
| plete cds” | | | | | | |
| | X57351 | 460 | 4.8 | 3.5 | 3.7 | |
| H. sapiens NGAL gene | X99133 | 327 | 8.3 | 3.1 | 4.8 | |
| Human mRNA for MDNCF (monocyte-derived neu- | Y00787 | 87 | 5 | 9.2 | 13.4 | |
| trophil chemotactic factor) | | | | | | |
| H. sapiens EF-1 delta gene encoding human elongation | Z21507 | 198 | 4.4 | 6.8 | 4.5 | |
| factor-1-delta | | | | | | |
| H. sapiens mRNA for prepro-alpha1(I) collagen | Z74615 | 285 | 5 | 8.2 | 6.1 | |
| Nuclear Factor Nf-II6 | HG3494- | 246 | 4.3 | 4.4 | 4.2 | |
| | HT3688 | | | | | |
| | U29175 | 62 | 4.3 | 3.6 | 4.4 | |
| ABCD Classifier | | N | A | B | C | D |
| “HNL = neutrophil lipocalin” “[human,” “ovarian can- | S75256 | 361 | 8.8 | 4.3 | 7.7 | 9 |
| cer cell line” “OC6,” “mRNA” “Partial,” “534 nt]. | | | | | | |
| /gb = S75256 /ntype = RNA” | | | | | | |

TABLE II

| | | | | | | | | | | |
|---|---|---|---|---|---|---|---|---|---|---|
| | | unknown genes, data | | | | | | Diff | Fold | Sort |
| | Tumor | found after blast | | Avg | Abs | Avg | Abs | Call | Change | Score |
| EST candidates | stages | search | | Diff | Call | Diff | Call | Avs | Avs | Avs |
| Probe Set | NABCD | homologous to | EST name | N | N | A | A | N | N | N |

LOST from N to tumour (tumour suppressor)

| | | | | | | | | | | |
|---|---|---|---|---|---|---|---|---|---|---|
| RC_H04768_at | PAAAA | chrom 15 no homology | yj51f03.s1 | 1078 | P | 127 | A | D | −8.4 | −7.99 |
| | | | Homo | | | | | | | |
| | | | sapiens | | | | | | | |
| | | | cDNA | | | | | | | |
| | | | clone | | | | | | | |
| | | | 152285 3′. | | | | | | | |
| RC_Z39652_at | PAAAA | Y14593 APM-1 gene | H. sapiens | 959 | P | −157 | A | D | ˜−19.7 | −15.28 |
| | | adipocyte-specific | partial | | | | | | | |
| | | secretory protein: | cDNA | | | | | | | |
| | | chrom 1q21.3-q23 | sequence; | | | | | | | |
| | | | clone | | | | | | | |
| | | | c1fg03. | | | | | | | |
| RC_H30270_at | PAAAA | chrom 18 PAAAA in | yp42e02.s1 | 941 | P | 366 | A | D | −4.1 | −3.11 |
| | | colon & bladder no | Homo | | | | | | | |
| | | homology | sapiens | | | | | | | |
| | | | cDNA | | | | | | | |
| | | | clone | | | | | | | |
| | | | 190106 3′ | | | | | | | |
| | | | similar to | | | | | | | |
| | | | contains | | | | | | | |
| | | | MSR1 | | | | | | | |
| | | | repetitive | | | | | | | |
| | | | element;. | | | | | | | |
| RC_T47089_s_at | PAAAA | tenascin-X; tenascin- | yb52b08.s1 | 815 | P | −49 | A | D | ˜−31.4 | −18.16 |
| | | X precursor; unidenti- | Homo | | | | | | | |
| | | fied protein | sapiens | | | | | | | |
| | | | cDNA | | | | | | | |
| | | | clone | | | | | | | |
| | | | 74775 3′ | | | | | | | |
| | | | similar to | | | | | | | |
| | | | gb: M25813 | | | | | | | |
| | | | FIBRI- | | | | | | | |
| | | | NOGEN- | | | | | | | |
| | | | LIKE | | | | | | | |
| | | | PROTEIN | | | | | | | |
| | | | (HUMAN). | | | | | | | |
| RC_W31906_at | PAAAA | secretagogin; | zc76c03.s1 | 736 | P | 243 | A | D | −4.8 | −3.12 |
| | | dJ501N12.8 (putative | Pancre- | | | | | | | |
| | | protein) chrom 6 | atic Islet | | | | | | | |
| | | | Homo | | | | | | | |
| | | | sapiens | | | | | | | |
| | | | cDNA | | | | | | | |
| | | | clone | | | | | | | |
| | | | 328228 3′. | | | | | | | |
| RC_AA279803_at | PAAAA | chrom 2 no homology | zs92a11.s1 | 643 | P | 164 | A | D | −3.9 | −2.35 |
| | | | NCI_CGAP_{—} | | | | | | | |
| | | | GCB1 | | | | | | | |
| | | | Homo | | | | | | | |
| | | | sapiens | | | | | | | |
| | | | cDNA | | | | | | | |
| | | | clone | | | | | | | |
| | | | IMAGE: | | | | | | | |
| | | | 704924 3′. | | | | | | | |
| RC_R01646_at | PAAAA | chrom 13q32.1-33.3; | ye79f11.s1 | 607 | P | −75 | A | D | ˜−17.7 | −13.83 |
| | | AL159152; homolo- | Homo | | | | | | | |
| | | gy to mouse Pcbp1 - | sapiens | | | | | | | |
| | | poly(rC)-binding | cDNA | | | | | | | |
| | | protein 1 | clone | | | | | | | |
| | | | 123981 3′. | | | | | | | |
| RC_AA099820_at | PAAAA | BAC clone AC016778 | zk87c05.s1 | 587 | P | 58 | A | D | ˜−9.5 | −6.53 |
| | | | Soares | | | | | | | |
| | | | pregnant | | | | | | | |
| | | | uterus | | | | | | | |
| | | | NbHPU | | | | | | | |
| | | | Homo | | | | | | | |
| | | | sapiens | | | | | | | |
| | | | cDNA | | | | | | | |
| | | | clone | | | | | | | |
| | | | 489800 3′. | | | | | | | |
| AA319615_at | PAAAA | secretory carrier | EST21862 | 568 | P | −100 | A | D | ˜−34.7 | −15.76 |
| | | membrane protein; | Adrenal | | | | | | | |
| | | secretory carrier | gland | | | | | | | |
| | | membrane protein 2; | tumor | | | | | | | |
| | | chrom 15 | Homo | | | | | | | |
| | | | sapiens | | | | | | | |
| | | | cDNA 5′ | | | | | | | |
| | | | end. | | | | | | | |
| H07011_at | PAAAA | tetraspan NET-6 | yl81e01.r1 | 324 | P | 123 | A | D | −2.6 | −0.78 |
| | | mRNA; transmem- | Homo | | | | | | | |
| | | brane 4 superfamlly; | sapiens | | | | | | | |
| | | chrom 7 | cDNA | | | | | | | |
| | | | clone | | | | | | | |
| | | | 44466 5′. | | | | | | | |
| RC_T68873_f_at | PPAAA | | yc30f03.s1 | 3837 | P | 611 | P | D | −6 | −10.26 |
| | | | Homo | | | | | | | |
| | | | sapiens | | | | | | | |
| | | | cDNA | | | | | | | |
| | | | clone | | | | | | | |
| | | | 82205 3′ | | | | | | | |
| | | | similar to | | | | | | | |
| | | | gb: J00272_{—} | | | | | | | |
| | | | rna1 | | | | | | | |
| | | | Human | | | | | | | |
| | | | metallothi- | | | | | | | |
| | | | onein-II | | | | | | | |
| | | | pseudo- | | | | | | | |
| | | | gene (HU- | | | | | | | |
| | | | MAN); con- | | | | | | | |
| | | | tains L1 | | | | | | | |
| | | | repetitive | | | | | | | |
| | | | element;. | | | | | | | |
| RC_T40995_f_at | PPAAA | | ya15e08.s3 | 1973 | P | 841 | P | D | −2.7 | −2.24 |
| | | | Homo | | | | | | | |
| | | | sapiens | | | | | | | |
| | | | cDNA | | | | | | | |
| | | | clone | | | | | | | |
| | | | 61574 3′. | | | | | | | |
| RC_H81070_f_at | PPAAA | | yu60h05.s1 | 1683 | P | 469 | P | D | −4.3 | −4.92 |
| | | | Homo | | | | | | | |
| | | | sapiens | | | | | | | |
| | | | cDNA | | | | | | | |
| | | | clone | | | | | | | |
| | | | 230553 3′ | | | | | | | |
| | | | similar to | | | | | | | |
| | | | gb: X64177 | | | | | | | |
| | | | H. sapiens | | | | | | | |
| | | | mRNA for | | | | | | | |
| | | | metallothi- | | | | | | | |
| | | | onein | | | | | | | |
| | | | (HUMAN);. | | | | | | | |
| RC_N30796_at | PPPAA | | yw65d03.s1 | 1338 | P | 584 | P | D | −2.7 | −1.48 |
| | | | Homo | | | | | | | |
| | | | sapiens | | | | | | | |
| | | | cDNA | | | | | | | |
| | | | clone | | | | | | | |
| | | | 257093 3′. | | | | | | | |
| RC_W37778_f_at | PPAAA | | zc13b12.s1 | 945 | P | 371 | P | D | −3.5 | −2.53 |
| | | | Soares | | | | | | | |
| | | | parathy- | | | | | | | |
| | | | roid tumor | | | | | | | |
| | | | NbHPA | | | | | | | |
| | | | Homo | | | | | | | |
| | | | sapiens | | | | | | | |
| | | | cDNA | | | | | | | |
| | | | clone | | | | | | | |
| | | | 322175 3′ | | | | | | | |
| | | | similar to | | | | | | | |
| | | | contains | | | | | | | |
| | | | LTR2.t3 | | | | | | | |
| | | | LTR2 | | | | | | | |
| | | | repetitive | | | | | | | |
| | | | element;. | | | | | | | |
| RC_R70212_s_at | PPAAA | | yj80d09.s1 | 718 | P | 227 | P | D | −3.1 | −1.57 |
| | | | Homo | | | | | | | |
| | | | sapiens | | | | | | | |
| | | | cDNA | | | | | | | |
| | | | clone | | | | | | | |
| | | | 155057 3′ | | | | | | | |
| | | | similar to | | | | | | | |
| | | | gb: U05259_{—} | | | | | | | |
| | | | rna1 MB- | | | | | | | |
| | | | 1 MEM- | | | | | | | |
| | | | BRANE | | | | | | | |
| | | | GLY- | | | | | | | |
| | | | COPRO- | | | | | | | |
| | | | TEIN | | | | | | | |
| | | | PRECUR- | | | | | | | |
| | | | SOR | | | | | | | |
| | | | (HUMAN);. | | | | | | | |
| RC_AA426330_at | PPAAA | | zw11h09.s1 | 676 | P | 164 | P | D | −4.1 | −2.75 |
| | | | Soares | | | | | | | |
| | | | NhHMPu | | | | | | | |
| | | | S1 Homo | | | | | | | |
| | | | sapiens | | | | | | | |
| | | | cDNA | | | | | | | |
| | | | clone | | | | | | | |
| | | | 769025 3′. | | | | | | | |
| RC_N33927_s_at | PPAAA | | yv25e09.s1 | 599 | P | 312 | P | D | −19 | −0.46 |
| | | | Homo | | | | | | | |
| | | | sapiens | | | | | | | |
| | | | cDNA | | | | | | | |
| | | | clone | | | | | | | |
| | | | 243784 3′. | | | | | | | |
| RC_T90190_s_at | PPAAA | | yd38f12.s1 | 574 | P | 102 | P | D | −4.8 | −2.79 |
| | | | Homo | | | | | | | |
| | | | sapiens | | | | | | | |
| | | | cDNA | | | | | | | |
| | | | clone | | | | | | | |
| | | | 110543 3′. | | | | | | | |
| RC_AA447145_at | PPPAA | | zw93f08.s1 | 374 | P | 123 | P | D | −3 | −1.14 |
| | | | Soares | | | | | | | |
| | | | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 784551 3′. | | | | | | | |
| RC_H75860_at | PPAAA | | yu60g09.s1 | 355 | P | 70 | P | D | −4 | −1.64 |
| | | | Homo | | | | | | | |
| | | | sapiens | | | | | | | |
| | | | cDNA | | | | | | | |
| | | | clone | | | | | | | |
| | | | 230560 3′. | | | | | | | |
| RC_T71132_s_at | PPAAA | | yd34a02.s1 | 325 | P | 131 | P | D | −2.6 | −0.81 |
| | | | Homo | | | | | | | |
| | | | sapiens | | | | | | | |
| | | | cDNA | | | | | | | |
| | | | clone | | | | | | | |
| | | | 110090 3′ | | | | | | | |
| | | | similar to | | | | | | | |
| | | | gb: L13288 | | | | | | | |
| | | | VASOAC- | | | | | | | |
| | | | TIVE | | | | | | | |
| | | | INTESTI- | | | | | | | |
| | | | NAL POL- | | | | | | | |
| | | | YPEPTIDE | | | | | | | |
| | | | RECEPTOR | | | | | | | |
| | | | 1 PRE- | | | | | | | |
| | | | CURSOR | | | | | | | |
| | | | (HUMAN);. | | | | | | | |

GAINED from N to tumour
(oncogene)

| | | | | | | | | | | |
|---|---|---|---|---|---|---|---|---|---|---|
| RC_AA609013_s_{—} | APPPP | microsomal dipepti- | af05f12.s1 | 421 | A | 2176 | P | I | 5.1 | 6.42 |
| at | | dase (also on 6.8 k); | Soares | | | | | | | |
| | | chrom 16 | testis NHT | | | | | | | |
| | | | Homo | | | | | | | |
| | | | sapiens | | | | | | | |
| | | | cDNA | | | | | | | |
| | | | clone | | | | | | | |
| | | | 1030799 3′ | | | | | | | |
| | | | similar to | | | | | | | |
| | | | gb: J05257 | | | | | | | |
| | | | MICRO- | | | | | | | |
| | | | SOMAL | | | | | | | |
| | | | DIPEPTI- | | | | | | | |
| | | | DASE | | | | | | | |
| | | | PRECUR- | | | | | | | |
| | | | SOR | | | | | | | |
| | | | (HUMAN);. | | | | | | | |
| RC_AA232508_at | APPPP | CGI-89 protein; un- | zr28d08.s1 | 516 | A | 2511 | P | I | 4.5 | 5.56 |
| | | named protein product; | Strata- | | | | | | | |
| | | hypothetical protein | gene NT2 | | | | | | | |
| | | | neuronal | | | | | | | |
| | | | precursor | | | | | | | |
| | | | 937230 | | | | | | | |
| | | | Homo | | | | | | | |
| | | | sapiens | | | | | | | |
| | | | cDNA | | | | | | | |
| | | | clone | | | | | | | |
| | | | 664719 3′. | | | | | | | |
| RC_AA428964_at | APPPP | serine protease-like | zw19b02.s1 | 395 | A | 559 | P | I | 1.4 | 0.13 |
| | | protease; serine pro- | Soares | | | | | | | |
| | | tease homolog = NES1; | ovary | | | | | | | |
| | | normal epithelial cell- | tumor | | | | | | | |
| | | specific 1 | NbHOT | | | | | | | |
| | | | Homo | | | | | | | |
| | | | sapiens | | | | | | | |
| | | | cDNA | | | | | | | |
| | | | clone | | | | | | | |
| | | | 769707 3′ | | | | | | | |
| | | | similar to | | | | | | | |
| | | | gb: X57025_{—} | | | | | | | |
| | | | rna1 | | | | | | | |
| | | | INSULIN- | | | | | | | |
| | | | LIKE | | | | | | | |
| | | | GROWTH | | | | | | | |
| | | | FACTOR | | | | | | | |
| | | | IA PRE- | | | | | | | |
| | | | CURSOR | | | | | | | |
| | | | (HUMAN);. | | | | | | | |
| RC_T52813_s_at | APPPP | dJ28O10.2 (G0S2 | ya79h10.s1 | 191 | A | 471 | P | I | 3.1 | 1.2 |
| | | (PUTATIVE LYM- | Homo | | | | | | | |
| | | PHOCYTE G0/G1 | sapiens | | | | | | | |
| | | SWITCH PROTEIN 2; | cDNA | | | | | | | |
| | | chrom 1 | clone | | | | | | | |
| | | | 67939 3′ | | | | | | | |
| | | | similar to | | | | | | | |
| | | | gb: M69199 | | | | | | | |
| | | | PUTA- | | | | | | | |
| | | | TIVE LYM- | | | | | | | |
| | | | PHOCYTE | | | | | | | |
| | | | G0/G1 | | | | | | | |
| | | | SWITCH | | | | | | | |
| | | | PROTEIN | | | | | | | |
| | | | (HUMAN). | | | | | | | |
| RC_AA075642_at | APPPP | gp-340 variant protein; | zm88a11.s1 | 264 | A | 3364 | P | I | 13 | 20.97 |
| | | DMBT1/8kb.2 protein | Strata- | | | | | | | |
| | | | gene | | | | | | | |
| | | | ovarian | | | | | | | |
| | | | cancer | | | | | | | |
| | | | (#937219) | | | | | | | |
| | | | Homo | | | | | | | |
| | | | sapiens | | | | | | | |
| | | | cDNA | | | | | | | |
| | | | clone | | | | | | | |
| | | | 544988 3′. | | | | | | | |
| RC_AA007218_at | APPPP | chrom 13 no homology | 13cDNA54- | 156 | A | 902 | P | I | 5.8 | 4.88 |
| | | | 3.seq | | | | | | | |
| | | | Soares | | | | | | | |
| | | | infant | | | | | | | |
| | | | brain 1NIB | | | | | | | |
| | | | Homo | | | | | | | |
| | | | sapiens | | | | | | | |
| | | | cDNA | | | | | | | |
| | | | clone | | | | | | | |
| | | | HY18-131 | | | | | | | |
| | | | 3′. | | | | | | | |
| RC_N33920_at | APPPP | ubiquitin-like protein | yv25b11.s1 | 15 | A | 1173 | P | I | 34.5 | 22.41 |
| | | FAT10; diubiquitin; | Homo | | | | | | | |
| | | dJ271M21.6 (Diubiqui- | sapiens | | | | | | | |
| | | tin); chrom 6 | cDNA | | | | | | | |
| | | | clone | | | | | | | |
| | | | 243741 3′. | | | | | | | |
| RC_N71781_at | APPPP | KIAA1199 protein, | yz94e06.s1 | 9 | A | 575 | P | I | ˜16.7 | 10.24 |
| | | chrom 15 | Homo | | | | | | | |
| | | | sapiens | | | | | | | |
| | | | cDNA | | | | | | | |
| | | | clone | | | | | | | |
| | | | 290722 3′. | | | | | | | |
| RC_R67275_s_at | APPPP | alpha-1 (type XI) | yh01f11.s2 | 17 | A | 403 | P | I | ˜10.9 | 6.01 |
| | | collagen precursor; | Homo | | | | | | | |
| | | collagen, type XI, | sapiens | | | | | | | |
| | | alpha 1; collagen type | cDNA | | | | | | | |
| | | XI alpha-1 isoform A; | clone | | | | | | | |
| | | chrom 1 | 41676 3′ | | | | | | | |
| | | | similar to | | | | | | | |
| | | | gb: J04177 | | | | | | | |
| | | | COLLA- | | | | | | | |
| | | | GEN | | | | | | | |
| | | | ALPHA | | | | | | | |
| | | | 1(XI) | | | | | | | |
| | | | CHAIN | | | | | | | |
| | | | PRECUR- | | | | | | | |
| | | | SOR | | | | | | | |
| | | | (HUMAN);. | | | | | | | |
| RC_W80763_at | APPPP | hypothetical protein; | zd83g04.s1 | 193 | A | 571 | P | I | 3.6 | 1.97 |
| | | chrom 17 | Soares | | | | | | | |
| | | | fetal heart | | | | | | | |
| | | | NbHH19W | | | | | | | |
| | | | Homo | | | | | | | |
| | | | sapiens | | | | | | | |
| | | | cDNA | | | | | | | |
| | | | clone | | | | | | | |
| | | | 347286 3′. | | | | | | | |
| RC_AA443793_at | APPPP | chrom 7p22 | zw86e11.s1 | 232 | A | 781 | P | I | 3.5 | 2.19 |
| | | AC006028 BAC clone | Soares | | | | | | | |
| | | | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 783884 3′. | | | | | | | |
| RC_AA034499_s_{—} | APPPP | ZNF198 protein; zinc | zk23c04.s1 | 128 | A | 589 | P | I | 4.7 | 2.97 |
| at | | finger protein; FIM | Soares | | | | | | | |
| | | protein; Cys-rich pro- | pregnant | | | | | | | |
| | | tein; zinc finger protein | uterus | | | | | | | |
| | | 198; chrom 13 | NbHPU | | | | | | | |
| | | | Homo | | | | | | | |
| | | | sapiens | | | | | | | |
| | | | cDNA | | | | | | | |
| | | | clone | | | | | | | |
| | | | 471366 3′. | | | | | | | |
| RC_AA035482_at | APPPP | chrom 5; AK022505 | zk27b07.s1 | 260 | A | 562 | P | I | 1.7 | 0.3 |
| | | clone; CalcineurinB | Soares | | | | | | | |
| | | (weakly similar) | pregnant | | | | | | | |
| | | | uterus | | | | | | | |
| | | | NbHPU | | | | | | | |
| | | | Homo | | | | | | | |
| | | | sapiens | | | | | | | |
| | | | cDNA | | | | | | | |
| | | | clone | | | | | | | |
| | | | 471733 3′. | | | | | | | |
| RC_AA024482_at | APPPP | hypothetical protein; | ze76a01.s1 | −28 | A | 828 | P | I | ˜44.0 | 19.93 |
| | | unnamed protein | Soares | | | | | | | |
| | | product; chrom 17 | fetal heart | | | | | | | |
| | | | NbHH19W | | | | | | | |
| | | | Homo | | | | | | | |
| | | | sapiens | | | | | | | |
| | | | cDNA | | | | | | | |
| | | | clone | | | | | | | |
| | | | 364872 3′. | | | | | | | |
| RC_H93021_at | APPPP | chrom 2; XM_004890 | yv06a03.s1 | 274 | A | 345 | P | I | 3 | 1.29 |
| | | peptidylprolyl isomer- | Homo | | | | | | | |
| | | ase A (cyclophilin A) | sapiens | | | | | | | |
| | | | cDNA | | | | | | | |
| | | | clone | | | | | | | |
| | | | 241900 3′ | | | | | | | |
| | | | similar to | | | | | | | |
| | | | gb: X52851_{—} | | | | | | | |
| | | | rna1 PEP- | | | | | | | |
| | | | TIDYL- | | | | | | | |
| | | | PROLYL | | | | | | | |
| | | | CIS- | | | | | | | |
| | | | TRANS | | | | | | | |
| | | | ISOMER- | | | | | | | |
| | | | ASE A | | | | | | | |
| | | | (HUMAN);. | | | | | | | |
| RC_AA427737_at | APPPP | no homology | zw30g12.s1 | 145 | A | 339 | P | I | 2.3 | 0.6 |
| | | | Soares | | | | | | | |
| | | | ovary | | | | | | | |
| | | | tumor | | | | | | | |
| | | | NbHOT | | | | | | | |
| | | | Homo | | | | | | | |
| | | | sapiens | | | | | | | |
| | | | cDNA | | | | | | | |
| | | | clone | | | | | | | |
| | | | 770854 3′. | | | | | | | |
| RC_AA417078_at | APPPP | chrom 7q31; | zu13c02.s1 | 59 | A | 405 | P | I | 3.2 | 1.21 |
| | | AF017104 clone | Soares | | | | | | | |
| | | | testis NHT | | | | | | | |
| | | | Homo | | | | | | | |
| | | | sapiens | | | | | | | |
| | | | cDNA | | | | | | | |
| | | | clone | | | | | | | |
| | | | 731714 3′. | | | | | | | |
| M29873_s_at | APPPP | cytochrome P450-IIB | Human | 19 | A | 640 | P | I | ˜32.3 | 15.15 |
| | | (hIIB3); 19q13.1- | cytochrome | | | | | | | |
| | | q13.2 | P450- | | | | | | | |
| | | | IIB (hIIB3) | | | | | | | |
| | | | mRNA, | | | | | | | |
| | | | complete | | | | | | | |
| | | | cds. | | | | | | | |
| RC_H27498_f_at | AAPPP | | yl57a08.s1 | 2557 | A | 1129 | A | NC | −2.3 | −1.52 |
| | | | Homo | | | | | | | |
| | | | sapiens | | | | | | | |
| | | | cDNA | | | | | | | |
| | | | clone | | | | | | | |
| | | | 162326 3′ | | | | | | | |
| | | | similar to | | | | | | | |
| | | | gb: S55735 | | | | | | | |
| | | | IG AL- | | | | | | | |
| | | | PHA-1 | | | | | | | |
| | | | CHAIN C | | | | | | | |
| | | | REGION | | | | | | | |
| | | | (HUMAN);. | | | | | | | |
| RC_T92363_s_at | AAPPP | | ye19h06.s1 | 68 | A | 255 | A | NC | 3.7 | 1.37 |
| | | | Homo | | | | | | | |
| | | | sapiens | | | | | | | |
| | | | cDNA | | | | | | | |
| | | | clone | | | | | | | |
| | | | 118235 3′. | | | | | | | |
| RC_N89910_at | AAAPP | | zb22f11.s1 | 205 | A | 226 | A | NC | 1.1 | 0.01 |
| | | | Soares | | | | | | | |
| | | | fetal lung | | | | | | | |
| | | | NbHL 19W | | | | | | | |
| | | | Homo | | | | | | | |
| | | | sapiens | | | | | | | |
| | | | cDNA | | | | | | | |
| | | | clone | | | | | | | |
| | | | 302829 3′ | | | | | | | |
| | | | similar to | | | | | | | |
| | | | SW: PGT_{—} | | | | | | | |
| | | | RAT | | | | | | | |
| | | | Q00910 | | | | | | | |
| | | | PROSTAG | | | | | | | |
| | | | LANDIN | | | | | | | |
| | | | TRANS- | | | | | | | |
| | | | PORTER;. | | | | | | | |
| RC_W60516_at | AAAPP | | zc99f02.s1 | 391 | A | 285 | A | NC | −1.1 | −0.02 |
| | | | Pancreatic | | | | | | | |
| | | | Islet Homo | | | | | | | |
| | | | sapiens | | | | | | | |
| | | | cDNA | | | | | | | |
| | | | clone | | | | | | | |
| | | | 339291 3′ | | | | | | | |
| | | | similar to | | | | | | | |
| | | | contains | | | | | | | |
| | | | Alu repeti- | | | | | | | |
| | | | tive ele- | | | | | | | |
| | | | ment;. | | | | | | | |
| RC_AA219699_at | AAAPP | | zr03d01.s1 | 15 | A | 110 | A | NC | ˜2.6 | 0.37 |
| | | | Strata- | | | | | | | |
| | | | gene NT2 | | | | | | | |
| | | | neuronal | | | | | | | |
| | | | precursor | | | | | | | |
| | | | 937230 | | | | | | | |
| | | | Homo | | | | | | | |
| | | | sapiens | | | | | | | |
| | | | cDNA | | | | | | | |
| | | | clone | | | | | | | |
| | | | 650401 3′ | | | | | | | |
| | | | similar to | | | | | | | |
| | | | contains | | | | | | | |
| | | | Alu repeti- | | | | | | | |
| | | | tive ele- | | | | | | | |
| | | | ment;. | | | | | | | |
| RC_AA449450_at | AAAPP | | zx05e04.s1 | 237 | A | 315 | A | NC | 1.3 | 0.07 |
| | | | Soares | | | | | | | |
| | | | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 785598 3′. | | | | | | | |

A classifiers
Gained in Duke A and Inc; NC in other Dukes;
Avg Diff in A > 300

| | | | | | | | | | | |
|---|---|---|---|---|---|---|---|---|---|---|
| RC_AA599199_at | APAAA | ALU seq. | ag34a11.s1 | 4 | A | 833 | P | I | ˜24.1 | 15.25 |
| | | | Jia bone | | | | | | | |
| | | | marrow | | | | | | | |
| | | | stroma | | | | | | | |
| | | | Homo | | | | | | | |
| | | | sapiens | | | | | | | |
| | | | cDNA | | | | | | | |
| | | | clone | | | | | | | |
| | | | 1091420 3′ | | | | | | | |
| | | | similar to | | | | | | | |
| | | | contains | | | | | | | |
| | | | Alu repeti- | | | | | | | |
| | | | tive ele- | | | | | | | |
| | | | ment; con- | | | | | | | |
| | | | tains ele- | | | | | | | |
| | | | ment | | | | | | | |
| | | | TAR1 | | | | | | | |
| | | | repetitive | | | | | | | |
| | | | element;. | | | | | | | |
| RC_R12694_at | APAAA | unnamed protein | yf40f05.s1 | 115 | A | 361 | P | I | 3 | 1.02 |
| | | product BAA91641, | Homo | | | | | | | |
| | | chrom 10 | sapiens | | | | | | | |
| | | | cDNA | | | | | | | |
| | | | clone | | | | | | | |
| | | | 129345 3′ | | | | | | | |
| | | | similar to | | | | | | | |
| | | | contains | | | | | | | |
| | | | Alu repeti- | | | | | | | |
| | | | tive ele- | | | | | | | |
| | | | ment;. | | | | | | | |
| RC_H91325_s_at | APAAA | aldolase B; aldolase B | yu96g02.s1 | 92 | A | 318 | P | I | 3.4 | 1.33 |
| | | (aa 1-364); chrom 9 | Homo | | | | | | | |
| | | | sapiens | | | | | | | |
| | | | cDNA | | | | | | | |
| | | | clone | | | | | | | |
| | | | 241106 3′ | | | | | | | |
| | | | similar to | | | | | | | |
| | | | gb: X02747 | | | | | | | |
| | | | FRUC- | | | | | | | |
| | | | TOSE-BIS- | | | | | | | |
| | | | PHOS- | | | | | | | |
| | | | PHATE | | | | | | | |
| | | | ALDO- | | | | | | | |
| | | | LASE B | | | | | | | |
| | | | (HUMAN);. | | | | | | | |
| RC_N51709_at | APAAA | chrom X | yy72e04.s1 | 466 | A | 440 | P | I | −1.1 | −0.01 |
| | | | Homo | | | | | | | |
| | | | sapiens | | | | | | | |
| | | | cDNA | | | | | | | |
| | | | clone | | | | | | | |
| | | | 279102 3′. | | | | | | | |
| RC_N72610_at | APAAA | — | za46h03.s1 | 502 | A | 531 | P | I | 1.1 | 0.01 |
| | | | Homo | | | | | | | |
| | | | sapiens | | | | | | | |
| | | | cDNA | | | | | | | |
| | | | clone | | | | | | | |
| | | | 295637 3′. | | | | | | | |

Lost in Duke A and Dec; NC in other Dukes
Avg Diff in N > 300

| | | | | | | | | | | |
|---|---|---|---|---|---|---|---|---|---|---|
| RC_N32411_f_at | PAPPP | Myc-associated zinc- | yw83b08.s1 | 4153 | P | 1319 | A | D | −3.5 | −5.06 |
| | | finger protein of hu- | Homo | | | | | | | |
| | | man islet; chrom 16 | sapiens | | | | | | | |
| | | | cDNA | | | | | | | |
| | | | clone | | | | | | | |
| | | | 258807 3′ | | | | | | | |
| | | | similar to | | | | | | | |
| | | | gb: M94046 | | | | | | | |
| | | | Human | | | | | | | |
| | | | zinc finger | | | | | | | |
| | | | protein | | | | | | | |
| | | | (HUMAN);. | | | | | | | |
| RC_AA243858_at | PAPPP | KIAA0882 protein | zr68c02.s1 | 389 | P | 77 | A | D | −5 | −2.67 |
| | | | Soares | | | | | | | |
| | | | NhHMPu | | | | | | | |
| | | | S1 Homo | | | | | | | |
| | | | sapiens | | | | | | | |
| | | | cDNA | | | | | | | |
| | | | clone | | | | | | | |
| | | | 668546 3′. | | | | | | | |
| RC_AA486283_at | PAPPP | ras-like protein; ras- | ab35h03.s1 | 4856 | P | 720 | A | D | −6.2 | −12.02 |
| | | related C3 botulinum | Strata- | | | | | | | |
| | | toxin substrate; | gene HeLa | | | | | | | |
| | | dJ20J23 | cell | | | | | | | |
| | | | s3937216 | | | | | | | |
| | | | Homo | | | | | | | |
| | | | sapiens | | | | | | | |
| | | | cDNA | | | | | | | |
| | | | clone | | | | | | | |
| | | | 842837 3′ | | | | | | | |
| | | | similar to | | | | | | | |
| | | | gb: M29870 | | | | | | | |
| | | | RAS- | | | | | | | |
| | | | RELATED | | | | | | | |
| | | | C3 BOTU- | | | | | | | |
| | | | LINUM | | | | | | | |
| | | | TOXIN | | | | | | | |
| | | | SUB- | | | | | | | |
| | | | STRATE 2 | | | | | | | |
| | | | (HUMAN);. | | | | | | | |
| RC_AA490930_at | PAPPP | chrom 18; KIAA1468 | aa46e04.s1 | 552 | P | 242 | A | D | −2.3 | −0.72 |
| | | protein | NCI CGAP_{—} | | | | | | | |
| | | | GCB1 | | | | | | | |
| | | | Homo | | | | | | | |
| | | | sapiens | | | | | | | |
| | | | cDNA | | | | | | | |
| | | | clone | | | | | | | |
| | | | IMAGE: | | | | | | | |
| | | | 823998 3′. | | | | | | | |

PPPPP; Inc in Duke A; NC in other Dukes
Avg Diff N >= 200; Fold change N to A >= 3x

| | | | | | | | | | | |
|---|---|---|---|---|---|---|---|---|---|---|
| RC_N69263_at | PPPPP | chrom 10; AK026414 | za38e10.s1 | 314 | P | 1239 | P | I | 3.4 | 2.35 |
| | | clone (only 108 nt | Homo | | | | | | | |
| | | hom) | sapiens | | | | | | | |
| | | | cDNA | | | | | | | |
| | | | clone | | | | | | | |
| | | | 294858 3′. | | | | | | | |
| RC_T15817_f_at | PPPPP | iNOS, inducible nitric | IB1935 | 484 | P | 1182 | P | I | 3.4 | 2.45 |
| | | oxide synthase | Infant | | | | | | | |
| | | | brain, | | | | | | | |
| | | | Bento | | | | | | | |
| | | | Soares | | | | | | | |
| | | | Homo | | | | | | | |
| | | | sapiens | | | | | | | |
| | | | cDNA | | | | | | | |
| | | | 3′ end | | | | | | | |
| | | | similar to | | | | | | | |
| | | | H. sapiens | | | | | | | |
| | | | inducible | | | | | | | |
| | | | nitric oxide | | | | | | | |
| | | | synthase | | | | | | | |
| | | | mRNA. | | | | | | | |

PPPPP; Dec in Duke A; NC in other Dukes
Avg Diff N >= 200; Fold change N to A >= 3x

| | | | | | | | | | | |
|---|---|---|---|---|---|---|---|---|---|---|
| RC_H54088_s_at | PPPPP | ribosomal protein L41 | yq88g11.s1 | 5317 | P | 1302 | P | D | −4.1 | −7.26 |
| | | | Homo | | | | | | | |
| | | | sapiens | | | | | | | |
| | | | cDNA | | | | | | | |
| | | | clone | | | | | | | |
| | | | 202916 3′. | | | | | | | |
| RC_H59052_f_at | PPPPP | fungal sterol-C5- | yr40g11.s1 | 5090 | P | 1086 | P | D | −4.2 | −7.05 |
| | | desaturase homolog; | Homo | | | | | | | |
| | | ORF; thymosin beta-4 | sapiens | | | | | | | |
| | | | cDNA | | | | | | | |
| | | | clone | | | | | | | |
| | | | 207812 3′ | | | | | | | |
| | | | similar to | | | | | | | |
| | | | gb: M1773 | | | | | | | |
| | | | 3 THY- | | | | | | | |
| | | | MOSIN | | | | | | | |
| | | | BETA-4 | | | | | | | |
| | | | (HUMAN);. | | | | | | | |
| RC_R49198_s_at | PPPPP | — | yg58h09.s1 | 4836 | P | 1115 | P | D | −4.3 | −7.61 |
| | | | Homo | | | | | | | |
| | | | sapiens | | | | | | | |
| | | | cDNA | | | | | | | |
| | | | clone | | | | | | | |
| | | | 37141 3′ | | | | | | | |
| | | | similar to | | | | | | | |
| | | | gb: | | | | | | | |
| | | | X6915040S | | | | | | | |
| | | | RIBOSO- | | | | | | | |
| | | | MAL | | | | | | | |
| | | | PROTEIN | | | | | | | |
| | | | S18 (HU- | | | | | | | |
| | | | MAN);. | | | | | | | |
| RC_T73572_f_at | PPPPP | ferritin L-chain; L | yc36c10.s1 | 5727 | P | 1853 | P | D | −3.1 | −4.59 |
| | | apoferritin | Homo | | | | | | | |
| | | | sapiens | | | | | | | |
| | | | cDNA | | | | | | | |
| | | | clone | | | | | | | |
| | | | 82770 3′ | | | | | | | |
| | | | similar to | | | | | | | |
| | | | gb: M10119 | | | | | | | |
| | | | FERRI- | | | | | | | |
| | | | TIN LIGHT | | | | | | | |
| | | | CHAIN | | | | | | | |
| | | | (HUMAN);. | | | | | | | |
| RC_AA477483_at | PPPPP | no matching est | zu44h02.s1 | 4494 | P | 1034 | P | D | −3.8 | −5.51 |
| | | | Soares | | | | | | | |
| | | | ovary | | | | | | | |
| | | | tumor | | | | | | | |
| | | | NbHOT | | | | | | | |
| | | | Homo | | | | | | | |
| | | | sapiens | | | | | | | |
| | | | cDNA | | | | | | | |
| | | | clone | | | | | | | |
| | | | 740883 3′ | | | | | | | |
| | | | similar to | | | | | | | |
| | | | SW: NU3M_{—} | | | | | | | |
| | | | HUMAN | | | | | | | |
| | | | P03897 | | | | | | | |
| | | | NADH-UBI- | | | | | | | |
| | | | QUINONE | | | | | | | |
| | | | OXIDO- | | | | | | | |
| | | | REDUC- | | | | | | | |
| | | | TASE | | | | | | | |
| | | | CHAIN 3;. | | | | | | | |

B classifiers
Gained in Duke B and Inc; NC in other Dukes
Avg Diff in B > 300

| | | | | | | | | | | |
|---|---|---|---|---|---|---|---|---|---|---|
| RC_T67463_s_at | AAPAA | cathepsin O2; X; K | yd12c04.s1 | 132 | A | 240 | A | NC | 1.8 | 0.24 |
| | | | Homo | | | | | | | |
| | | | sapiens | | | | | | | |
| | | | cDNA | | | | | | | |
| | | | clone | | | | | | | |
| | | | 66918 3′ | | | | | | | |
| | | | similar to | | | | | | | |
| | | | gb: X12451 | | | | | | | |
| | | | CATHEP- | | | | | | | |
| | | | SIN L | | | | | | | |
| | | | PRECUR- | | | | | | | |
| | | | SOR | | | | | | | |
| | | | (HUMAN);. | | | | | | | |
| RC_W94688_at | AAPAA | perilipin | ze04f12.s1 | 139 | A | 137 | A | NC | −1 | 0 |
| | | | Soares | | | | | | | |
| | | | fetal heart | | | | | | | |
| | | | NbHH19W | | | | | | | |
| | | | Homo | | | | | | | |
| | | | sapiens | | | | | | | |
| | | | cDNA | | | | | | | |
| | | | clone | | | | | | | |
| | | | 358031 3′. | | | | | | | |
| RC_AA126743_at | AAPAA | Z97200 PAC chrom | zk95d02.s1 | 402 | A | 384 | A | NC | 4.3 | 2.1 |
| | | 1q24; PMX1 homeo- | Soares | | | | | | | |
| | | box gene | pregnant | | | | | | | |
| | | | uterus | | | | | | | |
| | | | NbHPU | | | | | | | |
| | | | Homo | | | | | | | |
| | | | sapiens | | | | | | | |
| | | | cDNA | | | | | | | |
| | | | clone | | | | | | | |
| | | | 490563 3′. | | | | | | | |
| RC_AA236547_at | AAPAA | no homology | zs39a03.s1 | 223 | A | −151 | A | NC | −2.3 | −0.48 |
| | | | Soares | | | | | | | |
| | | | NhHMPu | | | | | | | |
| | | | S1 Homo | | | | | | | |
| | | | sapiens | | | | | | | |
| | | | cDNA | | | | | | | |
| | | | clone | | | | | | | |
| | | | 687532 3′. | | | | | | | |
| RC_AA255567_at | AAPAA | angiopoietin-related | zr85e11.s1 | 118 | A | 160 | A | NC | 1.3 | 0.05 |
| | | protein-2; angiopoietin- | Soares | | | | | | | |
| | | like 2 | NhHMPu | | | | | | | |
| | | | S1 Homo | | | | | | | |
| | | | sapiens | | | | | | | |
| | | | cDNA | | | | | | | |
| | | | clone | | | | | | | |
| | | | 682508 3′. | | | | | | | |
| RC_AA421256_at | AAPAA | — | zu06a01.s1 | 338 | A | 171 | A | NC | −2 | −0.38 |
| | | | Soares | | | | | | | |
| | | | testis NHT | | | | | | | |
| | | | Homo | | | | | | | |
| | | | sapiens | | | | | | | |
| | | | cDNA | | | | | | | |
| | | | clone | | | | | | | |
| | | | 731016 3′. | | | | | | | |

Lost in Duke B and Dec; NC in other Dukes
Avg Diff in N > 300

| | | | | | | | | | | |
|---|---|---|---|---|---|---|---|---|---|---|
| RC_D59847_at | PPAPP | proSAAS; granin-like | Human | 864 | P | 668 | P | NC | −1.3 | −0.1 |
| | | neuroendocrine pep- | fetal brain | | | | | | | |
| | | tide precursor | cDNA 3′- | | | | | | | |
| | | | end GEN- | | | | | | | |
| | | | 070G07. | | | | | | | |
| RC_F05038_at | PPAPP | polyamine modulated | H. sapiens | 1683 | P | 1460 | P | NC | −1.2 | −0.05 |
| | | factor-1; polyamine | partial | | | | | | | |
| | | modulated factor 1 | CDNA | | | | | | | |
| | | | sequence; | | | | | | | |
| | | | clone c- | | | | | | | |
| | | | 1gb09. | | | | | | | |
| RC_N41059_at | PPAPP | chrom 3 | yy53h01.s1 | 350 | P | 334 | P | NC | −1 | 0 |
| | | | Homo | | | | | | | |
| | | | sapiens | | | | | | | |
| | | | cDNA | | | | | | | |
| | | | clone | | | | | | | |
| | | | 277297 3′. | | | | | | | |
| RC_T23460_at | PPAPP | chrom 3; IFNAR2 | seq3026 | 613 | P | 560 | P | NC | −1.1 | −0.01 |
| | | 21q22.11 | Homo | | | | | | | |
| | | | sapiens | | | | | | | |
| | | | cDNA | | | | | | | |
| | | | clone | | | | | | | |
| | | | Hy18- | | | | | | | |
| | | | Ch13- | | | | | | | |
| | | | Charon40- | | | | | | | |
| | | | cDNA-76 | | | | | | | |
| | | | 3′. | | | | | | | |
| RC_W42789_at | PPAPP | chrom 8 AF268037 | zc25a06.s1 | 310 | P | 234 | P | NC | −1.3 | −0.07 |
| | | C8ORF4 protein | Soares | | | | | | | |
| | | (C8ORF4) chrom 8 | senescent | | | | | | | |
| | | ORF | fibroblasts | | | | | | | |
| | | | NbHSF | | | | | | | |
| | | | Homo | | | | | | | |
| | | | sapiens | | | | | | | |
| | | | cDNA | | | | | | | |
| | | | clone | | | | | | | |
| | | | 323314 3′. | | | | | | | |
| RC_AA460017_l_{—} | PPAPP | BAC clone chrom 16 | zx49h08.s1 | 1370 | P | 703 | P | NC | −1.9 | −0.73 |
| at | | | Soares | | | | | | | |
| | | | testis NHT | | | | | | | |
| | | | Homo | | | | | | | |
| | | | sapiens | | | | | | | |
| | | | cDNA | | | | | | | |
| | | | clone | | | | | | | |
| | | | 795615 3′. | | | | | | | |
| RC_AA482127_at | PPAPP | KIAA1142 protein | zv43g10.s1 | 356 | P | 288 | P | NC | −1.2 | −0.04 |
| | | | Soares | | | | | | | |
| | | | ovary | | | | | | | |
| | | | tumor | | | | | | | |
| | | | NbHOT | | | | | | | |
| | | | Homo | | | | | | | |
| | | | sapiens | | | | | | | |
| | | | cDNA | | | | | | | |
| | | | clone | | | | | | | |
| | | | 756450 3′. | | | | | | | |
| RC_AA504806_at | PPAPP | chrom 2 AF052107 | aa64a10.s1 | 421 | P | 384 | P | NC | −1.4 | −0.12 |
| | | clone 23620 mRNA | NCI CGAP_{—} | | | | | | | |
| | | sequence | GCB1 | | | | | | | |
| | | | Homo | | | | | | | |
| | | | sapiens | | | | | | | |
| | | | cDNA | | | | | | | |
| | | | clone | | | | | | | |
| | | | IMAGE: | | | | | | | |
| | | | 825690 3′. | | | | | | | |

Inc in Duke B; NC in other Dukes
Avg Diff N >= 200; Fold change N to A >= 3x

| | | | | | | | | | | |
|---|---|---|---|---|---|---|---|---|---|---|
| RC_AA386386_s_{—} | PPPPP | — | EST81368 | 1672 | P | 2253 | P | NC | 1.4 | 0.3 |
| at | | | Prostate | | | | | | | |
| | | | gland I | | | | | | | |
| | | | Homo | | | | | | | |
| | | | sapiens | | | | | | | |
| | | | cDNA 3′ | | | | | | | |
| | | | end similar | | | | | | | |
| | | | to similar | | | | | | | |
| | | | to prolyl 4- | | | | | | | |
| | | | hydroxy- | | | | | | | |
| | | | lase, beta | | | | | | | |
| | | | sub- | | | | | | | |
| | | | unit/protein | | | | | | | |
| | | | disulfide | | | | | | | |
| | | | isomer- | | | | | | | |
| | | | ase/thyroid | | | | | | | |
| | | | hormone- | | | | | | | |
| | | | binding | | | | | | | |
| | | | protein. | | | | | | | |
| RC_AA452549_at | PPPPP | PR01659; hypotheti- | zx35f04.s1 | 400 | P | 368 | P | NC | −1.1 | −0.01 |
| | | cal protein chrom 11 | Soares | | | | | | | |
| | | | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 788479 3′. | | | | | | | |

Dec in Duke B; NC in other dukes
Avg Diff N >= 200; Fold change N to B >= 3x

| | | | | | | | | | | |
|---|---|---|---|---|---|---|---|---|---|---|
| RC_T90037_at | PPPPP | unnamed protein | ye13d01.s1 | 948 | P | 568 | P | NC | −1.7 | −.036 |
| | | product, chrom 4 | Homo | | | | | | | |
| | | | sapiens | | | | | | | |
| | | | cDNA | | | | | | | |
| | | | clone | | | | | | | |
| | | | 117601 3′. | | | | | | | |
| RC_AA432130_at | PPPPP | KIAA0867 protein, | zw69f10.s1 | 1007 | P | 475 | P | NC | −2.1 | −0.8 |
| | | chrom 12 | Soares | | | | | | | |
| | | | testis NHT | | | | | | | |
| | | | Homo | | | | | | | |
| | | | sapiens | | | | | | | |
| | | | cDNA | | | | | | | |
| | | | clone | | | | | | | |
| | | | 781483 3′ | | | | | | | |
| | | | similar to | | | | | | | |
| | | | contains | | | | | | | |
| | | | Alu repeti- | | | | | | | |
| | | | tive ele- | | | | | | | |
| | | | ment; con- | | | | | | | |
| | | | tains ele- | | | | | | | |
| | | | ment | | | | | | | |
| | | | PTR7 | | | | | | | |
| | | | repetitive | | | | | | | |
| | | | element;. | | | | | | | |

C classifiers
Gained in Duke C and Inc; NC in other Dukes
Avg Diff in C > 300

| | | | | | | | | | | |
|---|---|---|---|---|---|---|---|---|---|---|
| RC_D45556_at | AAAPA | chrom 15; AL390085 | Human | 391 | A | 222 | A | NC | −1.8 | −0.28 |
| | | clone | adult lung | | | | | | | |
| | | | 3′ directed | | | | | | | |
| | | | Mbol | | | | | | | |
| | | | CDNA, | | | | | | | |
| | | | HUMGS02743, | | | | | | | |
| | | | clone | | | | | | | |
| | | | Ig1160. | | | | | | | |
| RC_W86214_at | AAAPA | | zh54a11.s1 | 88 | A | 132 | A | NC | 1.6 | 0.12 |
| | | | Soares | | | | | | | |
| | | | fetal liver | | | | | | | |
| | | | spleen | | | | | | | |
| | | | 1NFLS S1 | | | | | | | |
| | | | Homo | | | | | | | |
| | | | sapiens | | | | | | | |
| | | | cDNA | | | | | | | |
| | | | clone | | | | | | | |
| | | | 415868 3′. | | | | | | | |
| RC_AA039439_s_{—} | AAAPA | novel gene KIAA0134 | zk39a11.s1 | 134 | A | 608 | A | NC | 3.8 | 1.94 |
| at | | protein 19q13.3 | Soares | | | | | | | |
| | | | pregnant | | | | | | | |
| | | | uterus | | | | | | | |
| | | | NbHPU | | | | | | | |
| | | | Homo | | | | | | | |
| | | | sapiens | | | | | | | |
| | | | cDNA | | | | | | | |
| | | | clone | | | | | | | |
| | | | 485180 3′. | | | | | | | |
| RC_AA128935_at | AAAPA | | zo08e09.s1 | 162 | A | 270 | A | NC | 1.7 | 0.19 |
| | | | Strata- | | | | | | | |
| | | | gene neuro | | | | | | | |
| | | | epithelium | | | | | | | |
| | | | NT2RAMI | | | | | | | |
| | | | 937234 | | | | | | | |
| | | | Homo | | | | | | | |
| | | | sapiens | | | | | | | |
| | | | cDNA | | | | | | | |
| | | | clone | | | | | | | |
| | | | 567112 3′. | | | | | | | |
| RC_AA134158_s_{—} | AAAPA | class I homeodomain; | zo18b11.s1 | 255 | A | 200 | A | NC | 3.7 | 1.17 |
| at | | homeobox protein, | Strata- | | | | | | | |
| | | chrom 7 | gene colon | | | | | | | |
| | | | (#937204) | | | | | | | |
| | | | Homo | | | | | | | |
| | | | sapiens | | | | | | | |
| | | | cDNA | | | | | | | |
| | | | clone | | | | | | | |
| | | | 587229 3′ | | | | | | | |
| | | | similar to | | | | | | | |
| | | | gb: S41211 | | | | | | | |
| | | | HOMEO- | | | | | | | |
| | | | BOX | | | | | | | |
| | | | PROTEIN | | | | | | | |
| | | | HOX-A10 | | | | | | | |
| | | | (HUMAN);. | | | | | | | |
| RC_AA232646_at | AAAPA | chrom 17, AF266756 | zr45f08.s1 | 123 | A | 238 | A | NC | 1.9 | 0.3 |
| | | sphingosine kinase | Soares | | | | | | | |
| | | (SPHK1 | NhHMPu | | | | | | | |
| | | | S1 Homo | | | | | | | |
| | | | sapiens | | | | | | | |
| | | | cDNA | | | | | | | |
| | | | clone | | | | | | | |
| | | | 666375 3′. | | | | | | | |
| RC_AA401184_at | AAAPA | no homology | zu52b06.s1 | 348 | A | 62 | A | NC | ˜−1.2 | −0.01 |
| | | | Soares | | | | | | | |
| | | | ovary | | | | | | | |
| | | | tumor | | | | | | | |
| | | | NbHOT | | | | | | | |
| | | | Homo | | | | | | | |
| | | | sapiens | | | | | | | |
| | | | cDNA | | | | | | | |
| | | | clone | | | | | | | |
| | | | 741587 3′. | | | | | | | |
| RC_AA436840_at | AAAPA | | zv57g11.s1 | 399 | A | −13 | A | NC | ˜−6.1 | −2 |
| | | | Soares | | | | | | | |
| | | | testis NHT | | | | | | | |
| | | | Homo | | | | | | | |
| | | | sapiens | | | | | | | |
| | | | cDNA | | | | | | | |
| | | | clone | | | | | | | |
| | | | 757796 3′. | | | | | | | |
| RC_AA488655_at | AAAPA | | ab40c03.s1 | −4 | A | 176 | A | NC | ˜6.8 | 2.59 |
| | | | Strata- | | | | | | | |
| | | | gene HeLa | | | | | | | |
| | | | cell s3 | | | | | | | |
| | | | 937216 | | | | | | | |
| | | | Homo | | | | | | | |
| | | | sapiens | | | | | | | |
| | | | cDNA | | | | | | | |
| | | | clone | | | | | | | |
| | | | 843268 3′. | | | | | | | |

L st in Duke C and Dec; NC in other Dukes
Avg Diff in N > 300

| | | | | | | | | | | |
|---|---|---|---|---|---|---|---|---|---|---|
| RC_N30231_at | PPPAP | Lsm4 protein; U6 | yw64c08.s1 | 1496 | P | 885 | P | NC | −2.2 | −1.26 |
| | | snRNA-associated | Homo | | | | | | | |
| | | Sm-like protein LSm4; | sapiens | | | | | | | |
| | | glycine-rich protein | cDNA | | | | | | | |
| | | | clone | | | | | | | |
| | | | 257006 3′ | | | | | | | |
| | | | similar to | | | | | | | |
| | | | contains | | | | | | | |
| | | | Alu repeti- | | | | | | | |
| | | | tive ele- | | | | | | | |
| | | | ment;. | | | | | | | |
| RC_W73790_f_at | PPPAP | immunoglobulin- | zd50g12.s1 | 3320 | P | 1638 | P | NC | −2 | −1.27 |
| | | related protein 14.1; | Soares | | | | | | | |
| | | lambda L-chain C | fetal heart | | | | | | | |
| | | region; omega protein, | NbHH19W | | | | | | | |
| | | chrom 22 | Homo | | | | | | | |
| | | | sapiens | | | | | | | |
| | | | cDNA | | | | | | | |
| | | | clone | | | | | | | |
| | | | 344134 3′ | | | | | | | |
| | | | similar to | | | | | | | |
| | | | gb: M27749 | | | | | | | |
| | | | IMMU- | | | | | | | |
| | | | NOGLOB- | | | | | | | |
| | | | ULIN- | | | | | | | |
| | | | RELATED | | | | | | | |
| | | | 14.1 | | | | | | | |
| | | | PROTEIN | | | | | | | |
| | | | PRECUR- | | | | | | | |
| | | | SOR | | | | | | | |
| | | | (HUMAN);. | | | | | | | |
| RC_AA412184_at | PPPAP | chrom 1p36; d89060 | zt93f09.s1 | 714 | P | 570 | P | NC | −1.4 | −0.15 |
| | | dolichyl- | Soares | | | | | | | |
| | | diphosphooligosaccha- | testis NHT | | | | | | | |
| | | ride-protein glycosyl- | Homo | | | | | | | |
| | | transferase | sapiens | | | | | | | |
| | | | cDNA | | | | | | | |
| | | | clone | | | | | | | |
| | | | 729929 3′ | | | | | | | |
| | | | similar to | | | | | | | |
| | | | WP: EEED | | | | | | | |
| | | | 8.9 | | | | | | | |
| | | | CE01893;. | | | | | | | |
| RC_AA521303_at | PPPAP | methionine adenosyl- | aa79f11.s1 | 777 | P | 760 | P | NC | −1 | 0 |
| | | transferase regulatory | NCI_CGAP_{—} | | | | | | | |
| | | beta subunit; dTDP-4- | GCB1 | | | | | | | |
| | | keto-6-deoxy-D- | Homo | | | | | | | |
| | | glucose 4-reductase, | sapiens | | | | | | | |
| | | chrom 5 | cDNA | | | | | | | |
| | | | clone | | | | | | | |
| | | | IMAGE: | | | | | | | |
| | | | 827181 3′. | | | | | | | |

Inc in Duke C; NC in other Dukes
Avg Diff N >= 200; Fold change N to C >= 3x

| | | | | | | | | | | |
|---|---|---|---|---|---|---|---|---|---|---|
| RC_AA181902_at | PPPPP | AC007201 on chrom | zp63d06.s1 | 478 | P | 679 | P | NC | 1.6 | 0.29 |
| | | 19 (only 80 nt hom) | Strata- | | | | | | | |
| | | | gene | | | | | | | |
| | | | endothelial | | | | | | | |
| | | | cell | | | | | | | |
| | | | 937223 | | | | | | | |
| | | | Homo | | | | | | | |
| | | | sapiens | | | | | | | |
| | | | cDNA | | | | | | | |
| | | | clone | | | | | | | |
| | | | 624875 3′ | | | | | | | |
| | | | similar to | | | | | | | |
| | | | contains | | | | | | | |
| | | | Alu repeti- | | | | | | | |
| | | | tive ele- | | | | | | | |
| | | | ment;. | | | | | | | |

Dec in Duke C; NC in other Dukes
Avg Diff N >= 200; Fold change N to D >= 3x

| | | | | | | | | | | |
|---|---|---|---|---|---|---|---|---|---|---|
| RC_AA461174_at | PPPPP | 8p21.3-p22 AB020860 | zx70c04.s1 | 327 | P | 198 | P | NC | −1.5 | −0.12 |
| | | anti-oncogene | Soares | | | | | | | |
| | | | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 796806 3′. | | | | | | | |
| AA393432_s_at | PPPPP | chrom 2, Unknown; | zt71a04.r1 | 319 | P | 222 | P | NC | −1.4 | −0.11 |
| | | unnamed protein | Soares | | | | | | | |
| | | product AAD20029 | testis NHT | | | | | | | |
| | | | Homo | | | | | | | |
| | | | sapiens | | | | | | | |
| | | | cDNA | | | | | | | |
| | | | clone | | | | | | | |
| | | | 727758 5′. | | | | | | | |

D classifier
Gained in Duke D and Inc; NC in other Dukes
Avg Diff in D > 300

| | | | | | | | | | | |
|---|---|---|---|---|---|---|---|---|---|---|
| RC_N91920_at | AAAAP | chrom 16p12-p11.2; | zb48e07.s1 | −76 | A | 58 | A | NC | ˜4.7 | 0.62 |
| | | XN_007994 retinobla- | Soares | | | | | | | |
| | | stoma binding protein | fetal lung | | | | | | | |
| | | | NbHL19W | | | | | | | |
| | | | Homo | | | | | | | |
| | | | sapiens | | | | | | | |
| | | | cDNA | | | | | | | |
| | | | clone | | | | | | | |
| | | | 306852 3′. | | | | | | | |
| RC_AA621601_at | AAAAP | chrom 17 XM_009868 | af47g08.s1 | −46 | A | 232 | A | NC | ˜−3.5 | −0.18 |
| | | RAB36 ARS oncogene | Soares | | | | | | | |
| | | family | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 1034846 3′ | | | | | | | |
| | | | similar to | | | | | | | |
| | | | TR: | | | | | | | |
| | | | G240986 | | | | | | | |
| | | | G240986 | | | | | | | |
| | | | LMW G- | | | | | | | |
| | | | PROTEIN. | | | | | | | |
| | | | ;. | | | | | | | |

Avg Diff in N > 500
Lost in Duke D and Dec; NC in other Dukes

| | | | | | | | | | | |
|---|---|---|---|---|---|---|---|---|---|---|
| RC_R72886_s_at | PPPPA | KIAA0422; adenylyl | yl10f04.s1 | 1768 | P | 880 | P | NC | −2 | −0.91 |
| | | cyclase type VI, chrom | Homo | | | | | | | |
| | | 12 | sapiens | | | | | | | |
| | | | cDNA | | | | | | | |
| | | | clone | | | | | | | |
| | | | 157855 3′. | | | | | | | |
| RC_AA026030_at | PPPPA | chrom 1 | ze84d01.s1 | 1478 | P | 1163 | P | NC | −1.3 | −0.11 |
| | | | Soares | | | | | | | |
| | | | fetal heart | | | | | | | |
| | | | NbHH19W | | | | | | | |
| | | | Homo | | | | | | | |
| | | | sapiens | | | | | | | |
| | | | cDNA | | | | | | | |
| | | | clone | | | | | | | |
| | | | 365665 3′ | | | | | | | |
| | | | similar to | | | | | | | |
| | | | PIR: A48764 | | | | | | | |
| | | | A48764 | | | | | | | |
| | | | calpain;. | | | | | | | |
| RC_Z39006_at | PPPPA | hypothetical protein, | H. sapiens | 1251 | P | 691 | P | NC | −1.4 | −0.14 |
| | | chrom 17 | partial | | | | | | | |
| | | | cDNA | | | | | | | |
| | | | sequence; | | | | | | | |
| | | | clone c- | | | | | | | |
| | | | Owe07. | | | | | | | |
| RC_AA435908_at | PPPPA | chrom 19; ac011491 | zt87a11.s1 | 1173 | P | 494 | P | NC | −1.6 | −0.28 |
| | | clone and 20 nt hom. | Soares | | | | | | | |
| | | RAB2, RAS oncogene | testis NHT | | | | | | | |
| | | family | Homo | | | | | | | |
| | | | sapiens | | | | | | | |
| | | | cDNA | | | | | | | |
| | | | clone | | | | | | | |
| | | | 729308 3′. | | | | | | | |
| RC_AA057829_s_{—} | PPPPA | growth-arrest-specific | zl95c02.s1 | 1136 | P | 534 | P | NC | −1.5 | −0.26 |
| at | | protein; growth arrest- | Stratagene | | | | | | | |
| | | specific 6; AXL stimu- | corneal | | | | | | | |
| | | latory factor, chrom 13 | stroma | | | | | | | |
| | | | (#937222) | | | | | | | |
| | | | Homo | | | | | | | |
| | | | sapiens | | | | | | | |
| | | | cDNA | | | | | | | |
| | | | clone | | | | | | | |
| | | | 512354 3′ | | | | | | | |
| | | | similar to | | | | | | | |
| | | | TR: | | | | | | | |
| | | | G401767 | | | | | | | |
| | | | G401767 | | | | | | | |
| | | | GROWTH- | | | | | | | |
| | | | ARREST- | | | | | | | |
| | | | SPECIFIC | | | | | | | |
| | | | PROTEIN.;. | | | | | | | |
| RC_R72087_at | PPPPA | chrom 5 EST; hom to | yj87e06.s1 | 923 | P | 768 | P | NC | −1.2 | −0.06 |
| | | chrom 20 AL356652 | Homo | | | | | | | |
| | | clone | sapiens | | | | | | | |
| | | | cDNA | | | | | | | |
| | | | clone | | | | | | | |
| | | | 155746 3′. | | | | | | | |
| RC_H04242_at | PPPPA | ras related protein | yj46c08.s1 | 869 | P | 661 | P | NC | −1.1 | −0.01 |
| | | Rab5b; RAB5B, mem- | Homo | | | | | | | |
| | | ber RAS oncogene | sapiens | | | | | | | |
| | | family | cDNA | | | | | | | |
| | | | clone | | | | | | | |
| | | | 151790 3′. | | | | | | | |
| RC_R97304_f_at | PPPPA | HLA-drb5; cell surface | yq52c02.s1 | 858 | P | 1822 | P | NC | 1.5 | 0.25 |
| | | glycoprotein; MHC | Homo | | | | | | | |
| | | HLA-DR-beta chain | sapiens | | | | | | | |
| | | precursor chrom 6 | cDNA | | | | | | | |
| | | | clone | | | | | | | |
| | | | 199394 3′ | | | | | | | |
| | | | similar to | | | | | | | |
| | | | gb: M33600 | | | | | | | |
| | | | HLA | | | | | | | |
| | | | CLASS II | | | | | | | |
| | | | HISTO- | | | | | | | |
| | | | COMPATI- | | | | | | | |
| | | | BILITY | | | | | | | |
| | | | ANTIGEN, | | | | | | | |
| | | | DR-1 | | | | | | | |
| | | | BETA | | | | | | | |
| | | | CHAIN | | | | | | | |
| | | | (HUMAN);. | | | | | | | |
| RC_N48609_at | PPPPA | chrom 11; AC004584 | yy74f08.s1 | 770 | P | 612 | P | NC | −1.1 | −0.02 |
| | | chrom 17 | Homo | | | | | | | |
| | | | sapiens | | | | | | | |
| | | | cDNA | | | | | | | |
| | | | clone | | | | | | | |
| | | | 279303 3′. | | | | | | | |
| RC_W86850_f_at | PPPPA | chrom 22 ? X96924 | zh59d02.s1 | 644 | P | 273 | P | NC | −1.4 | −0.09 |
| | | mitochondrial citrate | Soares | | | | | | | |
| | | tranbsport region | fetal liver | | | | | | | |
| | | | spleen | | | | | | | |
| | | | 1NFLS S1 | | | | | | | |
| | | | Homo | | | | | | | |
| | | | sapiens | | | | | | | |
| | | | cDNA | | | | | | | |
| | | | clone | | | | | | | |
| | | | 416355 3′. | | | | | | | |
| RC_AA130603_at | PPPPA | ak024908 clone | zo10a01.s1 | 640 | P | 568 | P | NC | −1.1 | −0.02 |
| | | | Strata- | | | | | | | |
| | | | gene neuro | | | | | | | |
| | | | epithelium | | | | | | | |
| | | | NT2RAMI | | | | | | | |
| | | | 937234 | | | | | | | |
| | | | Homo | | | | | | | |
| | | | sapiens | | | | | | | |
| | | | cDNA | | | | | | | |
| | | | clone | | | | | | | |
| | | | 567240 3′. | | | | | | | |
| RC_AA479610_at | PPPPA | singleton ak025344 | zu31a12.s1 | 640 | P | 571 | P | NC | −1.1 | −0.02 |
| | | clone | Soares | | | | | | | |
| | | | ovary | | | | | | | |
| | | | tumor | | | | | | | |
| | | | NbHOT | | | | | | | |
| | | | Homo | | | | | | | |
| | | | sapiens | | | | | | | |
| | | | cDNA | | | | | | | |
| | | | clone | | | | | | | |
| | | | 739582 3′ | | | | | | | |
| | | | similar to | | | | | | | |
| | | | contains | | | | | | | |
| | | | Alu repeti- | | | | | | | |
| | | | tive ele- | | | | | | | |
| | | | ment;. | | | | | | | |
| RC_AA490593_l_at | PPPPA | chrom 17 ? Synapto- | aa47e09.s1 | 540 | P | 536 | P | NC | −1.3 | −0.07 |
| | | brevin2 (VAMP2) | NCI_CGAP_{—} | | | | | | | |
| | | AF135372 | GCB1 | | | | | | | |
| | | | Homo | | | | | | | |
| | | | sapiens | | | | | | | |
| | | | cDNA | | | | | | | |
| | | | clone | | | | | | | |
| | | | IMAGE: | | | | | | | |
| | | | 824104 3′. | | | | | | | |
| RC_AA054321_s_{—} | PPPPA | 6p21 HLA class i | zl68c01.s1 | 509 | P | 472 | P | NC | −1.1 | −0.01 |
| at | | region; AC004202 | Stratagene | | | | | | | |
| | | clone | colon | | | | | | | |
| | | | (#937204) | | | | | | | |
| | | | Homo | | | | | | | |
| | | | sapiens | | | | | | | |
| | | | cDNA | | | | | | | |
| | | | clone | | | | | | | |
| | | | 509760 3′. | | | | | | | |

Inc in Duke D; NC in other Dukes
Avg Diff N >= 200; Fold change N to A >= 3x

| | | | | | | | | | | |
|---|---|---|---|---|---|---|---|---|---|---|
| D79052_s_at | PPPPP | Sec61 gamma | Human | 772 | P | 680 | P | NC | −1.3 | −0.08 |
| | | | placenta | | | | | | | |
| | | | cDNA 5′- | | | | | | | |
| | | | end GEN- | | | | | | | |
| | | | 530B11. | | | | | | | |
| RC_T40439_s_at | PPPPP | U2 small nuclear | ya01c08.s1 | 673 | P | 911 | P | NC | 1.2 | 0.06 |
| | | ribonucleoprotein B″; | Homo | | | | | | | |
| | | dJ705D16.1 | sapiens | | | | | | | |
| | | | cDNA | | | | | | | |
| | | | clone | | | | | | | |
| | | | 60206 3′ | | | | | | | |
| | | | similar to | | | | | | | |
| | | | SP: RU2B_{—} | | | | | | | |
| | | | HUMAN | | | | | | | |
| | | | P08579 | | | | | | | |
| | | | U2 SMALL | | | | | | | |
| | | | NUCLEAR | | | | | | | |
| | | | RIBONU- | | | | | | | |
| | | | CLEOPRO- | | | | | | | |
| | | | TEIN,. | | | | | | | |
| RC_AA251829_at | PPPPP | CGI-29 protein | zs09c12.s1 | 202 | P | 670 | P | NC | 2.8 | 1.19 |
| | | | NCI_CGAP_{—} | | | | | | | |
| | | | GCB1 | | | | | | | |
| | | | Homo | | | | | | | |
| | | | sapiens | | | | | | | |
| | | | cDNA | | | | | | | |
| | | | clone | | | | | | | |
| | | | IMAGE: | | | | | | | |
| | | | 684694 3′. | | | | | | | |

Dec in Duke D; NC in other Dukes
Avg Diff N >= 500; Fold change N to D >= −5x

| | | | | | | | | | | |
|---|---|---|---|---|---|---|---|---|---|---|
| RC_D60328_at | PPPPP | chrom 6, unknown; | Human | 1345 | P | 731 | P | NC | −1.7 | −0.4 |
| | | ring finger protein 5 | fetal brain | | | | | | | |
| | | | cDNA 3′- | | | | | | | |
| | | | end GEN- | | | | | | | |
| | | | 099F03. | | | | | | | |
| RC_H96850_at | PPPPP | oligosaccharyltransfer- | yw03b12.s1 | 4876 | P | 3348 | P | NC | −1.5 | −0.45 |
| | | ase d89060 1p36.1 | Soares | | | | | | | |
| | | (also C-class) | melano- | | | | | | | |
| | | | cyte | | | | | | | |
| | | | 2NbHM | | | | | | | |
| | | | Homo | | | | | | | |
| | | | sapiens | | | | | | | |
| | | | cDNA | | | | | | | |
| | | | clone | | | | | | | |
| | | | 251135 3′ | | | | | | | |
| | | | similar to | | | | | | | |
| | | | contains | | | | | | | |
| | | | Alu repeti- | | | | | | | |
| | | | tive ele- | | | | | | | |
| | | | ment;. | | | | | | | |
| RC_127444_at | PPPPP | chrom 1 no homology | zn92e09.s1 | 626 | P | 353 | P | NC | −1.8 | −0.36 |
| | | | Strata- | | | | | | | |
| | | | gene lung | | | | | | | |
| | | | carcinoma | | | | | | | |
| | | | 937218 | | | | | | | |
| | | | Homo | | | | | | | |
| | | | sapiens | | | | | | | |
| | | | cDNA | | | | | | | |
| | | | clone | | | | | | | |
| | | | 565672 3′ | | | | | | | |
| | | | similar to | | | | | | | |
| | | | TR: | | | | | | | |
| | | | G563829 | | | | | | | |
| | | | G563829 | | | | | | | |
| | | | FATTY | | | | | | | |
| | | | ACID | | | | | | | |
| | | | TRANS- | | | | | | | |
| | | | PORT | | | | | | | |
| | | | PROTEIN.;. | | | | | | | |
| RC_AA242824_at | PPPPP | chrom 11; ac005233 | zr65e11.s1 | 710 | P | 178 | P | NC | −2.2 | −0.52 |
| | | PAC clone chrom 22 | Soares | | | | | | | |
| | | | NhHMPu | | | | | | | |
| | | | S1 Homo | | | | | | | |
| | | | sapiens | | | | | | | |
| | | | cDNA | | | | | | | |
| | | | clone | | | | | | | |
| | | | 668300 3′. | | | | | | | |
| AA405775_s_at | PPPPP | similar to CAA16821 | zu57c10.r1 | 1047 | P | 692 | P | NC | −1.5 | −0.25 |
| | | (PID: g3255952) | Soares | | | | | | | |
| | | | ovary | | | | | | | |
| | | | tumor | | | | | | | |
| | | | NbHOT | | | | | | | |
| | | | Homo | | | | | | | |
| | | | sapiens | | | | | | | |
| | | | cDNA | | | | | | | |
| | | | clone | | | | | | | |
| | | | 742098 5′. | | | | | | | |

AB classifiers
Gained in Duke AB and Inc; NC in other Dukes
Avg Diff in AB >= 300

| | | | | | | | | | | |
|---|---|---|---|---|---|---|---|---|---|---|
| RC_AA136269_at | APPAA | HSPC314, chrom 12 | zk93b07.s1 | 184 | A | 319 | P | I | 2.9 | 1.25 |
| | | | Soares | | | | | | | |
| | | | pregnant | | | | | | | |
| | | | uterus | | | | | | | |
| | | | NbHPU | | | | | | | |
| | | | Homo | | | | | | | |
| | | | sapiens | | | | | | | |
| | | | cDNA | | | | | | | |
| | | | clone | | | | | | | |
| | | | 490357 3′. | | | | | | | |

Avg Diff in N > 300
Lost in Duke AB and Dec; NC in other Dukes

| | | | | | | | | | | |
|---|---|---|---|---|---|---|---|---|---|---|
| RC_T40895_at | PAAPP | protein tyrosine phos- | ya13f02.s1 | 1154 | P | 244 | A | D | −3.1 | −1.64 |
| | | phatase PTPCAAX1; | Homo | | | | | | | |
| | | protein tyrosine phos- | sapiens | | | | | | | |
| | | phatase hPRL-1N; iva- | cDNA | | | | | | | |
| | | 1, chrom 6 | clone | | | | | | | |
| | | | 61371 3′. | | | | | | | |
| RC_AA424400_at | PAAPP | chrom 14 | zv82e05.s | 344 | P | 123 | A | D | −2.8 | −0.91 |
| | | | 1 Soares | | | | | | | |
| | | | total fetus | | | | | | | |
| | | | Nb2HF8 | | | | | | | |
| | | | 9w Homo | | | | | | | |
| | | | sapiens | | | | | | | |
| | | | cDNA | | | | | | | |
| | | | clone | | | | | | | |
| | | | 760160 3′. | | | | | | | |

PPPPP; Inc in Duke AB
Avg Diff AB >= 300; Fold change N to AB >= 5x

| | | | | | | | | | | |
|---|---|---|---|---|---|---|---|---|---|---|
| RC_N22015_at | PPPPP | unnamed protein | yw31h10.s1 | 638 | P | 3335 | P | I | 5.2 | 8.09 |
| | | product, chrom 17; | Homo | | | | | | | |
| | | BC class. | sapiens | | | | | | | |
| | | | cDNA | | | | | | | |
| | | | clone | | | | | | | |
| | | | 253891 3′. | | | | | | | |
| RC_N50971_at | PPPPP | — | yy94b07.s1 | 129 | P | 1031 | P | I | 10 | 10.43 |
| | | | Homo | | | | | | | |
| | | | sapiens | | | | | | | |
| | | | CDNA | | | | | | | |
| | | | clone | | | | | | | |
| | | | 281173 3′. | | | | | | | |
| RC_AA194833_at | PPPPP | senescence- | zr31b04.s1 | 185 | P | 999 | P | I | 5.7 | 5.26 |
| | | associated epithelial | Soares | | | | | | | |
| | | membrane protein; | NhHMPu | | | | | | | |
| | | Claudin-1, chrom 3 | S1 Homo | | | | | | | |
| | | | sapiens | | | | | | | |
| | | | cDNA | | | | | | | |
| | | | clone | | | | | | | |
| | | | 664975 3′. | | | | | | | |
| RC_AA393803_at | PPPPP | chrom 6 | zv64c05.s1 | 69 | P | 310 | P | I | 5.7 | 2.8 |
| | | | Soares | | | | | | | |
| | | | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 758408 3′. | | | | | | | |

BC classifiers
Gained in Duke BC and Inc; NC in other Dukes
Avg Diff in BC > 300

| | | | | | | | | | | |
|---|---|---|---|---|---|---|---|---|---|---|
| RC_AA148923_at | AAPPA | DEPP; decidual pro- | zl27g11.s1 | 233 | A | 212 | A | NC | 1.2 | 0.04 |
| | | tein induced by proges- | Soares | | | | | | | |
| | | terone; hypothetical | pregnant | | | | | | | |
| | | protein, chrom 10 | uterus | | | | | | | |
| | | | NbHPU | | | | | | | |
| | | | Homo | | | | | | | |
| | | | sapiens | | | | | | | |
| | | | cDNA | | | | | | | |
| | | | clone | | | | | | | |
| | | | 503204 3′. | | | | | | | |

Lost in Duke BC and Dec
Avg Diff in N > 300

| | | | | | | | | | | |
|---|---|---|---|---|---|---|---|---|---|---|
| RC_N91919_s_at | PPAAP | mineralocorticoid | zb48e05.s1 | 618 | P | 206 | P | D | −2.4 | −0.82 |
| | | receptor; mineralocor- | Soares | | | | | | | |
| | | ticoid receptor (al- | fetal lung | | | | | | | |
| | | dosterone receptor); | NbHL19W | | | | | | | |
| | | chrom 4 | Homo | | | | | | | |
| | | | sapiens | | | | | | | |
| | | | cDNA | | | | | | | |
| | | | clone | | | | | | | |
| | | | 306848 3′ | | | | | | | |
| | | | similar to | | | | | | | |
| | | | gb: M16801 | | | | | | | |
| | | | MINE- | | | | | | | |
| | | | RALO- | | | | | | | |
| | | | CORTI- | | | | | | | |
| | | | COID | | | | | | | |
| | | | RECEP- | | | | | | | |
| | | | TOR | | | | | | | |
| | | | (HUMAN);. | | | | | | | |
| RC_T99196_s_at | PPAAP | chrom 5 AF010235 | ye62f11.s1 | 529 | P | 274 | P | D | −2.2 | −0.67 |
| | | Homo sapiens mRNA | Homo | | | | | | | |
| | | from chromosome | sapiens | | | | | | | |
| | | 5q31-33 region | cDNA | | | | | | | |
| | | | clone | | | | | | | |
| | | | 122349 3′. | | | | | | | |
| RC_W72366_at | PPAAP | chrom 1 clone RP11- | Zd62f01.s1 | 323 | P | 183 | P | NC | −1.8 | −0.26 |
| | | 404F10 on chromo- | Soares | | | | | | | |
| | | some 1q23.1-24.1, | fetal heart | | | | | | | |
| | | CD48 hom | NbHH19W | | | | | | | |
| | | | Homo | | | | | | | |
| | | | sapiens | | | | | | | |
| | | | cDNA | | | | | | | |
| | | | clone | | | | | | | |
| | | | 345241 3′ | | | | | | | |
| | | | similar to | | | | | | | |
| | | | gb: M81181 | | | | | | | |
| | | | SODI- | | | | | | | |
| | | | UM/POTAS- | | | | | | | |
| | | | SIUM- | | | | | | | |
| | | | TRANS- | | | | | | | |
| | | | PORTING | | | | | | | |
| | | | ATPASE | | | | | | | |
| | | | BETA-2 | | | | | | | |
| | | | (HU- | | | | | | | |
| | | | MAN); con- | | | | | | | |
| | | | tains Alu | | | | | | | |
| | | | repetitive | | | | | | | |
| | | | element;. | | | | | | | |
| RC_AA255903_at | PPAAP | CD39L4; CD39-like 4; | zs28d05.s1 | 840 | P | 260 | P | NC | −3.4 | −2.13 |
| | | chrom 14 | NCI_CGAP_{—} | | | | | | | |
| | | | GCB1 | | | | | | | |
| | | | Homo | | | | | | | |
| | | | sapiens | | | | | | | |
| | | | cDNA | | | | | | | |
| | | | clone | | | | | | | |
| | | | IMAGE: | | | | | | | |
| | | | 686505 3′. | | | | | | | |

PPPPP; Inc in Duke BC
Avg Diff N >= 300; Fold change N to BC >= 5x

| | | | | | | | | | | |
|---|---|---|---|---|---|---|---|---|---|---|
| J03464_s_at | PPPPP | collagen alpha-2 type | Human | 352 | P | 1494 | P | I | 4.2 | 4.09 |
| | | I; mRNA COL2A1 | collagen | | | | | | | |
| | | | alpha-2 | | | | | | | |
| | | | type I | | | | | | | |
| | | | mRNA, | | | | | | | |
| | | | complete | | | | | | | |
| | | | cds, clone | | | | | | | |
| | | | pHCOL2A1 | | | | | | | |
| RC_N22015_at | PPPPP | unnamed protein | yw31h10.s1 | 638 | P | 3335 | P | I | 5.2 | 8.09 |
| | | product, chrom 17 | Homo | | | | | | | |
| | | | sapiens | | | | | | | |
| | | | CDNA | | | | | | | |
| | | | clone | | | | | | | |
| | | | 253891 3′. | | | | | | | |
| RC_AA055896_at | PPPPP | chrom 9 | zf20e03.s1 | 590 | P | 1835 | P | I | 2.9 | 2.15 |
| | | | Soares | | | | | | | |
| | | | fetal heart | | | | | | | |
| | | | NbHH19W | | | | | | | |
| | | | Homo | | | | | | | |
| | | | sapiens | | | | | | | |
| | | | cDNA | | | | | | | |
| | | | clone | | | | | | | |
| | | | 377500 3′. | | | | | | | |
| RC_AA121315_at | PPPPP | KIAA1077 protein; | zk91g08.s1 | 429 | P | 1764 | P | I | 4.9 | 5.57 |
| | | hypothetical protein, | Soares | | | | | | | |
| | | chrom 8 | pregnant | | | | | | | |
| | | | uterus | | | | | | | |
| | | | NbHPU | | | | | | | |
| | | | Homo | | | | | | | |
| | | | sapiens | | | | | | | |
| | | | cDNA | | | | | | | |
| | | | clone | | | | | | | |
| | | | 490238 3′. | | | | | | | |
| RC_AA122386_at | PPPPP | procollagen alpha | zn36f10.s1 | 466 | P | 1403 | P | I | 3 | 2.15 |
| | | 2(V); pro-alpha | Stratagene | | | | | | | |
| | | (V)collagen (AA 1099), | endothelial | | | | | | | |
| | | chrom 2 | cell | | | | | | | |
| | | | 937223 | | | | | | | |
| | | | Homo | | | | | | | |
| | | | sapiens | | | | | | | |
| | | | cDNA | | | | | | | |
| | | | clone | | | | | | | |
| | | | 549547 3′. | | | | | | | |

PPPPP; DEC in Duke BC
Avg Diff N >= 500; Fold change N to BC >= 5x

| | | | | | | | | | | |
|---|---|---|---|---|---|---|---|---|---|---|
| C02386_s_at | PPPPP | | HUMGS00 | 3429 | P | 1667 | P | D | −2.1 | −1.35 |
| | | | 10652, | | | | | | | |
| | | | Human | | | | | | | |
| | | | Gene | | | | | | | |
| | | | Signature, | | | | | | | |
| | | | 3′-directed | | | | | | | |
| | | | cDNA | | | | | | | |
| | | | sequence. | | | | | | | |
| M12272_s_at | PPPPP | alcohol dehydrogen- | Homo | 3368 | P | 904 | P | D | −3.4 | −4.07 |
| | | ase class I gamma | sapiens | | | | | | | |
| | | subunit (ADH3) | alcohol | | | | | | | |
| | | | dehydro- | | | | | | | |
| | | | genase | | | | | | | |
| | | | class I | | | | | | | |
| | | | gamma | | | | | | | |
| | | | subunit | | | | | | | |
| | | | (ADH3) | | | | | | | |
| | | | mRNA, | | | | | | | |
| | | | complete | | | | | | | |
| | | | cds | | | | | | | |
| M12759_at | PPPPP | Ig J chain gene | Human Ig | 2628 | P | 686 | P | D | −3.2 | −3.14 |
| | | | J chain | | | | | | | |
| | | | gene | | | | | | | |
| M83670_s_at | PPPPP | carbonic anhydrase | Human | 2138 | P | 432 | P | D | −5.1 | −6 |
| | | IV | carbonic | | | | | | | |
| | | | anhydrase | | | | | | | |
| | | | IV mRNA, | | | | | | | |
| | | | complete | | | | | | | |
| | | | cds | | | | | | | |
| N91087_at | PPPPP | dJ991C6.1 (novel | za18b11.r1 | 923 | P | 217 | P | D | −4.4 | −3.43 |
| | | protein similar to C. | Soares | | | | | | | |
| | | elegans F55A12.9 | fetal liver | | | | | | | |
| | | (Tr: P91086)); un- | spleen | | | | | | | |
| | | named protein | 1NFLS | | | | | | | |
| | | product; chrom 6 | Homo | | | | | | | |
| | | | sapiens | | | | | | | |
| | | | cDNA | | | | | | | |
| | | | clone | | | | | | | |
| | | | 292893 5′. | | | | | | | |
| RC_H77597_f_at | PPPPP | unident. EST chrom /// | ys08a06.s1 | 5411 | P | 1118 | P | D | −4.4 | −8.23 |
| | | AFFY = similar metal- | Homo | | | | | | | |
| | | lothionein | sapiens | | | | | | | |
| | | | cDNA | | | | | | | |
| | | | clone | | | | | | | |
| | | | 214162 3′ | | | | | | | |
| | | | similar to | | | | | | | |
| | | | gb: X64177 | | | | | | | |
| | | | H. sapiens | | | | | | | |
| | | | mRNA for | | | | | | | |
| | | | metallothi- | | | | | | | |
| | | | onein | | | | | | | |
| | | | (HUMAN);. | | | | | | | |
| RC_N23665_s_at | PPPPP | L11708 ESTRADIOL | yw40b12.s1 | 1150 | P | 246 | P | D | −4.1 | −3.18 |
| | | 17 BETA- | Homo | | | | | | | |
| | | DEHYDROGENASE | sapiens | | | | | | | |
| | | 2, chrom 16 | cDNA | | | | | | | |
| | | | clone | | | | | | | |
| | | | 254687 3′ | | | | | | | |
| | | | similar to | | | | | | | |
| | | | gb: L11708 | | | | | | | |
| | | | ESTRA- | | | | | | | |
| | | | DIOL 17 | | | | | | | |
| | | | BETA- | | | | | | | |
| | | | DEHYDRO- | | | | | | | |
| | | | GENASE | | | | | | | |
| | | | 2 (HU- | | | | | | | |
| | | | MAN);. | | | | | | | |
| RC_N79237_at | PPPPP | unnamed protein | za63a11.s1 | 7342 | P | 2621 | P | D | −2.8 | −4.25 |
| | | product, hom to | Homo | | | | | | | |
| | | S49589 cortical gran- | sapiens | | | | | | | |
| | | ule lectin - African | cDNA | | | | | | | |
| | | clawed frog;. | clone | | | | | | | |
| | | | 297212 3′ | | | | | | | |
| | | | similar to | | | | | | | |
| | | | PIR: S49589 | | | | | | | |
| | | | S49589 | | | | | | | |
| | | | cortical | | | | | | | |
| | | | granule | | | | | | | |
| | | | lectin - | | | | | | | |
| | | | African | | | | | | | |
| | | | clawed | | | | | | | |
| | | | frog;. | | | | | | | |
| RC_N80129_f_at | PPPPP | MT-11 protein; metal- | za65a05.s1 | 3623 | P | 1009 | P | D | −3.6 | −4.83 |
| | | lothionein IX; metal- | Homo | | | | | | | |
| | | lothionein 1L, chrom | sapiens | | | | | | | |
| | | 16 & chrom 1 | cDNA | | | | | | | |
| | | | clone | | | | | | | |
| | | | 297392 3′ | | | | | | | |
| | | | similar to | | | | | | | |
| | | | gb: X76717 | | | | | | | |
| | | | H. sapiens | | | | | | | |
| | | | MT-1I | | | | | | | |
| | | | mRNA. | | | | | | | |
| | | | (HUMAN);. | | | | | | | |
| RC_T24011_at | PPPPP | — | seq2167 | 900 | P | 317 | P | D | −2.8 | −1.53 |
| | | | Homo | | | | | | | |
| | | | sapiens | | | | | | | |
| | | | cDNA | | | | | | | |
| | | | clone | | | | | | | |
| | | | 3HFLSK20- | | | | | | | |
| | | | 53 3′. | | | | | | | |
| RC_T90492_at | PPPPP | IGJ, P01591 IMMU- | ye15c08.s1 | 4498 | P | 1829 | P | D | −2.5 | −2.47 |
| | | NOGLOBULIN J;. | Homo | | | | | | | |
| | | chrom 4 | sapiens | | | | | | | |
| | | | cDNA | | | | | | | |
| | | | clone | | | | | | | |
| | | | 117806 3′ | | | | | | | |
| | | | similar to | | | | | | | |
| | | | SP: IGJ_{—} | | | | | | | |
| | | | HUMAN | | | | | | | |
| | | | P01591 | | | | | | | |
| | | | IMMU- | | | | | | | |
| | | | NOGLOB- | | | | | | | |
| | | | ULIN J;. | | | | | | | |
| RC_AA058357_s_{—} | PPPPP | singleton; no hom., | zl67e01.s1 | 5524 | P | 1757 | P | D | −3.1 | −4.66 |
| at | | NONSPECIFIC | Stratagene | | | | | | | |
| | | CROSSREACTING | colon | | | | | | | |
| | | ANTIGEN.;. | (#937204) | | | | | | | |
| | | | Homo | | | | | | | |
| | | | sapiens | | | | | | | |
| | | | cDNA | | | | | | | |
| | | | clone | | | | | | | |
| | | | 509688 3′ | | | | | | | |
| | | | similar to | | | | | | | |
| | | | TR: | | | | | | | |
| | | | G189087 | | | | | | | |
| | | | G189087 | | | | | | | |
| | | | NONSPE- | | | | | | | |
| | | | CIFIC | | | | | | | |
| | | | CROSS- | | | | | | | |
| | | | REAC- | | | | | | | |
| | | | TING | | | | | | | |
| | | | ANTIGEN.;. | | | | | | | |
| RC_AA133469_at | PPPPP | cytokeratin 20, chrom | zo13e11.s1 | 3504 | P | 1231 | P | D | −2.4 | −1.87 |
| | | 17 | Strata- | | | | | | | |
| | | | gene colon | | | | | | | |
| | | | (#937204) | | | | | | | |
| | | | Homo | | | | | | | |
| | | | sapiens | | | | | | | |
| | | | cDNA | | | | | | | |
| | | | clone | | | | | | | |
| | | | 586796 3′. | | | | | | | |
| RC_AA151674_at | PPPPP | carbonic anhydrase | zo29e02.s1 | 1820 | P | 357 | P | D | −5.1 | −5.9 |
| | | XII precursor, chrom | Strata- | | | | | | | |
| | | 15 | gene colon | | | | | | | |
| | | | (#937204) | | | | | | | |
| | | | Homo | | | | | | | |
| | | | sapiens | | | | | | | |
| | | | cDNA | | | | | | | |
| | | | clone | | | | | | | |
| | | | 588314 3′. | | | | | | | |
| RC_AA405715_at | PPPPP | unknown | zu66d01.s1 | 4188 | P | 1080 | P | D | −3.9 | −5.93 |
| | | | Soares | | | | | | | |
| | | | testis NHT | | | | | | | |
| | | | Homo | | | | | | | |
| | | | sapiens | | | | | | | |
| | | | cDNA | | | | | | | |
| | | | clone | | | | | | | |
| | | | 742945 3′. | | | | | | | |
| RC_AA428410_at | PPPPP | KIAA0828 protein, | zw57g04.s1 | 964 | P | 277 | P | D | −3 | −1.72 |
| | | chrom 7 | Soares | | | | | | | |
| | | | total fetus | | | | | | | |
| | | | Nb2HF89w | | | | | | | |
| | | | Homo | | | | | | | |
| | | | sapiens | | | | | | | |
| | | | cDNA | | | | | | | |
| | | | clone | | | | | | | |
| | | | 774198 3′ | | | | | | | |
| | | | similar to | | | | | | | |
| | | | contains | | | | | | | |
| | | | Alu repeti- | | | | | | | |
| | | | tive ele- | | | | | | | |
| | | | ment; con- | | | | | | | |
| | | | tains ele- | | | | | | | |
| | | | ment | | | | | | | |
| | | | PTR7 | | | | | | | |
| | | | repetitive | | | | | | | |
| | | | element;. | | | | | | | |
| RC_AA429253_at | PPPPP | KIAA0803 protein; | zv50c08.s1 | 555 | P | 279 | P | D | −2 | −0.49 |
| | | Centrosome- and | Soares | | | | | | | |
| | | Golgi-localized PKN- | ovary | | | | | | | |
| | | associated protein | tumor | | | | | | | |
| | | (CG-NAP); AKAP450, | NbHOT | | | | | | | |
| | | A-kinase anchoring | Homo | | | | | | | |
| | | protein AKAP350, | sapiens | | | | | | | |
| | | chrom 7 | cDNA | | | | | | | |
| | | | clone | | | | | | | |
| | | | 757070 3′. | | | | | | | |
| RC_AA487468_at | PPPPP | chrom 7 | ab23b07.s1 | 820 | P | 263 | P | D | −2.6 | −1.13 |
| | | | Strata- | | | | | | | |
| | | | gene lung | | | | | | | |
| | | | (#937210) | | | | | | | |
| | | | Homo | | | | | | | |
| | | | sapiens | | | | | | | |
| | | | cDNA | | | | | | | |
| | | | clone | | | | | | | |
| | | | 841621 3′. | | | | | | | |
| RC_AA621680_at | PPPPP | zinc finger transcrip- | af48e09.s1 | 1201 | P | 295 | P | D | −4.1 | −3.43 |
| | | tion factor GKLF; EZF; | Soares | | | | | | | |
| | | hEZF; endothelial | total fetus | | | | | | | |
| | | Kruppel-like zinc finger | Nb2HF89w | | | | | | | |
| | | protein, transforming | Homo | | | | | | | |
| | | oncogene | sapiens | | | | | | | |
| | | | cDNA | | | | | | | |
| | | | clone | | | | | | | |
| | | | 1034920 | | | | | | | |
| | | | 3′. | | | | | | | |
| Y09616_at | PPPPP | putative carboxyles- | H. sapiens | 1776 | P | 605 | P | D | −3 | −2.48 |
| | | terase | mRNA for | | | | | | | |
| | | | putative | | | | | | | |
| | | | carboxyl- | | | | | | | |
| | | | esterase | | | | | | | |
| AA171913_at | PPPPP | carbonic anhydrase | zo95d05.r1 | 1117 | P | 148 | P | D | −7.2 | −6.85 |
| | | XII (CA12) chrom 15 | Strata- | | | | | | | |
| | | | gene | | | | | | | |
| | | | ovarian | | | | | | | |
| | | | cancer | | | | | | | |
| | | | (#937219) | | | | | | | |
| | | | Homo | | | | | | | |
| | | | sapiens | | | | | | | |
| | | | cDNA | | | | | | | |
| | | | clone | | | | | | | |
| | | | 594633 5′. | | | | | | | |
| AA253330_s_at | PPPPP | hypothetical protein; | zr72g02.r1 | 2590 | P | 740 | P | D | −3.5 | −3.91 |
| | | unnamed protein | Soares | | | | | | | |
| | | product, chrom 15 | NhHMPu | | | | | | | |
| | | | S1 Homo | | | | | | | |
| | | | sapiens | | | | | | | |
| | | | cDNA | | | | | | | |
| | | | clone | | | | | | | |
| | | | 668978 5′. | | | | | | | |

| | | | | | | | | | | |
|---|---|---|---|---|---|---|---|---|---|---|
| | | unknown genes, data | | | Diff | Fold | Sort | | | Diff |
| | Tumor | found after blast | Avg | Abs | Call | Change | Score | Avg | Abs | Call |
| EST candidates | stages | search | Diff | Call | Bvs | Bvs | Bvs | Diff | Call | Cvs |
| Probe Set | NABCD | homologous to | B | B | N | N | N | C | C | N |

LOST from N to tumour (tumour suppressor)

| | | | | | | | | | | |
|---|---|---|---|---|---|---|---|---|---|---|
| RC_H04768_at | PAAAA | chrom 15 no homology | 104 | A | D | −10.2 | −9.57 | 133 | A | D |
| RC_Z39652_at | PAAAA | Y14593 APM-1 gene | −212 | A | D | ˜−22.9 | −18.17 | −269 | A | D |
| | | adipocyte-specific | | | | | | | | |
| | | secretory protein: | | | | | | | | |
| | | chrom 1q21.3-q23 | | | | | | | | |
| RC_H30270_at | PAAAA | chrom 18 PAAAA in | 287 | A | D | −7.1 | −6.36 | 240 | A | D |
| | | colon & bladder no | | | | | | | | |
| | | homology | | | | | | | | |
| RC_T47089_s_at | PAAAA | tenascin-X; tenascin- | 79 | A | D | −10.4 | −8.51 | 15 | A | D |
| | | X precursor; unidenti- | | | | | | | | |
| | | fied protein | | | | | | | | |
| RC_W31906_at | PAAAA | secretagogin; | 148 | A | D | −2.6 | −1.18 | 263 | A | D |
| | | dJ501N12.8 (putative | | | | | | | | |
| | | protein) chrom 6 | | | | | | | | |
| RC_AA279803_at | PAAAA | chrom 2 no homology | 217 | A | D | −3 | −1.4 | 91 | A | D |
| RC_R01646_at | PAAAA | chrom 13q32.1-33.3; | 92 | A | D | −10.1 | −8.86 | 67 | A | D |
| | | AL159152; homolo- | | | | | | | | |
| | | gy to mouse Pcbp1 - | | | | | | | | |
| | | poly(rC)-binding | | | | | | | | |
| | | protein 1 | | | | | | | | |
| RC_AA099820_at | PAAAA | BAC clone AC016778 | 17 | A | D | ˜−10.0 | −6.91 | 38 | A | D |
| AA319615_at | PAAAA | secretory carrier | −198 | A | D | ˜−39.6 | −17.52 | −103 | A | D |
| | | membrane protein; | | | | | | | | |
| | | secretory carrier | | | | | | | | |
| | | membrane protein 2; | | | | | | | | |
| | | chrom 15 | | | | | | | | |
| H07011_at | PAAAA | tetraspan NET-6 | 5 | A | D | ˜−14.9 | −6.63 | 122 | A | D |
| | | mRNA; transmem- | | | | | | | | |
| | | brane 4 superfamlly; | | | | | | | | |
| | | chrom 7 | | | | | | | | |
| RC_T68873_f_at | PPAAA | | 269 | A | D | −13.5 | −22.23 | 14 | A | D |
| RC_T40995_f_at | PPAAA | | −8 | A | D | ˜−73.5 | −38.45 | 533 | A | D |
| RC_H81070_f_at | PPAAA | | −228 | A | D | ˜−87.0 | −43.73 | −100 | A | D |
| RC_N30796_at | PPPAA | | 217 | P | D | −5 | −4.48 | 345 | A | D |
| RC_W37778_f_at | PPAAA | | 174 | A | D | −6.5 | −6.21 | 237 | A | D |
| RC_R70212_s_at | PPAAA | | 84 | A | D | −8.5 | −6.7 | 109 | A | D |
| RC_AA426330_at | PPAAA | | 36 | A | D | ˜−19.0 | −11.89 | −227 | A | D |
| RC_N33927_s_at | PPAAA | | 151 | A | D | −4 | −2.33 | 222 | A | D |
| RC_T90190_s_at | PPAAA | | 33 | A | D | −14.8 | −8.64 | 56 | A | D |
| RC_AA447145_at | PPPAA | | 64 | P | D | −5.9 | −3.21 | 60 | A | D |
| RC_H75860_at | PPAAA | | 13 | A | D | ˜−11.1 | −6.17 | 5 | A | D |
| RC_T71132_s_at | PPAAA | | 54 | A | D | −6.4 | −3.4 | 73 | A | D |

GAINED from N to tumour (oncogene)

| | | | | | | | | | | |
|---|---|---|---|---|---|---|---|---|---|---|
| RC_AA609013_s_{—} | APPPP | microsomal dipepti- | 3117 | P | I | 6.7 | 10.33 | 3163 | P | I |
| at | | dase (also on 6.8 k); | | | | | | | | |
| | | chrom 16 | | | | | | | | |
| RC_AA232508_at | APPPP | CGI-89 protein; un- | 2138 | P | I | 4.1 | 4.71 | 1942 | P | I |
| | | named protein product; | | | | | | | | |
| | | hypothetical protein | | | | | | | | |
| RC_AA428964_at | APPPP | serine protease-like | 1894 | P | I | 5.2 | 6.55 | 969 | P | I |
| | | protease; serine pro- | | | | | | | | |
| | | tease homolog = NES1; | | | | | | | | |
| | | normal epithelial cell- | | | | | | | | |
| | | specific 1 | | | | | | | | |
| RC_T52813_s_at | APPPP | dJ28O10.2 (G0S2 | 1827 | P | I | 8.4 | 9.84 | 1597 | P | I |
| | | (PUTATIVE LYM- | | | | | | | | |
| | | PHOCYTE G0/G1 | | | | | | | | |
| | | SWITCH PROTEIN 2; | | | | | | | | |
| | | chrom 1 | | | | | | | | |
| RC_AA075642_at | APPPP | gp-340 variant protein; | 893 | P | I | 9 | 7.83 | 5271 | P | I |
| | | DMBT1/8kb.2 protein | | | | | | | | |
| RC_AA007218_at | APPPP | chrom 13 no homology | 878 | P | I | 5.6 | 4.65 | 884 | P | I |
| RC_N33920_at | APPPP | ubiquitin-like protein | 811 | P | I | ˜27.4 | 17.37 | 425 | P | I |
| | | FAT10; diubiquitin; | | | | | | | | |
| | | dJ271M21.6 (Diubiqui- | | | | | | | | |
| | | tin); chrom 6 | | | | | | | | |
| RC_N71781_at | APPPP | KIAA1199 protein, | 775 | P | I | ˜22.0 | 13.85 | 305 | P | I |
| | | chrom 15 | | | | | | | | |
| RC_R67275_s_at | APPPP | alpha-1 (type XI) | 699 | P | I | ˜17.6 | 10.87 | 468 | P | I |
| | | collagen precursor; | | | | | | | | |
| | | collagen, type XI, | | | | | | | | |
| | | alpha 1; collagen type | | | | | | | | |
| | | XI alpha-1 isoform A; | | | | | | | | |
| | | chrom 1 | | | | | | | | |
| RC_W80763_at | APPPP | hypothetical protein; | 658 | P | I | 4.2 | 2.67 | 789 | P | I |
| | | chrom 17 | | | | | | | | |
| RC_AA443793_at | APPPP | chrom 7p22 | 594 | P | I | 3.6 | 2.27 | 847 | P | I |
| | | AC006028 BAC clone | | | | | | | | |
| RC_AA034499_s_{—} | APPPP | ZNF198 protein; zinc | 594 | P | I | 4.7 | 2.98 | 536 | P | I |
| at | | finger protein; FIM | | | | | | | | |
| | | protein; Cys-rich pro- | | | | | | | | |
| | | tein; zinc finger protein | | | | | | | | |
| | | 198; chrom 13 | | | | | | | | |
| RC_AA035482_at | APPPP | chrom 5; AK022505 | 506 | P | I | 1.9 | 0.39 | 572 | P | I |
| | | clone; CalcineurinB | | | | | | | | |
| | | (weakly similar) | | | | | | | | |
| RC_AA024482_at | APPPP | hypothetical protein; | 445 | P | I | ˜26.6 | 12.37 | 360 | P | I |
| | | unnamed protein | | | | | | | | |
| | | product; chrom 17 | | | | | | | | |
| RC_H93021_at | APPPP | chrom 2; XM_004890 | 436 | P | I | 3.7 | 2.12 | 384 | P | I |
| | | peptidylprolyl isomer- | | | | | | | | |
| | | ase A (cyclophilin A) | | | | | | | | |
| RC_AA427737_at | APPPP | no homology | 419 | P | I | 2.9 | 1.07 | 515 | P | I |
| RC_AA417078_at | APPPP | chrom 7q31; | 411 | P | I | 3.3 | 1.34 | 462 | P | I |
| | | AF017104 clone | | | | | | | | |
| M29873_s_at | APPPP | cytochrome P450-IIB | 372 | P | I | 5.2 | 2.7 | 760 | P | I |
| | | (hIIB3); 19q13.1- | | | | | | | | |
| | | q13.2 | | | | | | | | |
| RC_H27498_f_at | AAPPP | | 6246 | P | I | 2.4 | 2.87 | 9295 | P | I |
| RC_T92363_s_at | AAPPP | | 730 | P | I | 10.7 | 8.3 | 238 | P | I |
| RC_N89910_at | AAAPP | | 149 | A | NC | 1.1 | 0.01 | 301 | P | I |
| RC_W60516_at | AAAPP | | 328 | A | NC | 1 | 0 | 437 | P | I |
| RC_AA219699_at | AAAPP | | 109 | A | MI | ˜2.6 | 0.35 | 114 | P | I |
| RC_AA449450_at | AAAPP | | 526 | A | NC | 2.2 | 0.65 | 411 | P | I |

A classifiers
Gained in Duke A and Inc; NC in other Dukes;
Avg Diff in A > 300

| | | | | | | | | | | |
|---|---|---|---|---|---|---|---|---|---|---|
| RC_AA599199_at | APAAA | ALU seq. | 284 | A | NC | ˜8.8 | 4.36 | 176 | A | NC |
| RC_R12694_at | APAAA | unnamed protein | 200 | A | NC | 1.9 | 0.27 | 208 | A | NC |
| | | product BAA91641, | | | | | | | | |
| | | chrom 10 | | | | | | | | |
| RC_H91325_s_at | APAAA | aldolase B; aldolase B | 157 | A | NC | 1.7 | 0.16 | 71 | A | NC |
| | | (aa 1-364); chrom 9 | | | | | | | | |
| RC_N51709_at | APAAA | chrom X | 378 | A | NC | −1.2 | −0.05 | 473 | A | NC |
| RC_N72610_at | APAAA | — | 338 | A | NC | 1.5 | 0.13 | 618 | A | NC |

Lost in Duke A and Dec; NC in other Dukes
Avg Diff in N > 300

| | | | | | | | | | | |
|---|---|---|---|---|---|---|---|---|---|---|
| RC_N32411_f_at | PAPPP | Myc-associated zinc- | 3491 | P | NC | −1.2 | −0.11 | 4943 | P | NC |
| | | finger protein of hu- | | | | | | | | |
| | | man islet; chrom 16 | | | | | | | | |
| RC_AA243858_at | PAPPP | KIAA0882 protein | 548 | P | NC | 1.4 | 0.13 | 334 | P | NC |
| RC_AA486283_at | PAPPP | ras-like protein; ras- | 3548 | P | NC | −1.4 | −0.33 | 5135 | P | NC |
| | | related C3 botulinum | | | | | | | | |
| | | toxin substrate; | | | | | | | | |
| | | dJ20J23 | | | | | | | | |
| RC_AA490930_at | PAPPP | chrom 18; KIAA1468 | 214 | P | NC | −2.6 | −0.97 | 225 | P | NC |
| | | protein | | | | | | | | |

PPPPP; Inc in Duke A; NC in other Dukes
Avg Diff N >= 200; Fold change N to A >= 3x

| | | | | | | | | | | |
|---|---|---|---|---|---|---|---|---|---|---|
| RC_N69263_at | PPPPP | chrom 10; AK026414 | 909 | P | NC | 2.5 | 1.12 | 681 | P | NC |
| | | clone (only 108 nt | | | | | | | | |
| | | hom) | | | | | | | | |
| RC_T15817_f_at | PPPPP | iNOS, inducible nitric | 606 | P | NC | 1.3 | 0.06 | 865 | P | NC |
| | | oxide synthase | | | | | | | | |

PPPPP; Dec in Duke A; NC in other Dukes
Avg Diff N >= 200; Fold change N to A >= 3x

| | | | | | | | | | | |
|---|---|---|---|---|---|---|---|---|---|---|
| RC_H54088_s_at | PPPPP | ribosomal protein L41 | 4157 | P | NC | −1.3 | −0.22 | 6623 | P | NC |
| RC_H59052_f_at | PPPPP | fungal sterol-C5- | 4077 | P | NC | −1.2 | −0.18 | 5764 | P | NC |
| | | desaturase homolog; | | | | | | | | |
| | | ORF; thymosin beta-4 | | | | | | | | |
| RC_R49198_s_at | PPPPP | — | 3144 | P | NC | −1.5 | −0.58 | 4785 | P | NC |
| RC_T73572_f_at | PPPPP | ferritin L-chain; L | 5148 | P | NC | −1.1 | −0.06 | 6119 | P | NC |
| | | apoferritin | | | | | | | | |
| RC_AA477483_at | PPPPP | no matching est | 3186 | P | NC | −1.4 | −0.37 | 4175 | P | NC |

B classifiers
Gained in Duke B and Inc; NC in other Dukes
Avg Diff in B > 300

| | | | | | | | | | | |
|---|---|---|---|---|---|---|---|---|---|---|
| RC_T67463_s_at | AAPAA | cathepsin O2; X; K | 713 | P | I | 5.4 | 3.97 | 226 | A | NC |
| RC_W94688_at | AAPAA | perilipin | 393 | P | I | 2.8 | 1 | 84 | A | NC |
| RC_AA126743_at | AAPAA | Z97200 PAC chrom | 734 | P | I | 1.8 | 0.43 | 98 | A | NC |
| | | 1q24; PMX1 homeo- | | | | | | | | |
| | | box gene | | | | | | | | |
| RC_AA236547_at | AAPAA | no homology | 402 | P | I | 1.8 | 0.3 | 285 | A | NC |
| RC_AA255567_at | AAPAA | angiopoietin-related | 408 | P | I | 3.4 | 1.5 | 207 | A | NC |
| | | protein-2; angiopoietin- | | | | | | | | |
| | | like 2 | | | | | | | | |
| RC_AA421256_at | AAPAA | — | 382 | P | I | 1.1 | 0.02 | 236 | A | NC |

Lost in Duke B and Dec; NC in other Dukes
Avg Diff in N > 300

| | | | | | | | | | | |
|---|---|---|---|---|---|---|---|---|---|---|
| RC_D59847_at | PPAPP | proSAAS; granin-like | 336 | A | D | −2.6 | −1.2 | 308 | P | NC |
| | | neuroendocrine pep- | | | | | | | | |
| | | tide precursor | | | | | | | | |
| RC_F05038_at | PPAPP | polyamine modulated | 972 | A | D | −1.9 | −0.84 | 1644 | P | NC |
| | | factor-1; polyamine | | | | | | | | |
| | | modulated factor 1 | | | | | | | | |
| RC_N41059_at | PPAPP | chrom 3 | 246 | A | D | −1.4 | −0.11 | 378 | P | NC |
| RC_T23460_at | PPAPP | chrom 3; IFNAR2 | 402 | A | D | −2 | −0.53 | 570 | P | NC |
| | | 21q22.11 | | | | | | | | |
| RC_W42789_at | PPAPP | chrom 8 AF268037 | 29 | A | D | ˜−5.6 | −2.73 | 129 | P | NC |
| | | C8ORF4 protein | | | | | | | | |
| | | (C8ORF4) chrom 8 | | | | | | | | |
| | | ORF | | | | | | | | |
| RC_AA460017_L_{—} | PPAPP | BAC clone chrom 16 | 725 | A | D | −1.9 | −0.66 | 666 | P | NC |
| at | | | | | | | | | | |
| RC_AA482127_at | PPAPP | KIAA1142 protein | 138 | A | D | −2.6 | −0.78 | 211 | P | NC |
| RC_AA504806_at | PPAPP | chrom 2 AF052107 | 135 | A | D | −4.5 | −2.02 | 351 | P | NC |
| | | clone 23620 mRNA | | | | | | | | |
| | | sequence | | | | | | | | |

Inc in Duke B; NC in other Dukes
Avg Diff N >= 200; Fold change N to A >= 3x

| | | | | | | | | | | |
|---|---|---|---|---|---|---|---|---|---|---|
| RC_AA386386_s_{—} | PPPPP | — | 4776 | P | I | 3 | 3.89 | 1559 | P | NC |
| at | | | | | | | | | | |
| RC_AA452549_at | PPPPP | PR01659; hypotheti- | 1255 | P | I | 3.1 | 2.22 | 422 | P | NC |
| | | cal protein chrom 11 | | | | | | | | |

Dec in Duke B; NC in other dukes
Avg Diff N >= 200; Fold change N to B >= 3x

| | | | | | | | | | | |
|---|---|---|---|---|---|---|---|---|---|---|
| RC_T90037_at | PPPPP | unnamed protein | 204 | P | D | −4.6 | −3.72 | 395 | P | NC |
| | | product, chrom 4 | | | | | | | | |
| RC_AA432130_at | PPPPP | KIAA0867 protein, | 226 | P | D | −5.4 | −5.23 | 534 | P | NC |
| | | chrom 12 | | | | | | | | |

C classifiers
Gained in Duke C and Inc; NC in other Dukes
Avg Diff in C > 300

| | | | | | | | | | | |
|---|---|---|---|---|---|---|---|---|---|---|
| RC_D45556_at | AAAPA | chrom 15; AL390085 | 407 | A | NC | 1 | 0 | 536 | P | I |
| | | clone | | | | | | | | |
| RC_W86214_at | AAAPA | | 305 | A | NC | 2.2 | 0.39 | 409 | P | I |
| RC_AA039439_s_{—} | AAAPA | novel gene KIAA0134 | 320 | A | NC | 3.2 | 1.39 | 600 | P | I |
| at | | protein 19q13.3 | | | | | | | | |
| RC_AA128935_at | AAAPA | | 245 | A | NC | 1.5 | 0.12 | 416 | P | I |
| RC_AA134158_s_{—} | AAAPA | class I homeodomain; | 273 | A | NC | 5 | 2.21 | 988 | P | I |
| at | | homeobox protein, | | | | | | | | |
| | | chrom 7 | | | | | | | | |
| RC_AA232646_at | AAAPA | chrom 17, AF266756 | 601 | A | NC | 4.9 | 3.21 | 608 | P | I |
| | | sphingosine kinase | | | | | | | | |
| | | (SPHK1 | | | | | | | | |
| RC_AA401184_at | AAAPA | no homology | 224 | A | NC | ˜−2.2 | −0.1 | 502 | P | I |
| RC_AA436840_at | AAAPA | | 367 | A | NC | 1.4 | 0.1 | 458 | P | I |
| RC_AA488655_at | AAAPA | | 257 | A | NC | ˜9.5 | 4.43 | 360 | P | I |

L st in Duke C and Dec; NC in other Dukes
Avg Diff in N > 300

| | | | | | | | | | | |
|---|---|---|---|---|---|---|---|---|---|---|
| RC_N30231_at | PPPAP | Lsm4 protein; U6 | 1161 | P | NC | −1.3 | −0.13 | −978 | A | D |
| | | snRNA-associated | | | | | | | | |
| | | Sm-like protein LSm4; | | | | | | | | |
| | | glycine-rich protein | | | | | | | | |
| RC_W73790_f_at | PPPAP | immunoglobulin- | 728 | P | NC | −4.2 | −5.76 | 759 | A | D |
| | | related protein 14.1; | | | | | | | | |
| | | lambda L-chain C | | | | | | | | |
| | | region; omega protein, | | | | | | | | |
| | | chrom 22 | | | | | | | | |
| RC_AA412184_at | PPPAP | chrom 1p36; d89060 | 488 | P | NC | −1.5 | −0.18 | 437 | A | D |
| | | dolichyl- | | | | | | | | |
| | | diphosphooligosaccha- | | | | | | | | |
| | | ride-protein glycosyl- | | | | | | | | |
| | | transferase | | | | | | | | |
| RC_AA521303_at | PPPAP | methionine adenosyl- | 479 | P | NC | −1.6 | −0.29 | 338 | A | D |
| | | transferase regulatory | | | | | | | | |
| | | beta subunit; dTDP-4- | | | | | | | | |
| | | keto-6-deoxy-D- | | | | | | | | |
| | | glucose 4-reductase, | | | | | | | | |
| | | chrom 5 | | | | | | | | |

Inc in Duke C; NC in other Dukes
Avg Diff N >= 200; Fold change N to C >= 3x

| | | | | | | | | | | |
|---|---|---|---|---|---|---|---|---|---|---|
| RC_AA181902_at | PPPPP | AC007201 on chrom | 614 | P | NC | 1.5 | 0.21 | 1347 | P | I |
| | | 19 (only 80 nt hom) | | | | | | | | |

Dec in Duke C; NC in other Dukes
Avg Diff N >= 200; Fold change N to D >= 3x

| | | | | | | | | | | |
|---|---|---|---|---|---|---|---|---|---|---|
| RC_AA461174_at | PPPPP | 8p21.3-p22 AB020860 | 169 | P | NC | −1.7 | −0.23 | 84 | P | D |
| | | anti-oncogene | | | | | | | | |
| AA393432_s_at | PPPPP | chrom 2, Unknown; | 159 | P | NC | −2 | −0.38 | 97 | P | D |
| | | unnamed protein | | | | | | | | |
| | | product AAD20029 | | | | | | | | |

D classifier
Gained in Duke D and Inc; NC in other Dukes
Avg Diff in D > 300

| | | | | | | | | | | |
|---|---|---|---|---|---|---|---|---|---|---|
| RC_N91920_at | AAAAP | chrom 16p12-p11.2; | 123 | A | NC | ˜6.6 | 1.88 | −70 | A | NC |
| | | XN_007994 retinobla- | | | | | | | | |
| | | stoma binding protein | | | | | | | | |
| RC_AA621601_at | AAAAP | chrom 17 XM_009868 | 185 | A | NC | ˜1.3 | 0.01 | 18 | A | NC |
| | | RAB36 ARS oncogene | | | | | | | | |
| | | family | | | | | | | | |

Lost in Duke D and Dec; NC in other Dukes
Avg Diff in N > 500

| | | | | | | | | | | |
|---|---|---|---|---|---|---|---|---|---|---|
| RC_R72886_s_at | PPPPA | KIAA0422; adenylyl | 1052 | P | NC | −1.7 | −0.53 | 1528 | P | NC |
| | | cyclase type VI, chrom | | | | | | | | |
| | | 12 | | | | | | | | |
| RC_AA026030_at | PPPPA | chrom 1 | 1074 | P | NC | −1.4 | −0.19 | 1263 | P | NC |
| RC_Z39006_at | PPPPA | hypothetical protein, | 824 | P | NC | −1.5 | −0.28 | 876 | P | NC |
| | | chrom 17 | | | | | | | | |
| RC_AA435908_at | PPPPA | chrom 19; ac011491 | 673 | P | NC | −1.2 | −0.04 | 394 | P | NC |
| | | clone and 20 nt hom. | | | | | | | | |
| | | RAB2, RAS oncogene | | | | | | | | |
| | | family | | | | | | | | |
| RC_AA057829_s_{—} | PPPPA | growth-arrest-specific | 1572 | P | NC | 1.6 | 0.44 | 936 | P | NC |
| at | | protein; growth arrest- | | | | | | | | |
| | | specific 6; AXL stimu- | | | | | | | | |
| | | latory factor, chrom 13 | | | | | | | | |
| RC_R72087_at | PPPPA | chrom 5 EST; hom to | 726 | P | NC | −1.3 | −0.09 | 971 | P | NC |
| | | chrom 20 AL356652 | | | | | | | | |
| | | clone | | | | | | | | |
| RC_H04242_at | PPPPA | ras related protein | 365 | P | NC | −1.4 | −0.1 | 475 | P | NC |
| | | Rab5b; RAB5B, mem- | | | | | | | | |
| | | ber RAS oncogene | | | | | | | | |
| | | family | | | | | | | | |
| RC_R97304_f_at | PPPPA | HLA-drb5; cell surface | 556 | P | NC | −1.5 | −0.25 | 680 | P | NC |
| | | glycoprotein; MHC | | | | | | | | |
| | | HLA-DR-beta chain | | | | | | | | |
| | | precursor chrom 6 | | | | | | | | |
| RC_N48609_at | PPPPA | chrom 11; AC004584 | 884 | P | NC | 1.1 | 0.04 | 834 | P | NC |
| | | chrom 17 | | | | | | | | |
| RC_W86850_f_at | PPPPA | chrom 22 ? X96924 | −63 | P | NC | ˜−23.9 | −13.75 | 474 | P | NC |
| | | mitochondrial citrate | | | | | | | | |
| | | tranbsport region | | | | | | | | |
| RC_AA130603_at | PPPPA | ak024908 clone | 681 | P | NC | 1.1 | 0.01 | 598 | P | NC |
| RC_AA479610_at | PPPPA | singleton ak025344 | 618 | P | NC | −1 | 0 | 726 | P | NC |
| | | clone | | | | | | | | |
| RC_AA490593_l_at | PPPPA | chrom 17 ? Synapto- | 610 | P | NC | −1.1 | −0.02 | 518 | P | NC |
| | | brevin2 (VAMP2) | | | | | | | | |
| | | AF135372 | | | | | | | | |
| RC_AA054321_s_{—} | PPPPA | 6p21 HLA class i | 346 | P | NC | −1.5 | −0.15 | 441 | P | NC |
| at | | region; AC004202 | | | | | | | | |
| | | clone | | | | | | | | |

Inc in Duke D; NC in other Dukes
Avg Diff N >= 200; Fold change N to A >= 3x

| | | | | | | | | | | |
|---|---|---|---|---|---|---|---|---|---|---|
| D79052_s_at | PPPPP | Sec61 gamma | 856 | P | NC | −1 | 0 | 868 | P | NC |
| RC_T40439_s_at | PPPPP | U2 small nuclear | 934 | P | NC | 1.2 | 0.07 | 1125 | P | NC |
| | | ribonucleoprotein B″; | | | | | | | | |
| | | dJ705D16.1 | | | | | | | | |
| RC_AA251829_at | PPPPP | CGI-29 protein | 440 | P | NC | 2.2 | 0.57 | 366 | P | NC |

Dec in Duke D; NC in other Dukes
Avg Diff N >= 500; Fold change N to D >= −5x

| | | | | | | | | | | |
|---|---|---|---|---|---|---|---|---|---|---|
| RC_D60328_at | PPPPP | chrom 6, unknown; | 738 | P | NC | −1.8 | −0.58 | 722 | P | NC |
| | | ring finger protein 5 | | | | | | | | |
| RC_H96850_at | PPPPP | oligosaccharyltransfer- | 4062 | P | NC | −1.2 | −0.13 | 3685 | P | NC |
| | | ase d89060 1p36.1 | | | | | | | | |
| | | (also C-class) | | | | | | | | |
| RC_AA127444_at | PPPPP | chrom 1 no homology | 364 | P | NC | −1.7 | −0.33 | 427 | P | NC |
| RC_AA242824_at | PPPPP | chrom 11; ac005233 | 246 | P | NC | −1.3 | −0.07 | 144 | P | NC |
| | | PAC clone chrom 22 | | | | | | | | |
| AA405775_s_at | PPPPP | similar to CAA16821 | 804 | P | NC | −1.3 | −0.11 | 791 | P | NC |
| | | (PID: g3255952) | | | | | | | | |

AB classifiers
Gained in Duke AB and Inc; NC in other Dukes
Avg Diff in AB >= 300

| | | | | | | | | | | |
|---|---|---|---|---|---|---|---|---|---|---|
| RC_AA136269_at | APPAA | HSPC314, chrom 12 | 266 | P | I | 2.9 | 1.25 | 85 | A | NC |

Lost in Duke AB and Dec; NC in other Dukes
Avg Diff in N > 300

| | | | | | | | | | | |
|---|---|---|---|---|---|---|---|---|---|---|
| RC_T40895_at | PAAPP | protein tyrosine phos- | 41 | A | D | ˜−12.7 | −9.62 | 720 | P | D |
| | | phatase PTPCAAX1; | | | | | | | | |
| | | protein tyrosine phos- | | | | | | | | |
| | | phatase hPRL-1N; iva- | | | | | | | | |
| | | 1, chrom 6 | | | | | | | | |
| RC_AA424400_at | PAAPP | chrom 14 | 120 | A | D | −2.9 | −0.97 | 180 | P | NC |

PPPPP; Inc in Duke AB
Avg Diff AB >= 300; Fold change N to AB >= 5x

| | | | | | | | | | | |
|---|---|---|---|---|---|---|---|---|---|---|
| RC_N22015_at | PPPPP | unnamed protein | 3608 | P | I | 5.3 | 8.6 | 2944 | P | I |
| | | product, chrom 17; | | | | | | | | |
| | | BC class. | | | | | | | | |
| RC_N50971_at | PPPPP | — | 581 | P | I | 6.8 | 4.78 | 761 | P | I |
| RC_AA194833_at | PPPPP | senescence- | 1316 | P | I | 7.6 | 8.35 | 328 | P | I |
| | | associated epithelial | | | | | | | | |
| | | membrane protein; | | | | | | | | |
| | | Claudin-1, chrom 3 | | | | | | | | |
| RC_AA393803_at | PPPPP | chrom 6 | 503 | P | I | 9.2 | 6.04 | 359 | P | I |

BC classifiers
Gained in Duke BC and Inc; NC in other Dukes
Avg Diff in BC > 300

| | | | | | | | | | | |
|---|---|---|---|---|---|---|---|---|---|---|
| RC_AA148923_at | AAPPA | DEPP; decidual pro- | 731 | P | I | 4 | 2.7 | 507 | P | I |
| | | tein induced by proges- | | | | | | | | |
| | | terone; hypothetical | | | | | | | | |
| | | protein, chrom 10 | | | | | | | | |

Lost in Duke BC and Dec
Avg Diff in N > 300

| | | | | | | | | | | |
|---|---|---|---|---|---|---|---|---|---|---|
| RC_N91919_s_at | PPAAP | mineralocorticoid | 16 | A | D | ˜−13.4 | −7.34 | 39 | A | D |
| | | receptor; mineralocor- | | | | | | | | |
| | | ticoid receptor (al- | | | | | | | | |
| | | dosterone receptor); | | | | | | | | |
| | | chrom 4 | | | | | | | | |
| RC_T99196_s_at | PPAAP | chrom 5 AF010235 | 143 | A | D | −3.7 | −1.94 | 197 | A | D |
| | | Homo sapiens mRNA | | | | | | | | |
| | | from chromosome | | | | | | | | |
| | | 5q31-33 region | | | | | | | | |
| RC_W72366_at | PPAAP | chrom 1 clone RP11- | 69 | A | D | −4.7 | −2.2 | 115 | A | D |
| | | 404F10 on chromo- | | | | | | | | |
| | | some 1q23.1-24.1, | | | | | | | | |
| | | CD48 hom | | | | | | | | |
| RC_AA255903_at | PPAAP | CD39L4; CD39-like 4; | −36 | A | D | ˜−25.4 | −15.91 | 153 | A | D |
| | | chrom 14 | | | | | | | | |

PPPPP; Inc in Duke BC
Avg Diff N >= 300; Fold change N to BC >= 5x

| | | | | | | | | | | |
|---|---|---|---|---|---|---|---|---|---|---|
| J03464_s_at | PPPPP | collagen alpha-2 type | 3072 | P | I | 8.7 | 14.19 | 2027 | P | I |
| | | I mRNA COL2A1 | | | | | | | | |
| RC_N22015_at | PPPPP | unnamed protein | 3608 | P | I | 5.3 | 8.6 | 2944 | P | I |
| | | product, chrom 17 | | | | | | | | |
| RC_AA055896_at | PPPPP | chrom 9 | 3885 | P | I | 6.4 | 11.26 | 2467 | P | I |
| RC_AA121315_at | PPPPP | KIAA1077 protein; | 3493 | P | I | 9.8 | 16.81 | 1724 | P | I |
| | | hypothetical protein, | | | | | | | | |
| | | chrom 8 | | | | | | | | |
| RC_AA122386_at | PPPPP | procollagen alpha | 2356 | P | I | 5.1 | 6.63 | 1725 | P | I |
| | | 2(V); pro-alpha | | | | | | | | |
| | | (V)collagen (AA 1099), | | | | | | | | |
| | | chrom 2 | | | | | | | | |

PPPPP; DEC in Duke BC
Avg Diff N >= 500; Fold change N to BC >= 5x

| | | | | | | | | | | |
|---|---|---|---|---|---|---|---|---|---|---|
| C02386_s_at | PPPPP | | 469 | P | D | −7.1 | −11.91 | 640 | P | D |
| M12272_s_at | PPPPP | alcohol dehydrogen- | 266 | P | D | −11.6 | −18.17 | 120 | P | D |
| | | ase class I gamma | | | | | | | | |
| | | subunit (ADH3) | | | | | | | | |
| M12759_at | PPPPP | Ig J chain gene | 239 | P | D | −8.6 | −11.42 | 107 | P | D |
| M83670_s_at | PPPPP | carbonic anhydrase | 249 | P | D | −9.6 | −12.13 | 342 | P | D |
| | | IV | | | | | | | | |
| N91087_at | PPPPP | dJ991C6.1 (novel | 173 | P | D | −5.3 | −4.45 | 167 | P | D |
| | | protein similar to C. | | | | | | | | |
| | | elegans F55A12.9 | | | | | | | | |
| | | (Tr: P91086)); un- | | | | | | | | |
| | | named protein | | | | | | | | |
| | | product; chrom 6 | | | | | | | | |
| RC_H77597_f_at | PPPPP | unident. EST chrom /// | 762 | P | D | −7.1 | −15.26 | 619 | P | D |
| | | AFFY = similar metal- | | | | | | | | |
| | | lothionein | | | | | | | | |
| RC_N23665_s_at | PPPPP | L11708 ESTRADIOL | 84 | P | D | −12 | −10.65 | 68 | P | D |
| | | 17 BETA- | | | | | | | | |
| | | DEHYDROGENASE | | | | | | | | |
| | | 2, chrom 16 | | | | | | | | |
| RC_N79237_at | PPPPP | unnamed protein | 237 | P | D | −29.6 | −48.42 | 458 | P | D |
| | | product, hom to | | | | | | | | |
| | | S49589 cortical gran- | | | | | | | | |
| | | ule lectin - African | | | | | | | | |
| | | clawed frog;. | | | | | | | | |
| RC_N80129_f_at | PPPPP | MT-11 protein; metal- | 323 | P | D | −11.2 | −19.21 | 516 | P | D |
| | | lothionein IX; metal- | | | | | | | | |
| | | lothionein 1L, chrom | | | | | | | | |
| | | 16 & chrom 1 | | | | | | | | |
| RC_T24011_at | PPPPP | — | 177 | P | D | −5.1 | −4.15 | 157 | P | D |
| RC_T90492_at | PPPPP | IGJ, P01591 IMMU- | 468 | P | D | −10.3 | −20.59 | 321 | P | D |
| | | NOGLOBULIN J;. | | | | | | | | |
| | | chrom 4 | | | | | | | | |
| RC_AA058357_s_{—} | PPPPP | singleton; no hom., | 232 | P | D | −20.3 | −32.79 | 460 | P | D |
| at | | NONSPECIFIC | | | | | | | | |
| | | CROSSREACTING | | | | | | | | |
| | | ANTIGEN.;. | | | | | | | | |
| RC_AA133469_at | PPPPP | cytokeratin 20, chrom | 453 | P | D | −6.5 | −10.18 | 375 | P | D |
| | | 17 | | | | | | | | |
| RC_AA151674_at | PPPPP | carbonic anhydrase | 188 | P | D | −8.8 | −10.51 | 116 | P | D |
| | | XII precursor, chrom | | | | | | | | |
| | | 15 | | | | | | | | |
| RC_AA405715_at | PPPPP | unknown | 542 | P | D | −7.7 | −14.69 | 521 | P | D |
| RC_AA428410_at | PPPPP | KIAA0828 protein, | 141 | P | D | −8.6 | −6.99 | 53 | P | D |
| | | chrom 7 | | | | | | | | |
| RC_AA429253_at | PPPPP | KIAA0803 protein; | 103 | P | D | −5.4 | −3.5 | 111 | P | D |
| | | Centrosome- and | | | | | | | | |
| | | Golgi-localized PKN- | | | | | | | | |
| | | associated protein | | | | | | | | |
| | | (CG-NAP); AKAP450, | | | | | | | | |
| | | A-kinase anchoring | | | | | | | | |
| | | protein AKAP350, | | | | | | | | |
| | | chrom 7 | | | | | | | | |
| RC_AA487468_at | PPPPP | chrom 7 | 106 | P | D | −6.5 | −4.92 | 95 | P | D |
| RC_AA621680_at | PPPPP | zinc finger transcrip- | 110 | P | D | −10.9 | −10.81 | 124 | P | D |
| | | tion factor GKLF; EZF; | | | | | | | | |
| | | hEZF; endothelial | | | | | | | | |
| | | Kruppel-like zinc finger | | | | | | | | |
| | | protein, transforming | | | | | | | | |
| | | oncogene | | | | | | | | |
| Y09616_at | PPPPP | putative carboxyles- | 299 | P | D | −6.1 | −7.47 | 260 | P | D |
| | | terase | | | | | | | | |
| AA171913_at | PPPPP | carbonic anhydrase | 78 | P | D | −13.6 | −11.99 | 85 | P | D |
| | | XII (CA12) chrom 15 | | | | | | | | |
| AA253330_s_at | PPPPP | hypothetical protein; | 448 | P | D | −6 | −8.89 | 420 | P | D |
| | | unnamed protein | | | | | | | | |
| | | product, chrom 15 | | | | | | | | |

| | | | | | | | | | |
|---|---|---|---|---|---|---|---|---|---|
| | | unknown genes, data | Fold | Sort | | | Diff | Fld | Sort |
| | Tumor | found after blast | Change | Score | Avg | Abs | Call | Change | Score |
| EST candidates | stages | search | Cvs | Cvs | Diff | Call | Dvs | Dvs | Dvs |
| Probe Set | NABCD | homologous to | N | N | D | D | N | N | N |

LOST from N to tumour (tumour suppressor)

| | | | | | | | | | |
|---|---|---|---|---|---|---|---|---|---|
| RC_H04768_at | PAAAA | chrom 15 no homology | −9.3 | −9.54 | 46 | A | D | ˜−7.8 | −0.22 |
| RC_Z39652_at | PAAAA | Y14593 APM-1 gene | ˜−21.5 | −16.51 | 16 | A | D | ˜−7.0 | −0.53 |
| | | adipocyte-specific | | | | | | | |
| | | secretory protein: | | | | | | | |
| | | chrom 1q21.3-q23 | | | | | | | |
| RC_H30270_at | PAAAA | chrom 18 PAAAA in | −8.1 | −7.28 | 114 | A | D | ˜−5.7 | −8.11 |
| | | colon & bladder no | | | | | | | |
| | | homology | | | | | | | |
| RC_T47089_s_at | PAAAA | tenascin-X; tenascin- | ˜−21.5 | −14.77 | −42 | A | D | ˜−21.7 | −4.93 |
| | | X precursor; unidenti- | | | | | | | |
| | | fied protein | | | | | | | |
| RC_W31906_at | PAAAA | secretagogin; | −2.8 | −1.34 | 147 | A | D | ˜−17.0 | −0.4 |
| | | dJ501N12.8 (putative | | | | | | | |
| | | protein) chrom 6 | | | | | | | |
| RC_AA279803_at | PAAAA | chrom 2 no homology | −4.3 | −2.73 | 161 | A | D | −11.1 | −12.8 |
| RC_R01646_at | PAAAA | chrom 13q32.1-33.3; | −13.8 | −11.32 | −37 | A | D | ˜−6.5 | −5.64 |
| | | AL159152; homolo- | | | | | | | |
| | | gy to mouse Pcbp1 - | | | | | | | |
| | | poly(rC)-binding | | | | | | | |
| | | protein 1 | | | | | | | |
| RC_AA099820_at | PAAAA | BAC clone AC016778 | ˜−9.8 | −6.76 | −5 | A | D | ˜−4.7 | −0.52 |
| AA319615_at | PAAAA | secretory carrier | ˜−34.8 | −15.82 | −225 | A | D | ˜−35.4 | −14.53 |
| | | membrane protein; | | | | | | | |
| | | secretory carrier | | | | | | | |
| | | membrane protein 2; | | | | | | | |
| | | chrom 15 | | | | | | | |
| H07011_at | PAAAA | tetraspan NET-6 | −2.6 | −0.79 | 193 | A | D | −1.7 | −9.87 |
| | | mRNA; transmem- | | | | | | | |
| | | brane 4 superfamlly; | | | | | | | |
| | | chrom 7 | | | | | | | |
| RC_T68873_f_at | PPAAA | | ˜−61.8 | −46.51 | 440 | A | D | −8.3 | −14.71 |
| RC_T40995_f_at | PPAAA | | −4.3 | −5.18 | 856 | A | D | −4.2 | −4.98 |
| RC_H81070_f_at | PPAAA | | ˜−60.5 | −38.36 | −183 | A | D | ˜−64.2 | −39.64 |
| RC_N30796_at | PPPAA | | −3.7 | −3.04 | 711 | A | D | −2.6 | −1.48 |
| RC_W37778_f_at | PPAAA | | −4.6 | −3.92 | 280 | A | D | −4.3 | −3.58 |
| RC_R70212_s_at | PPAAA | | −7.4 | −6.17 | 115 | A | D | −6.2 | −4.77 |
| RC_AA426330_at | PPAAA | | ˜−23.3 | −14.18 | −23 | A | D | ˜−19.4 | −20.17 |
| RC_N33927_s_at | PPAAA | | −4.1 | −2.44 | 109 | A | D | ˜−3.8 | −2.11 |
| RC_T90190_s_at | PPAAA | | −8.8 | −5.67 | 170 | A | D | −3.4 | −1.73 |
| RC_AA447145_at | PPPAA | | −6.2 | −3.44 | 130 | A | D | −2.9 | −1.04 |
| RC_H75860_at | PPAAA | | ˜−10.8 | −5.85 | 29 | A | D | ˜−8.6 | −7.11 |
| RC_T71132_s_at | PPAAA | | ˜−8.5 | −4.61 | 9 | A | D | ˜−8.6 | −4.57 |

GAINED from N to tumour
(oncogene)

| | | | | | | | | | |
|---|---|---|---|---|---|---|---|---|---|
| RC_AA609013_s_{—} | APPPP | microsomal dipepti- | 7.5 | 12.38 | 2447 | P | I | 5.8 | 8.12 |
| at | | dase (also on 6.8 k); | | | | | | | |
| | | chrom 16 | | | | | | | |
| RC_AA232508_at | APPPP | CGI-89 protein; un- | 3.1 | 2.5 | 3179 | P | I | 5.7 | 8.6 |
| | | named protein product; | | | | | | | |
| | | hypothetical protein | | | | | | | |
| RC_AA428964_at | APPPP | serine protease-like | 3.1 | 2.19 | 1037 | P | I | 4.3 | 3.56 |
| | | protease; serine pro- | | | | | | | |
| | | tease homolog = NES1; | | | | | | | |
| | | normal epithelial cell- | | | | | | | |
| | | specific 1 | | | | | | | |
| RC_T52813_s_at | APPPP | dJ28O10.2 (G0S2 | 7.3 | 8.03 | 604 | P | I | 1.4 | 0.14 |
| | | (PUTATIVE LYM- | | | | | | | |
| | | PHOCYTE G0/G1 | | | | | | | |
| | | SWITCH PROTEIN 2; | | | | | | | |
| | | chrom 1 | | | | | | | |
| RC_AA075642_at | APPPP | gp-340 variant protein; | 20.1 | 34.59 | 7085 | P | I | 9.7 | 23.8 |
| | | DMBT1/8kb.2 protein | | | | | | | |
| RC_AA007218_at | APPPP | chrom 13 no homology | 5.7 | 4.71 | 711 | P | I | ˜5.4 | 4.47 |
| RC_N33920_at | APPPP | ubiquitin-like protein | ˜12.5 | 7.18 | 524 | P | I | ˜5.8 | 3.73 |
| | | FAT10; diubiquitin; | | | | | | | |
| | | dJ271M21.6 (Diubiqui- | | | | | | | |
| | | tin); chrom 6 | | | | | | | |
| RC_N71781_at | APPPP | KIAA1199 protein, | ˜9.4 | 4.81 | 579 | P | I | ˜6.6 | 4.59 |
| | | chrom 15 | | | | | | | |
| RC_R67275_s_at | APPPP | alpha-1 (type XI) | ˜11.8 | 6.7 | 411 | P | I | ˜4.3 | 2.06 |
| | | collagen precursor; | | | | | | | |
| | | collagen, type XI, | | | | | | | |
| | | alpha 1; collagen type | | | | | | | |
| | | XI alpha-1 isoform A; | | | | | | | |
| | | chrom 1 | | | | | | | |
| RC_W80763_at | APPPP | hypothetical protein; | 5 | 3.82 | 747 | P | I | 3.9 | 2.5 |
| | | chrom 17 | | | | | | | |
| RC_AA443793_at | APPPP | chrom 7p22 | 3.7 | 2.51 | 473 | P | I | 1.4 | 0.12 |
| | | AC006028 BAC clone | | | | | | | |
| RC_AA034499_s_{—} | APPPP | ZNF198 protein; zinc | 4.3 | 2.56 | 464 | P | I | ˜3.2 | 1.47 |
| at | | finger protein; FIM | | | | | | | |
| | | protein; Cys-rich pro- | | | | | | | |
| | | tein; zinc finger protein | | | | | | | |
| | | 198; chrom 13 | | | | | | | |
| RC_AA035482_at | APPPP | chrom 5; AK022505 | 2.2 | 0.67 | 592 | P | I | 3 | 1.37 |
| | | clone; CalcineurinB | | | | | | | |
| | | (weakly similar) | | | | | | | |
| RC_AA024482_at | APPPP | hypothetical protein; | ˜21.9 | 10.07 | 443 | P | I | ˜22.7 | 11.3 |
| | | unnamed protein | | | | | | | |
| | | product; chrom 17 | | | | | | | |
| RC_H93021_at | APPPP | chrom 2; XM_004890 | 3.3 | 1.58 | 590 | P | I | 2.7 | 1.3 |
| | | peptidylprolyl isomer- | | | | | | | |
| | | ase A (cyclophilin A) | | | | | | | |
| RC_AA427737_at | APPPP | no homology | 3.5 | 1.77 | 461 | P | I | 3.2 | 1.37 |
| RC_AA417078_at | APPPP | chrom 7q31; | 3.7 | 1.71 | 337 | P | I | 2.7 | 0.83 |
| | | AF017104 clone | | | | | | | |
| M29873_s_at | APPPP | cytochrome P450-IIB | ˜38.3 | 17.75 | 400 | P | I | 5.5 | 3.08 |
| | | (hIIB3); 19q13.1- | | | | | | | |
| | | q13.2 | | | | | | | |
| RC_H27498_f_at | AAPPP | | 3.6 | 7.91 | 7876 | P | I | 3.1 | 5.35 |
| RC_T92363_s_at | AAPPP | | 3.5 | 1.18 | 311 | P | I | ˜2.4 | 0.53 |
| RC_N89910_at | AAAPP | | 1.5 | 0.12 | 250 | P | NC | −2.3 | −0.74 |
| RC_W60516_at | AAAPP | | 1.1 | 0.02 | 404 | P | NC | −2.7 | −1.51 |
| RC_AA219699_at | AAAPP | | ˜2.7 | 0.4 | 293 | P | I | ˜2.6 | 0.57 |
| RC_AA449450_at | AAAPP | | 1.7 | 0.27 | 395 | P | NC | −1.7 | −0.3 |

A classifiers
Gained in Duke A and Inc; NC in other Dukes;
Avg Diff in A > 300

| | | | | | | | | | |
|---|---|---|---|---|---|---|---|---|---|
| RC_AA599199_at | APAAA | ALU seq. | ˜5.8 | 2.11 | 568 | A | NC | −2.1 | −0.83 |
| RC_R12694_at | APAAA | unnamed protein | 1.8 | 0.22 | 213 | A | NC | ˜1.6 | 0.11 |
| | | product BAA91641, | | | | | | | |
| | | chrom 10 | | | | | | | |
| RC_H91325_s_at | APAAA | aldolase B; aldolase B | −1.3 | −0.03 | 96 | A | NC | 1 | 0 |
| | | (aa 1-364); chrom 9 | | | | | | | |
| RC_N51709_at | APAAA | chrom X | 1 | 0 | 435 | A | NC | −3 | −2.05 |
| RC_N72610_at | APAAA | — | 1.2 | 0.06 | 373 | A | NC | −3.7 | −3.23 |

Lost in Duke A and Dec; NC in other Dukes
Avg Diff in N > 300

| | | | | | | | | | |
|---|---|---|---|---|---|---|---|---|---|
| RC_N32411_f_at | PAPPP | Myc-associated zinc- | 1.2 | 0.12 | 3403 | P | NC | −1.3 | −0.27 |
| | | finger protein of hu- | | | | | | | |
| | | man islet; chrom 16 | | | | | | | |
| RC_AA243858_at | PAPPP | KIAA0882 protein | −1.2 | −0.03 | 322 | P | NC | −3.4 | −2.36 |
| RC_AA486283_at | PAPPP | ras-like protein; ras- | 1.1 | 0.02 | 5504 | P | NC | 1.1 | 0.07 |
| | | related C3 botulinum | | | | | | | |
| | | toxin substrate; | | | | | | | |
| | | dJ20J23 | | | | | | | |
| RC_AA490930_at | PAPPP | chrom 18; KIAA1468 | −2.5 | −0.86 | 175 | P | NC | −3.2 | −1.48 |
| | | protein | | | | | | | |

PPPPP; Inc in Duke A; NC in other Dukes
Avg Diff N >= 200; Fold change N to A >= 3x

| | | | | | | | | | |
|---|---|---|---|---|---|---|---|---|---|
| RC_N69263_at | PPPPP | chrom 10; AK026414 | 1.9 | 0.43 | 660 | P | NC | 1.8 | 0.35 |
| | | clone (only 108 nt | | | | | | | |
| | | hom) | | | | | | | |
| RC_T15817_f_at | PPPPP | iNOS, inducible nitric | 1.8 | 0.44 | 753 | P | NC | −1.3 | −0.11 |
| | | oxide synthase | | | | | | | |

PPPPP; Dec in Duke A; NC in other Dukes
Avg Diff N >= 200; Fold change N to A >= 3x

| | | | | | | | | | |
|---|---|---|---|---|---|---|---|---|---|
| RC_H54088_s_at | PPPPP | ribosomal protein L41 | 1.2 | 0.21 | 5372 | P | NC | 1 | 0 |
| RC_H59052_f_at | PPPPP | fungal sterol-C5- | 1.1 | 0.08 | 5216 | P | NC | 1 | 0.01 |
| | | desaturase homolog; | | | | | | | |
| | | ORF; thymosin beta-4 | | | | | | | |
| RC_R49198_s_at | PPPPP | — | −1 | 0 | 5478 | P | NC | 1.1 | 0.07 |
| RC_T73572_f_at | PPPPP | ferritin L-chain; L | 1.1 | 0.03 | 5457 | P | NC | −1 | −0.02 |
| | | apoferritin | | | | | | | |
| RC_AA477483_at | PPPPP | no matching est | −1.1 | −0.03 | 4052 | P | NC | −1.1 | −0.05 |

B classifiers
Gained in Duke B and Inc; NC in other Dukes
Avg Diff in B > 300

| | | | | | | | | | |
|---|---|---|---|---|---|---|---|---|---|
| RC_T67463_s_at | AAPAA | cathepsin O2; X; K | 1.7 | 0.19 | 275 | A | NC | ˜1.3 | 0.05 |
| RC_W94688_at | AAPAA | perilipin | −1 | 0 | 92 | A | NC | −1.5 | −0.09 |
| RC_AA126743_at | AAPAA | Z97200 PAC chrom | 1.1 | 0.02 | 310 | A | NC | −1.3 | −0.07 |
| | | 1q24; PMX1 homeo- | | | | | | | |
| | | box gene | | | | | | | |
| RC_AA236547_at | AAPAA | no homology | 1.3 | 0.05 | 191 | A | NC | −2.1 | −0.6 |
| RC_AA255567_at | AAPAA | angiopoietin-related | 1.7 | 0.2 | 235 | A | NC | −1.4 | −0.1 |
| | | protein-2; angiopoietin- | | | | | | | |
| | | like 2 | | | | | | | |
| RC_AA421256_at | AAPAA | — | −1.1 | −0.01 | 269 | A | NC | −3.8 | −2.9 |

Lost in Duke B and Dec; NC in other Dukes
Avg Diff in N > 300

| | | | | | | | | | |
|---|---|---|---|---|---|---|---|---|---|
| RC_D59847_at | PPAPP | proSAAS; granin-like | −1.9 | −0.56 | 423 | P | NC | −1.5 | −0.22 |
| | | neuroendocrine pep- | | | | | | | |
| | | tide precursor | | | | | | | |
| RC_F05038_at | PPAPP | polyamine modulated | −1 | 0 | 1068 | P | NC | −4.4 | −7.65 |
| | | factor-1; polyamine | | | | | | | |
| | | modulated factor 1 | | | | | | | |
| RC_N41059_at | PPAPP | chrom 3 | 1.1 | 0.01 | 287 | P | NC | −3.4 | −2.28 |
| RC_T23460_at | PPAPP | chrom 3; IFNAR2 | −1.3 | −0.08 | 318 | P | NC | −1.9 | −0.47 |
| | | 21q22.11 | | | | | | | |
| RC_W42789_at | PPAPP | chrom 8 AF268037 | −2.4 | −0.61 | 489 | P | NC | 1.6 | 0.21 |
| | | C8ORF4 protein | | | | | | | |
| | | (C8ORF4) chrom 8 | | | | | | | |
| | | ORF | | | | | | | |
| RC_AA460017_l_{—} | PPAPP | BAC clone chrom 16 | −2.1 | −0.85 | 952 | P | NC | −1.4 | −0.23 |
| at | | | | | | | | | |
| RC_AA482127_at | PPAPP | KIAA1142 protein | −1.7 | −0.23 | 332 | P | NC | −1.1 | −0.01 |
| RC_AA504806_at | PPAPP | chrom 2 AF052107 | −1.2 | −0.04 | 414 | P | NC | −3.5 | −2.64 |
| | | clone 23620 mRNA | | | | | | | |
| | | sequence | | | | | | | |

Inc in Duke B; NC in other Dukes
Avg Diff N >= 200; Fold change N to A >= 3x

| | | | | | | | | | |
|---|---|---|---|---|---|---|---|---|---|
| RC_AA386386_s_{—} | PPPPP | — | −1.1 | −0.02 | 1731 | P | NC | 1.1 | 0.04 |
| at | | | | | | | | | |
| RC_AA452549_at | PPPPP | PR01659; hypotheti- | 1.1 | 0.01 | 257 | P | NC | −1.6 | −0.17 |
| | | cal protein chrom 11 | | | | | | | |

Dec in Duke B; NC in other dukes
Avg Diff N >= 200; Fold change N to B >= 3x

| | | | | | | | | | |
|---|---|---|---|---|---|---|---|---|---|
| RC_T90037_at | PPPPP | unnamed protein | −2.4 | −1.07 | 466 | P | NC | −2 | −0.69 |
| | | product, chrom 4 | | | | | | | |
| RC_AA432130_at | PPPPP | KIAA0867 protein, | −1.9 | −0.56 | 437 | P | NC | −6.4 | −9.82 |
| | | chrom 12 | | | | | | | |

C classifiers
Gained in Duke C and Inc; NC in other Dukes
Avg Diff in C > 300

| | | | | | | | | | |
|---|---|---|---|---|---|---|---|---|---|
| RC_D45556_at | AAAPA | chrom 15; AL390085 | 1.4 | 0.11 | 362 | A | NC | −3 | −1.89 |
| | | clone | | | | | | | |
| RC_W86214_at | AAAPA | | 3.5 | 1.34 | 247 | A | NC | −1.4 | −0.1 |
| RC_AA039439_s_{—} | AAAPA | novel gene KIAA0134 | 4.5 | 2.81 | 259 | A | NC | ˜1.7 | 0.19 |
| at | | protein 19q13.3 | | | | | | | |
| RC_AA128935_at | AAAPA | | 2.6 | 0.84 | 412 | A | NC | ˜1.8 | 0.26 |
| RC_AA134158_s_{—} | AAAPA | class I homeodomain; | 3.9 | 2.87 | 416 | A | NC | −1.7 | −0.34 |
| at | | homeobox protein, | | | | | | | |
| | | chrom 7 | | | | | | | |
| RC_AA232646_at | AAAPA | chrom 17, AF266756 | 5 | 3.27 | −18 | A | NC | ˜1.1 | 0.01 |
| | | sphingosine kinase | | | | | | | |
| | | (SPHK1 | | | | | | | |
| RC_AA401184_at | AAAPA | no homology | 1.4 | 0.14 | 78 | A | NC | ˜0.4 | 0.63 |
| RC_AA436840_at | AAAPA | | 1.7 | 0.3 | 57 | A | NC | −1.5 | −0.08 |
| RC_AA488655_at | AAAPA | | ˜9.6 | 5.32 | 124 | A | NC | ˜4.1 | 1.01 |

L st in Duke C and Dec; NC in other Dukes
Avg Diff in N > 300

| | | | | | | | | | |
|---|---|---|---|---|---|---|---|---|---|
| RC_N30231_at | PPPAP | Lsm4 protein; U6 | ˜−59.3 | −36 | 808 | P | NC | −1.9 | −0.67 |
| | | snRNA-associated | | | | | | | |
| | | Sm-like protein LSm4; | | | | | | | |
| | | glycine-rich protein | | | | | | | |
| RC_W73790_f_at | PPPAP | immunoglobulin- | −4 | −5.39 | 989 | P | NC | −8.6 | −23.38 |
| | | related protein 14.1; | | | | | | | |
| | | lambda L-chain C | | | | | | | |
| | | region; omega protein, | | | | | | | |
| | | chrom 22 | | | | | | | |
| RC_AA412184_at | PPPAP | chrom 1p36; d89060 | −2 | −0.54 | 545 | P | NC | −1.4 | −0.13 |
| | | dolichyl- | | | | | | | |
| | | diphosphooligosaccha- | | | | | | | |
| | | ride-protein glycosyl- | | | | | | | |
| | | transferase | | | | | | | |
| RC_AA521303_at | PPPAP | methionine adenosyl- | −2.3 | −0.87 | 741 | P | NC | −1 | −0.01 |
| | | transferase regulatory | | | | | | | |
| | | beta subunit; dTDP-4- | | | | | | | |
| | | keto-6-deoxy-D- | | | | | | | |
| | | glucose 4-reductase, | | | | | | | |
| | | chrom 5 | | | | | | | |

Inc in Duke C; NC in other Dukes
Avg Diff N >= 200; Fold change N to C >= 3x

| | | | | | | | | | |
|---|---|---|---|---|---|---|---|---|---|
| RC_AA181902_at | PPPPP | AC007201 on chrom | 3.1 | 2.3 | 565 | P | NC | 1.2 | 0.04 |
| | | 19 (only 80 nt hom) | | | | | | | |

Dec in Duke C; NC in other Dukes
Avg Diff N >= 200; Fold change N to D >= 3x

| | | | | | | | | | |
|---|---|---|---|---|---|---|---|---|---|
| RC_AA461174_at | PPPPP | 8p21.3-p22 AB020860 | −3.5 | −1.3 | 170 | P | NC | −1.7 | −0.22 |
| | | anti-oncogene | | | | | | | |
| AA393432_s_at | PPPPP | chrom 2, Unknown; | −3.3 | −1.22 | 377 | P | NC | −1.3 | −0.05 |
| | | unnamed protein | | | | | | | |
| | | product AAD20029 | | | | | | | |

D classifier
Gained in Duke D and Inc; NC in other Dukes
Avg Diff in D > 300

| | | | | | | | | | |
|---|---|---|---|---|---|---|---|---|---|
| RC_N91920_at | AAAAP | chrom 16p12-p11.2; | ˜1.2 | 0.01 | 371 | P | I | ˜6.9 | 3.52 |
| | | XN_007994 retinobla- | | | | | | | |
| | | stoma binding protein | | | | | | | |
| RC_AA621601_at | AAAAP | chrom 17 XM_009868 | ˜4.2 | 0.23 | 478 | P | I | ˜12.5 | 5.32 |
| | | RAB36 ARS oncogene | | | | | | | |
| | | family | | | | | | | |

Lost in Duke D and Dec; NC in other Dukes
Avg Diff in N > 500

| | | | | | | | | | |
|---|---|---|---|---|---|---|---|---|---|
| RC_R72886_s_at | PPPPA | KIAA0422; adenylyl | −1.5 | −0.31 | 410 | A | D | −4.4 | −4.68 |
| | | cyclase type VI, chrom | | | | | | | |
| | | 12 | | | | | | | |
| RC_AA026030_at | PPPPA | chrom 1 | −1.2 | −0.06 | 514 | A | D | −2.9 | −2.02 |
| RC_Z39006_at | PPPPA | hypothetical protein, | −1.4 | −0.21 | 450 | A | D | −2.8 | −1.72 |
| | | chrom 17 | | | | | | | |
| RC_AA435908_at | PPPPA | chrom 19; ac011491 | −2 | −0.6 | 314 | A | D | −7 | −9.6 |
| | | clone and 20 nt hom. | | | | | | | |
| | | RAB2, RAS oncogene | | | | | | | |
| | | family | | | | | | | |
| RC_AA057829_s_{—} | PPPPA | growth-arrest-specific | −1 | 0 | 276 | A | D | −2.7 | −1.49 |
| at | | protein; growth arrest- | | | | | | | |
| | | specific 6; AXL stimu- | | | | | | | |
| | | latory factor, chrom 13 | | | | | | | |
| RC_R72087_at | PPPPA | chrom 5 EST; hom to | 1.1 | 0.01 | 362 | A | D | −2.5 | −1.22 |
| | | chrom 20 AL356652 | | | | | | | |
| | | clone | | | | | | | |
| RC_H04242_at | PPPPA | ras related protein | −1.5 | −0.17 | 298 | A | D | −2.3 | −0.86 |
| | | Rab5b; RAB5B, mem- | | | | | | | |
| | | ber RAS oncogene | | | | | | | |
| | | family | | | | | | | |
| RC_R97304_f_at | PPPPA | HLA-drb5; cell surface | −1.3 | −0.08 | 425 | A | D | ˜−24.3 | −16.4 |
| | | glycoprotein; MHC | | | | | | | |
| | | HLA-DR-beta chain | | | | | | | |
| | | precursor chrom 6 | | | | | | | |
| RC_N48609_at | PPPPA | chrom 11; AC004584 | 1.1 | 0.01 | 265 | A | D | −2.4 | −0.99 |
| | | chrom 17 | | | | | | | |
| RC_W86850_f_at | PPPPA | chrom 22 ? X96924 | −1.9 | −0.59 | −257 | A | D | ˜−22.8 | −14.3 |
| | | mitochondrial citrate | | | | | | | |
| | | tranbsport region | | | | | | | |
| RC_AA130603_at | PPPPA | ak024908 clone | −1.1 | −0.01 | 241 | A | D | −2.7 | −1.11 |
| RC_AA479610_at | PPPPA | singleton ak025344 | 1.1 | 0.03 | 337 | A | D | −5.3 | −6.14 |
| | | clone | | | | | | | |
| RC_AA490593_l_at | PPPPA | chrom 17 ? Synapto- | −1.4 | −0.12 | 308 | A | D | −6.5 | −7.34 |
| | | brevin2 (VAMP2) | | | | | | | |
| | | AF135372 | | | | | | | |
| RC_AA054321_s_{—} | PPPPA | 6p21 HLA class i | −1.2 | −0.03 | 63 | A | D | ˜−3.6 | −1.67 |
| at | | region; AC004202 | | | | | | | |
| | | clone | | | | | | | |

Inc in Duke D; NC in other Dukes
Avg Diff N >= 200; Fold change N to A >= 3x

| | | | | | | | | | |
|---|---|---|---|---|---|---|---|---|---|
| D79052_s_at | PPPPP | Sec61 gamma | 1.1 | 0.03 | 3411 | P | I | 3.9 | 5.13 |
| RC_T40439_s_at | PPPPP | U2 small nuclear | 1.7 | 0.39 | 2813 | P | I | 4.2 | 5.48 |
| | | ribonucleoprotein B″; | | | | | | | |
| | | dJ705D16.1 | | | | | | | |
| RC_AA251829_at | PPPPP | CGI-29 protein | 1.8 | 0.3 | 1445 | P | I | 6.7 | 7.21 |

Dec in Duke D; NC in other Dukes
Avg Diff N >= 500; Fold change N to D >= −5x

| | | | | | | | | | |
|---|---|---|---|---|---|---|---|---|---|
| RC_D60328_at | PPPPP | chrom 6, unknown; | −1.9 | −0.63 | 663 | P | D | −5.7 | −9.71 |
| | | ring finger protein 5 | | | | | | | |
| RC_H96850_at | PPPPP | oligosaccharyltransfer- | −1.3 | −0.27 | 2211 | P | D | −6.2 | −20.52 |
| | | ase d89060 1p36.1 | | | | | | | |
| | | (also C-class) | | | | | | | |
| RC_AA127444_at | PPPPP | chrom 1 no homology | −1.5 | −0.17 | 264 | P | D | −6.6 | −8.01 |
| RC_AA242824_at | PPPPP | chrom 11; ac005233 | −2.1 | −0.46 | 188 | P | D | −8.4 | −8.95 |
| | | PAC clone chrom 22 | | | | | | | |
| AA405775_s_at | PPPPP | similar to CAA16821 | −1.3 | −0.12 | 135 | P | D | −6.1 | −4.92 |
| | | (PID: g3255952) | | | | | | | |

AB classifiers
Gained in Duke AB and Inc; NC in other Dukes
Avg Diff in AB >= 300

| | | | | | | | | | |
|---|---|---|---|---|---|---|---|---|---|
| RC_AA136269_at | APPAA | HSPC314, chrom 12 | 1.8 | 0.28 | 154 | A | NC | 1.7 | 0.2 |

Lost in Duke AB and Dec; NC in other Dukes
Avg Diff in N > 300

| | | | | | | | | | |
|---|---|---|---|---|---|---|---|---|---|
| RC_T40895_at | PAAPP | protein tyrosine phos- | −1.6 | −0.34 | 1079 | P | NC | −1.1 | −0.01 |
| | | phatase PTPCAAX1; | | | | | | | |
| | | protein tyrosine phos- | | | | | | | |
| | | phatase hPRL-1N; iva- | | | | | | | |
| | | 1, chrom 6 | | | | | | | |
| RC_AA424400_at | PAAPP | chrom 14 | −1.9 | −0.35 | 166 | P | D | −5.8 | −5.07 |

PPPPP; Inc in Duke AB
Avg Diff AB >= 300; Fold change N to AB >= 5x

| | | | | | | | | | |
|---|---|---|---|---|---|---|---|---|---|
| RC_N22015_at | PPPPP | unnamed protein | 4.4 | 5.98 | 2893 | P | I | 4.5 | 6.17 |
| | | product, chrom 17; | | | | | | | |
| | | BC class. | | | | | | | |
| RC_N50971_at | PPPPP | — | 5.9 | 4.62 | 599 | P | I | 3.1 | 1.71 |
| RC_AA194833_at | PPPPP | senescence- | 3.7 | 1.78 | 1281 | P | I | 7.3 | 7.88 |
| | | associated epithelial | | | | | | | |
| | | membrane protein; | | | | | | | |
| | | Claudin-1, chrom 3 | | | | | | | |
| RC_AA393803_at | PPPPP | chrom 6 | 6.6 | 3.59 | 400 | P | I | 7.3 | 4.28 |

BC classifiers
Gained in Duke BC and Inc; NC in other Dukes
Avg Diff in BC > 300

| | | | | | | | | | |
|---|---|---|---|---|---|---|---|---|---|
| RC_AA148923_at | AAPPA | DEPP; decidual pro- | 2.8 | 1.11 | 235 | A | NC | 1.2 | 0.02 |
| | | tein induced by proges- | | | | | | | |
| | | terone; hypothetical | | | | | | | |
| | | protein, chrom 10 | | | | | | | |

Lost in Duke BC and Dec
Avg Diff in N > 300

| | | | | | | | | | |
|---|---|---|---|---|---|---|---|---|---|
| RC_N91919_s_at | PPAAP | mineralocorticoid | ˜−12.0 | −7.51 | 136 | P | D | −3.7 | −1.89 |
| | | receptor; mineralocor- | | | | | | | |
| | | ticoid receptor (al- | | | | | | | |
| | | dosterone receptor); | | | | | | | |
| | | chrom 4 | | | | | | | |
| RC_T99196_s_at | PPAAP | chrom 5 AF010235 | −2.7 | −1.04 | 221 | P | D | −2.7 | −1.13 |
| | | Homo sapiens mRNA | | | | | | | |
| | | from chromosome | | | | | | | |
| | | 5q31-33 region | | | | | | | |
| RC_W72366_at | PPAAP | chrom 1 clone RP11- | −2.8 | −0.89 | 140 | P | D | −6.4 | −5.55 |
| | | 404F10 on chromo- | | | | | | | |
| | | some 1q23.1-24.1, | | | | | | | |
| | | CD48 hom | | | | | | | |
| RC_AA255903_at | PPAAP | CD39L4; CD39-like 4; | −5.7 | −4.69 | 204 | P | MD | −11.5 | −15.73 |
| | | chrom 14 | | | | | | | |

PPPPP; Inc in Duke BC
Avg Diff N >= 300; Fold change N to BC >= 5x

| | | | | | | | | | |
|---|---|---|---|---|---|---|---|---|---|
| J03464_s_at | PPPPP | collagen alpha-2 type | 5.8 | 7.31 | 2206 | P | I | 6.3 | 8.46 |
| | | I mRNA COL2A1 | | | | | | | |
| RC_N22015_at | PPPPP | unnamed protein | 4.4 | 5.98 | 2893 | P | I | 4.5 | 6.17 |
| | | product, chrom 17 | | | | | | | |
| RC_AA055896_at | PPPPP | chrom 9 | 3.9 | 4.33 | 1553 | P | MI | 2.5 | 1.48 |
| RC_AA121315_at | PPPPP | KIAA1077 protein; | 4.8 | 5.33 | 1220 | P | NC | 3.4 | 2.57 |
| | | hypothetical protein, | | | | | | | |
| | | chrom 8 | | | | | | | |
| RC_AA122386_at | PPPPP | procollagen alpha | 3.7 | 3.51 | 1716 | P | I | 3.7 | 3.47 |
| | | 2(V); pro-alpha | | | | | | | |
| | | (V)collagen (AA 1099), | | | | | | | |
| | | chrom 2 | | | | | | | |

PPPPP; DEC in Duke BC
Avg Diff N >= 500; Fold change N to BC >= 5x

| | | | | | | | | | |
|---|---|---|---|---|---|---|---|---|---|
| C02386_s_at | PPPPP | | −5.4 | −8.64 | 969 | P | D | −3.5 | −4.58 |
| M12272_s_at | PPPPP | alcohol dehydrogen- | −25.7 | −30 | 290 | P | D | −10.6 | −16.96 |
| | | ase class I gamma | | | | | | | |
| | | subunit (ADH3) | | | | | | | |
| M12759_at | PPPPP | Ig J chain gene | −19.2 | −20.94 | 190 | P | D | −11.7 | −15.55 |
| M83670_s_at | PPPPP | carbonic anhydrase | −6.6 | −8.32 | 249 | P | D | −9.4 | −11.84 |
| | | IV | | | | | | | |
| N91087_at | PPPPP | dJ991C6.1 (novel | −5.5 | −4.68 | 350 | P | D | −2.6 | −1.32 |
| | | protein similar to C. | | | | | | | |
| | | elegans F55A12.9 | | | | | | | |
| | | (Tr: P91086)); un- | | | | | | | |
| | | named protein | | | | | | | |
| | | product; chrom 6 | | | | | | | |
| RC_H77597_f_at | PPPPP | unident. EST chrom /// | −8.7 | −18.86 | 979 | P | D | −15.4 | −49.46 |
| | | AFFY = similar metal- | | | | | | | |
| | | lothionein | | | | | | | |
| RC_N23665_s_at | PPPPP | L11708 ESTRADIOL | −14 | −11.67 | 143 | P | D | −22.4 | −28.45 |
| | | 17 BETA- | | | | | | | |
| | | DEHYDROGENASE | | | | | | | |
| | | 2, chrom 16 | | | | | | | |
| RC_N79237_at | PPPPP | unnamed protein | −15.7 | −34.48 | 499 | P | D | −40.1 | −92.94 |
| | | product, hom to | | | | | | | |
| | | S49589 cortical gran- | | | | | | | |
| | | ule lectin - African | | | | | | | |
| | | clawed frog;. | | | | | | | |
| RC_N80129_f_at | PPPPP | MT-11 protein; metal- | −24.5 | −31.69 | 737 | P | D | −14.8 | −40.79 |
| | | lothionein IX; metal- | | | | | | | |
| | | lothionein 1L, chrom | | | | | | | |
| | | 16 & chrom 1 | | | | | | | |
| RC_T24011_at | PPPPP | — | −5.7 | −4.85 | 371 | P | D | −2.8 | −1.54 |
| RC_T90492_at | PPPPP | IGJ, P01591 IMMU- | −15 | −27.44 | 623 | P | D | −21.6 | −57.11 |
| | | NOGLOBULIN J;. | | | | | | | |
| | | chrom 4 | | | | | | | |
| RC_AA058357_s_{—} | PPPPP | singleton; no hom., | −11.3 | −23.01 | 1392 | P | D | −3.7 | −6.14 |
| at | | NONSPECIFIC | | | | | | | |
| | | CROSSREACTING | | | | | | | |
| | | ANTIGEN.;. | | | | | | | |
| RC_AA133469_at | PPPPP | cytokeratin 20, chrom | −7.9 | −12.52 | 1359 | P | D | −2.6 | −2.44 |
| | | 17 | | | | | | | |
| RC_AA151674_at | PPPPP | carbonic anhydrase | −13.7 | −14.72 | 327 | P | D | −5.6 | −6.63 |
| | | XII precursor, chrom | | | | | | | |
| | | 15 | | | | | | | |
| RC_AA405715_at | PPPPP | unknown | −8 | −15.29 | 1120 | P | D | −10.4 | −32.45 |
| RC_AA428410_at | PPPPP | KIAA0828 protein, | −14.8 | −10.91 | 79 | P | D | −9.9 | −8 |
| | | chrom 7 | | | | | | | |
| RC_AA429253_at | PPPPP | KIAA0803 protein; | −5 | −3.15 | 213 | P | D | −7.3 | −8.35 |
| | | Centrosome- and | | | | | | | |
| | | Golgi-localized PKN- | | | | | | | |
| | | associated protein | | | | | | | |
| | | (CG-NAP); AKAP450, | | | | | | | |
| | | A-kinase anchoring | | | | | | | |
| | | protein AKAP350, | | | | | | | |
| | | chrom 7 | | | | | | | |
| RC_AA487468_at | PPPPP | chrom 7 | −7.2 | −5.56 | 319 | P | D | −2.6 | −1.17 |
| RC_AA621680_at | PPPPP | zinc finger transcrip- | −9.7 | −9.77 | 371 | P | D | −3.2 | −2.3 |
| | | tion factor GKLF; EZF; | | | | | | | |
| | | hEZF; endothelial | | | | | | | |
| | | Kruppel-like zinc finger | | | | | | | |
| | | protein, transforming | | | | | | | |
| | | oncogene | | | | | | | |
| Y09616_at | PPPPP | putative carboxyles- | −7 | −8.77 | 333 | P | D | −5.5 | −6.52 |
| | | terase | | | | | | | |
| AA171913_at | PPPPP | carbonic anhydrase | −51.6 | −23.46 | 148 | P | D | −7.2 | −6.84 |
| | | XII (CA12) chrom 15 | | | | | | | |
| AA253330_s_at | PPPPP | hypothetical protein; | −6.4 | −9.59 | 697 | P | D | −3.7 | −4.33 |
| | | unnamed protein | | | | | | | |
| | | product, chrom 15 | | | | | | | |

B. Finding Potential Classifier Genes for c l rectal canc r (Dukes A, B, C & D) by Sorting acc rding to Pearson Correlation co fficient

Primary Selection Criteria for Classifier Genes:

1. All genes with a score of A (AbsCall) or NC (DiffCall) for all groups (N, A, B, C & D) were removed.

2. Genes with a fold change below 5 and a Sort Score below 0.5 were removed.

3. If DiffCall were NC for a gene in a particular experiment the FC were set to 1.

Secondary Selection Criteria for Classifier Genes:

Based on Pearson correlation coefficient (FIG. 1) genes similar to a predefined profile were selected.

[image]

Classifier Genes for Dukes A, B, C and D:

TABLE III
A classifiers (Profile 1, 0, 0, 0),
Pearson correlations approach

| | |
|---|---|
| Hu6800 | |
| D87444_at | Human mRNA for KIAA0255 “gene,” |
| | complete cds |
| U18291_at | Human CDC16Hs “mRNA,” complete cds |
| L76568_xpt3_f_at | S26 from Homo sapiens excision and |
| | cross link repair protein (ERCC4) |
| | “gene,” complete genomic sequence. |
| | /gb = L76568 /ntype = DNA /annot = exon |
| U45328_s_at | “Human ubiquitin-conjugating enzyme |
| | (UBE2I)” “mRNA,” “complete cds” |
| Z14982_ma1_at | H. sapiens gene for major histocom- |
| | patibility complex encoded proteasome |
| | subunit LMP7. |
| AD000092_cds7_s_at | RAD23A gene (human RAD23A homolog) |
| | extracted from Homo sapiens DMA from |
| | chromosome 19p13.2 cosmids “R31240,” |
| | R30272 and R28549 containing the |
| | “EKLF,” “GCDH,” “CRTC,” |
| | and RAD23A “genes,” genomic sequence |
| D86973_at | Human mRNA for KIAA0219 “gene,” |
| | partial cds |
| X81636_at | H. sapiens clathrin light chain a gene |
| M59916_at | Human acid sphingomyelinase (ASM) |
| | “mRNA,” complete cds |
| X85781_s_at | “H. sapiens NOS2” “gene,” |
| | “exon 27 /gb = X85781 /ntype = DNA |
| | /annot = exon” |
| M57731_s_at | “Human gro-beta” “mRNA,” |
| | “complete cds” |
| U49188_at | Human placenta (Diff33) “mRNA,” |
| | complete cds |
| X53800_s_at | Human mRNA for macrophage |
| | inflammatory protein-2beta (MIP2beta) |
| U56816_at | Human kinase Myt1 (Myt1) “mRNA,” |
| | complete cds. |
| HG1067-HT1067_r_at | Mucin (Gb: M22406) |
| EST: | |
| RC_F03077_f | Chromosom 17, clone hRPC.15 |
| RC_AA599199 | Alu seq |
| RC_AA207015 | clone RP4-733M16 on chromosom |
| | 1p36.11-36.23 |
| RC_AA234916 | Chromosome 19 clone CTC-461H2 |
| RC_N92239_a | Wnt inhibitory factor-1 (WIF-1), |
| | chromosome 12 |
| RC_N93958_s | Phospholipase A2, group X (PLA2G10), |
| U95301_at | Phospholipase A2, group X (PLA2G10), |
| RC_AA426330 | Chromosome 17, clone hRPC.1110_E_20 |
| RC_AA024658 | clone SCb-254N2 (UWGC: rg254N02) |
| | from 6p21 |
| RC_H88540_a | heat shock protein 90, 1q21.2-q22 |

B classifiers (Profile 0, 1, 0, 0)

| | |
|---|---|
| Hu6800: | |
| U57316_at | Human GCN5 (hGCNS) “gene,” |
| | complete cds |
| X66839_at | H. sapiens MaTu MN mRNA for p54/58N |
| | protein |
| J04599_at | Human hPGI mRNA encoding bone small |
| | proteoglycan I “(biglycan),” |
| | complete cds |
| X57579_s_at | H. sapiens activin beta-A subunit |
| | (exon 2) |
| J02874_at | Human adipocyte lipid-binding |
| | “protein,” complete cds |
| M11749_at | Human Thy-1 glycoprotein “gene,” |
| | complete cds |
| U06863_at | Human follistatin-related protein |
| | precursor “mRNA,” complete cds |
| U51010_s_at | “Human nicotinamide N-methyltrans- |
| | ferase” “gene,” “exon 1 and 5′ |
| | flanking region. /gb = U51010 /ntype = DNA |
| | /annot = exon” |
| U08021_at | “Human nicotinamide N-methyltrans- |
| | ferase (NNMT)” “mRNA,” |
| | “complete cds” |
| HG3044- | “”“Fibronectin,” “Alt. Splice 1” |
| HT3742_s_at | |
| X02761_s_at | Human mRNA for fibronectin (FN precursor) |
| X02544_at | Human mRNA for alpha1-acid glycoprotein |
| | (orosomucoid) |
| M62505_at | Human C5a anaphylatoxin receptor |
| | “mRNA,” complete cds |
| J05070_at | Human type IV collagenase “mRNA,” |
| | complete cds |
| U16306_at | Human chondroitin sulfate proteoglycan |
| | versican V0 splice-variant precursor |
| | peptide “mRNA,” complete cds |
| M14218_at | Human argininosuccinate lyase “mRNA,” |
| | complete cds |
| L77567_s_at | “Homo sapiens mitochondrial citrate |
| | transport protein (CTP)” “mRNA,” |
| | “3′ end” |
| M63391_ma1_at | Human desmin gene, complete cds. |
| D13643_at | Human mRNA for KIAA0018 “gene,” |
| | complete cds |
| D79985_at | Human mRNA for KIAA0163 “gene,” |
| | complete cds |
| EST: | |
| M63262_at | 5-lipoxygenase activating protein |
| | (FLAP), 13q12 |
| R67290_at | Interleukine 14 |
| N36619_at | |
| L19161_at | Translation initiation factor 2, |
| | subunit 3″, Xp22.2-22.1 |
| RC_AA496035 | Chromosome 1? (TIGR) |
| L29217_s_at | CDC-like kinase 3 (CLK3), 15q24 |
| RC_W73194_a | Dermatoponin, 1q12-q23 |
| RC_N69507_a | Hypothetical protein PRO1847 (Alu |
| | according to TIGR) |
| RC_H15814_s | adipose most abundant gene transcript 1 |
| M84526_at | D component of complement (adipsin) |

C classifiers (Profile 0, 0, 1, 0)

| | |
|---|---|
| Hu6800: | |
| M20681_at | Human glucose transporter-like protein- |
| | III “(GLUTS),” complete cds |
| D50914_at | Human mRNA for KIAA0124 “gene,” |
| | partial cds |
| L37362_at | Homo sapiens (clone d2-115) kappa |
| | opioid receptor (OPRK1) “mRNA,” |
| | complete cds |
| X66114_ma1_at | H. sapiens g n for 2-oxoglutarat |
| | carrier protein. |
| M32053_at | Human H19 RNA “gene,” complete |
| | cds (spliced in silico) |
| Y00787_s_at | Human mRNA for MDNCF (monocyte-derived |
| | neutrophil chemotactic factor) |
| U64444_at | Human ubiquitin fusion-degradation |
| | prot in (UFD1L) “mRNA,” complete |
| | cds |
| X95325_s_at | H. sapiens mRNA for DNA binding protein |
| | A variant |
| X02419_ma1_s_at | H. sapiens uPA gene |
| X57522_at | H. sapiens RING4 cDNA |
| AB001325_at | Human AQP3 gene for aquaporine 3 (water |
| | “channel),” partail cds |
| AB002315_at | Human mRNA for KIAA0317 “gene,” |
| | complete cds. /gb = AB002315 |
| | /ntype = RNA |
| L12760_s_at | “Human phosphoenolpyruvate |
| | carboxykinase (PCK1)” “gene,” |
| | “complete cds with repeats” |
| M80899_at | Human novel protein AHNAK “mRNA,” |
| | partial sequence |
| EST: | |
| RC_AA122350 | Chromosome 8 |
| AA374109_at | spondin 2, extracellular matrix |
| | protein, chromosome 4 |
| RC_AA621755 | Transcription factor Dp-2, 3q23 |
| RC_AA442069 | sodium channel 2, 12q12 |
| RC_T40767_a | Chromosome 19 |
| RC_AA488655 | Mus? |
| RC_AA398908 | |
| RC_AA447764 | Hypothetical protein, chromosome 4 |
| RC_N69136_a | |

D classifiers (Profile 0, 0, 0, 1)

| | |
|---|---|
| X17644_s_at | Human GST1-Hs mRNA for GTP-binding |
| | protein |
| Y12812_at | H. sapiens RFXAP mRNA |
| X60486_at | H. sapiens H4/g gene for H4 histone |
| X52221_at | H. sapiens ERCC2 “gene,” exons |
| | 1 & 2 (partial) |
| L06175_at | Homo sapiens P5-1 “mRNA,” complete cds |
| Z48481_at | H. Sapiens mRNA for membrane-type |
| | matrix metalloproteinase 1 |
| X54232_at | Human mRNA for heparan sulfate |
| | proteaglycan (glypican) |
| L08010_at | “Homo sapiens reg gene” “homologue,” |
| | “complete cds” |
| L27706_at | Human chaperonin protein (Tcp20) |
| | gene complete cds |
| L15533_ma1_at | Homo sapiens pancreatits-associated |
| | protein (PAP) gene, complete cds. |
| X51408_at | Human mRNA for n-chimaerin |
| K02765_at | Human complement component C3 “mRNA,” |
| | alpha and beta “subunits,” complete cds |
| U38904_at | Human zinc finger protein C2H2-25 |
| | “mRNA,” complete cds |
| EST: | |
| RC_AA121433 | Axin, chromosome 16 |
| RC_N91920_a | RB protein binding protein, chromosome 16 |
| RC_AA621601 | GTP-binding protein Rab36, chromosome 17 |
| RC_AA454020 | NADPH quinone oxidoreductase homolog; |
| | p53 induced, chromosome 2 |
| RC_Z39652_a | APM-1 gene, chromosome 18 |

Conclusion.

As can be seen from these tables we have identified a number of genes and EST's, based on two different aporoaches, that we believe are either of importance for initiating and developing colorectal cancer, or can be used to classify the disease. These genes and EST's are subdivided into potential tumor suppressors that have a reduced level during progressi n of the diseas—or that ev n completely los their expression; potential oncogenes that increas th ir level during disease progression, or ven are gain d de novo, not being xpressed at arly stages or in normal mucosa; and finally classifiers of the disease that can be us d to identify the different Dukes stages, .g. being only expressed at a certain stage.

1. A method of determining the presence or absence of a biological condition in animal tissue
comprising collecting a sample comprising cells from the tissue and/or expression products from the cells,
assaying a first expression level of at least one gene from a first gene group, wherein the gene from the first gene group is selected from genes expressed in normal tissue cells in an amount higher than expression in biological condition cells, and
assaying a second expression level of at least one gene from a second gene group, wherein the second gene group is selected from genes expressed in a normal tissue cells in an amount lower than expression in biological condition cells,
correlating the first expression level to a standard expression level for normal tissue, and/or the second expression level to a standard expression level for biological condition cells to determine the presence or absence of a biological condition in the animal issue.

2. The method of claim 1, wherein the animal tissue is selected from epithelial tissue.

3. The method of claim 2, wherein the animal tissue is selected from epithelial tissue in the gastro-intestinal tract.

4. The method of claim 3, wherein the animal tissue is selected from epithelial tissue in colon and/or rectum.

5. The method according to claim 4, wherein the animal tissue is mucosa.

6. The method of any of the preceding claims, wherein the biological condition is an adenocarcinoma, a carcinoma, a teratoma, a sarcoma, and/or a lymphoma.

7. The method of any of the preceding claims, wherein the sample is a biopsy of the tissue.

8. The method according to any of the preceding claim 1-6, wherein the sample is a cell suspension made from the tissue.

9. The method according to any of the preceding claims, wherein the sample comprises substantially only cells from said tissue.

10. The method according to claim 9, wherein the sample comprises substantially only cells from mucosa.

11. The method according to any of the claims 3-10, wherein the gene from the first gene group is selected individually from genes comprising a sequence as identified below

| | |
|---|---|
| RC_H04768_at | chrom 15 no homology |
| RC_Z39652_at | Y14593 APM-1 gene adipocyte-specific |
| | secretory protein; chrom 1q21.3-q23 |
| RC_H30270_at | chrom 18 PAAAA in colon & bladder |
| | no homology |
| RC_T47089_s_at | tenascin-X; tenascin-X precursor; |
| | unidentified protein |
| RC_W31906_at | secretagogin; dJ501N12.8 (putative |
| | protein) chrom 6 |
| RC_AA279803_at | chrom 2 no homology |
| RC_R01646_at | chrom 13q32.1-33.3; AL159152; |
| | homology to mous Pcbp1 - |
| | poly(rC)-binding protein 1 |
| RC_AA099820_at | BAC clone AC016778 |
| AA319615_at | secretory carrier membrane protein; |
| | secretory carrier membrane protein |
| | 2; chrom 15 |
| H07011_at | tetraspan NET-6 mRNA; transmembrane |
| | 4 superfamily; chrom 7 |
| RC_T68873_f_at | |
| RC_T40995_f_at | |
| RC_H81070_f_at | |
| RC_N30796_at | |
| RC_W37778_f_at | |
| RC_R70212_s_at | |
| RC_AA426330_at | |
| RC_N33927_s_at | |
| RC_T90190_s_at | |
| RC_AA447145_at | |
| RC_H75860_at | |
| RC_T71132_s_at | |

wherein the notation refers to Accession No. in the database UniGene (Build 18).

12. The method according to claim 11, wherein the gene from the first gene group is selected individually from genes comprising a sequence as identified below

| | |
|---|---|
| RC_H04768_at | chrom 15 no homology |
| RC_Z39652_at | Y14593 APM-1 gene adipocyte-specific |
| | secretory protein; chrom 1q21.3-q23 |
| RC_H30270_at | chrom 18 PAAAA in colon & bladder |
| | no homology |
| RC_T47089_s_at | tenascin-X; tenascin-X precursor; |
| | unidentified protein |
| RC_W31906_at | secretagogin; dJ501N12.8 (putative |
| | protein) chrom 6 |
| RC_AA279803_at | chrom 2 no homology |
| RC_R01646_at | chrom 13q32.1-33.3; AL159152; |
| | homology to mouse Pcbp1 - |
| | poly(rC)-binding protein 1 |
| RC_AA099820_at | BAC clone AC016778 |
| AA319615_at | secretory carrier membrane protein; |
| | secretory carrier membrane protein |
| | 2; chrom 15 |
| H07011_at | tetraspan NET-6 mRNA; transmembrane |
| | 4 superfamily; chrom 7 |

wherein the notation refers to Accession No. in the database UniGene (Build 18).

13. The method according to claim 12, wherein the gene from the first gene group is selected individually from genes comprising a sequence as identified below

| | |
|---|---|
| RC H04768 at | chrom 15 no homology |
| RC_Z39652_at | Y14593 APM-1 gene adipocyte-specific |
| | secretory protein; chrom 1q21.3-q23 |
| RC_H30270_at | chrom 18 PAAAA in colon & bladder |
| | no homology |
| RC_T47089_s_at | tenascin-X; tenascin-X precursor; |
| | unidentified protein |
| RC_W31906_at | secretagogin; dJ501N12.8 (putative |
| | protein) chrom 6 |
| RC_AA279803_at | chrom 2 no homology |
| RC_R01646_at | chrom 13q32.1-33.3; AL159152; |
| | homology to mouse Pcbp1 - |
| | poly(rC)-binding protein 1 |
| AA319615_at | secretory carrier membrane protein; |
| | secretory carrier membrane protein |
| | 2; chrom 15 |

wherein the notation refers to Accession No. in the database UniGene (Build 18).

14. The method according to claim 13, wherein the gene from the first gene group is selected individually from genes comprising a sequence as identified below

| | |
|---|---|
| RC_T47089_s_at | tenascin-X; tenascin-X precursor; |
| | unidentified protein |
| RC_W31906_at | secretagogin; dJ501N12.8 (putative |
| | protein) chrom 6 |
| RC_AA279803_at | chrom 2 no homology |
| AA319615_at | secretory carrier membrane protein; |
| | secretory carrier membrane protein |
| | 2; chrom 15 |

wherein the notation refers to Accession No. in the database UniGene (Build 18)

15. The method according to any of claims 3-14, wherein the second gene group are selected individually from genes comprising a sequence as identified below

| | |
|---|---|
| RC_AA609013_s_at | microsomal dipeptidase (also on 6.8 |
| | k); chrom 16 |
| RC_AA232508_at | CGI-89 protein; unnamed protein |
| | product; hypothetical protein |
| RC_AA428964_at | serine protease-like protease; serine |
| | protease homolog = NES1; normal |
| | epithelial cell-specific 1 |
| RC_T52813_s_at | dJ28O10.2 (G0S2 (PUTATIVE LYMPHOCYTE |
| | G0/G1 SWITCH PROTEIN 2; chrom 1 |
| RC_AA075642_at | gp-340 variant protein; |
| | DMBT1/8kb.2 protein |
| RC_AA007218_at | chrom 13 no homology |
| RC_N33920_at | ubiquitin-like protein FAT10; |
| | diubiquitin; dJ271M21.6 (Di- |
| | ubiquitin); chrom 6 |
| RC_N71781_at | KIAA1199 protein, chrom 15 |
| RC_R67275_s_at | alpha-1 (type XI) collagen precursor, |
| | collagen, type XI, alpha 1; collagen |
| | type XI alpha-1 isoform A; chrom 1 |
| RC_W80763_at | hypothetical protein; chrom 17 |
| RC_AA443793_at | chrom 7p22 AC006028 BAC done |
| RC_AA034499_s_at | ZNF198 protein; zinc finger protein; |
| | FIM protein; Cys-rich protein; zinc |
| | finger protein 198; chrom 13 |
| RC_AA035482_at | chrom 5; AK022505 done; CalcineurinB |
| | (weakly similar) |
| RC_AA024482_at | hypothetical protein; unnamed protein |
| | product; chrom 17 |
| RC_H93021_at | chrom 2; XM_004890 peptidylprolyl |
| | isomerase A (cyclophilin A) |
| RC_AA427737_at | no homology |
| RC_AA417078_at | chrom 7q31; AF017104 done |
| M29873_s_at | cytochrome P450-IIB (hIIB3); |
| | 19q13.1-q13.2 |
| RC_H27498_f_at | |
| RC_T92363_s_at | |
| RC_N89910_at | |
| RC_W60516_at | |
| RC_AA219699_at | |
| RC_AA449450_at | |

wherein the notation refers to Accession No. in the database UniGene (Build 18).

16. The method according to any of claims 3-15, wherein the second gene group are selected individually from genes comprising a sequence as identified below

| | |
|---|---|
| RC_AA609013_s_at | microsomal dipeptidase (also on 6.8 k); |
| | chrom 16 |
| RC_AA232508_at | CGI-89 protein; unnamed protein product; |
| | hypothetical protein |
| RC_AA428964_at | serine protease-like protease; serine |
| | protease homolog = NES1; normal |
| | epithelial cell-specific 1 |
| RC_T52813_s_at | dJ28O10.2 (G0S2 (PUTATIVE |
| | LYMPHOCYTE G0/G1 SWITCH |
| | PROTEIN 2; chrom 1 |
| RC_AA075642_at | gp-340 variant protein; DMBT1/8kb.2 protein |
| RC_AA007218_at | chrom 13 no homology |
| RC_N33920_at | ubiquitin-like protein FAT10; diubiquitin; |
| | dJ271M21.6 (Diubiquitin); chrom 6 |
| RC_N71781_at | KIAA1199 protein, chrom 15 |
| RC_R67275_s_at | alpha-1 (type XI) collagen precursor; |
| | collagen, type XI, alpha 1; collagen |
| | type XI alpha-1 isoform A; chrom 1 |
| RC_W80763_at | hypothetical protein; chrom 17 |
| RC_AA443793_at | chrom 7p22 AC006028 BAC clone |
| RC_AA034499_s_at | ZNF198 protein; zinc finger protein; |
| | FIM protein; Cys-rich protein; zinc |
| | finger protein 198; chrom 13 |
| RC_AA035482_at | chrom 5; AK022505 clone; CalcineurinB |
| | (weakly similar) |
| RC_AA024482_at | hypothetical protein; unnamed protein |
| | product; chrom 17 |
| RC_H93021_at | chrom 2; XM_004890 peptidylprolyl |
| | isomerase A (cyclophilin A) |
| RC_AA427737_at | no homology |
| RC_AA417078_at | chrom 7q31; AF017104 clone |
| M29873_s_at | cytochrome P450-IIB (hIIB3); |
| | 19q13.1-q13.2 |

wherein the notation refers to Accession No. in the database UniGene (Build 18).

17. The method according to any of claims 3-14, wherein the second gene group are selected individually from genes comprising a sequence as identified below

| | |
|---|---|
| RC_AA609013_s_at | microsomal dipeptidase (also on 6.8 k); |
| | chrom 16 |
| RC_AA232508_at | CGI-89 protein; unnamed protein product; |
| | hypothetical protein |
| RC_AA428964_at | serine protease-like protease; serine |
| | protease homolog = NES1; normal |
| | epithelial cell-specific 1 |
| RC_AA075642_at | gp-340 variant protein; DMBT1/8kb.2 protein |
| RC_AA007218_at | chrom 13 no homology |
| RC_N33920_at | ubiquitin-like protein FAT10; diubiquitin; |
| | dJ271M21.6 (Diubiquitin); chrom 6 |
| RC_N71781_at | KIAA1199 protein, chrom 15 |
| RC_R67275_s_at | alpha-1 (type XI) collagen precursor; |
| | collagen, type XI, alpha 1; collagen |
| | type XI alpha-1 isoform A; chrom 1 |
| RC_W80763_at | hypothetical protein; chrom 17 |
| RC_AA034499_s_at | ZNF198 protein; zinc finger protein; FIM |
| | protein; Cys-rich protein; zinc finger |
| | protein 198; chrom 13 |
| RC_AA035482_at | chrom 5; AK022505 clone; CalcineurinB |
| | (weakly similar) |
| RC_AA024482_at | hypothetical protein; unnamed protein |
| | product; chrom 17 |
| RC_H93021_at | chrom 2; XM_004890 peptidylprolyl isomerase |
| | A (cyclophilin A) |
| RC_AA427737_at | no homology |
| RC_AA417078_at | chrom 7q31; AF017104 clone |
| M29873_s_at | cytochrome P450-IIB (hIIB3); |
| | 19q13.1-q13.2 |

wherein the notation refers to Accession No. in the database UniGene (Build 18).

18. The method according to any of claims 3-17, wherein the second gene group comprises a sequence as identified below

| | |
|---|---|
| RC_W80763_at | hypothetical protein; chrom 17 |

wherein the notation refers to Accession No. in the database UniGene (Build 18).

19. The method according to any of the preceding claims, wherein the expression level of at least two genes from the first gene group are determined.

20. The method according to any of the preceding claims, wherein the expression level of at least two genes from the second gene group are determined.

21. The method according to any of the preceding claims, further comprising the steps of determining the stage of a biological condition in the animal tissue, comprising assaying a third expression level of at least one gene from a third gene group, wherein a gene from said second gene group, in one stage, is expressed differently from a gene from said third gene group.

22. The method according to any of the preceding claims, wherein the difference in expression level of a gene from one group to the expression level of a gene from another group is at least two-fold.

23. The method according to any of the preceding claims, wherein the difference in expression level of a gene from one group to the expression level of a gene from another group is at least three-fold.

24. The method according to any of the preceding claims, wherein the expression level is determined by determining the mRNA of th cells.

25. The method according to any of the claims 1-23, wherein the expression level is determined by determining expression products, such as peptides, in the cells.

26. The method according to claim 25, wherein the expression level is determined by determining expression products, such as peptides, in the body fluids, such as blood, serum, plasma, faeces, mucus, sputum, cerebrospinal fluid, and/or urine.

27. A method of determining the stage of a biological condition in animal tissue,
comprising collecting a sample comprising cells from the tissue,
assaying the expression of at least a first stage gene from a first stage gene group and at least a second stage gene from a second stage gene group, wherein at least one of said genes is expressed in said first stage of the condition in a higher amount than in said second stage, and the other gene is a expressed in said first stage of the condition in a lower amount than in said second stage of the condition,
correlating the expression level of the assessed genes to a standard level of expression determining the stage of the condition.

28. The method according to claim 27, wherein the tissue is selected from the epithelial tissue in colon or rectum.

29. The method according to any of the preceding claims 27-28, wherein the difference in expression levels between a gene from one group to a gene from another group is at least one-fold.

30. The method according to any of the preceding claims 27-29, wherein the difference in expression levels between a gene from one group to a gene from another group is at least two-fold.

31. The method according to claim 27, wherein th stage is selected from colon cancer stag s Dukes A, Dukes B. Dukes C, and Dukes D.

32. The method according to claim 31, comprising assaying at least the expression of Dukes A stage gene from a Dukes A stage gene group, at least one Dukes B stage gene from a Dukes B stage gene group, at least the expression of Dukes C stage gene from a Dukes C stage gene group, and at least one Dukes D stage gene from a Dukes D stage gene group, wherein at least one gene from each gene group is expressed in a significantly different amount in that stage than in one of the other stages.

33. The method according to claim 32, wherein at least one gene from each gene group is expressed in a significantly higher amount in that stage than in one of the other stages.

34. The method according to claim 33, wherein a Dukes A stage gene is selected individually from any gene comprising a sequence as identified below

| | |
|---|---|
| RC_AA599199_at | ALU seq. |
| RC_R12694_at | unnamed protein product BAA91641, chrom 10 |
| RC_H91325_s_at | aldolase B; aldolase B (aa 1-364); chrom 9 |
| RC_N51709_at | chrom X |
| RC_N72610_at | — |
| RC_N69263_at | chrom 10; AK026414 clone (only 108 nt hom) |
| RC_T15817_f_at | iNOS, inducible nitric oxide synthase |

wherein the notation refers to Accession No. in the database UniGene (Build 18).

35. The method according to claim 33, wherein a Dukes B stage gene is selected individually from any gene comprising a sequence as identified below

| | | |
|---|---|---|
| RC_T67463_s_at | | cathepsin O2; X; K |
| RC_W94688_at | | perilipin |
| RC_AA126743_at | | Z97200 PAC chrom 1q24; |
| | | PMX1 homeobox gene |
| RC_AA236547_at | | no homology |
| RC_AA255567_at | | angiopoi tin-related protein-2; |
| | | angiopoietin-like 2 |
| RC_AA421256_at | | — |
| RC_AA386386_s_at | PPPPP | — |
| RC_AA452549_at | PPPPP | PR01659; hypothetical protein |
| | | chrom 11 |

wherein the notation refers to Accession No. in the database UniGene (Build 18).

36. The method according to claim 33, wherein a Dukes C stage gene is selected individually from any gene comprising a sequence as identified below

| | | |
|---|---|---|
| RC_D45556_at | | chrom 15; AL390085 clone |
| RC_W86214_at | | |
| RC_AA039439_s_at | | novel gene KIAA0134 protein 19q13.3 |
| RC_AA128935_at | | |
| RC_AA134158_s_at | | class I homeodomain; homeobox |
| | | protein, chrom 7 |
| RC_AA232646_at | | chrom 17, AF266756 sphingosine |
| | | kinase (SPHK1 |
| RC_AA401184_at | | no homology |
| RC_AA436840_at | | |
| RC_AA488655_at | | |
| RC_AA181902_at | PPPPP | AC007201 on chrom 19 |
| | | (only 80 nt hom) |

wherein the notation refers to Accession No. in the database UniGene (Build 18).

37. The method according to claim 33, wherein a Dukes D stage gene is selected individually from any gene comprising a sequence as identified below

| | | |
|---|---|---|
| RC_N91920_at | AAAAP | chrom 16p12-p11.2; XN_007994 retinoblastoma bin- |
| | | ding protein |
| RC_AA621601_at | AAAAP | chrom 17 XM_009868 RAB36 ARS oncogene family |

wherein the notation refers to Accession No. in the database UniGene (Build 18).

38. The method according to claim 32, wherein at least one gene from each gen group is expressed in a significantly lower amount in that stage than in one of the other stages.

39. The method according to claim 38, wherein a Dukes A stage gene is selected individually from any gene comprising a sequence as identified below

| | | |
|---|---|---|
| RC_N32411_f_at | PAPPP | Myc-assoclated zinc-finger protein of |
| | | human islet; chrom 16 |
| RC_AA243858_at | PAPPP | KIAA0882 protein |
| RC_AA486283_at | PAPPP | ras-like protein; ras-related C3 |
| | | botulinum toxin substrate; dJ20J23 |
| RC_AA490930_at | PAPPP | chrom 18; KIAA1468 protein |
| RC_H54088_s_at | PPPPP | ribosomal protein L41 |
| RC_H59052_f_at | PPPPP | fungal sterol-C5-desaturase homolog; |
| | | ORF; thymosin beta-4 |
| RC_R49198_s_at | PPPPP | — |
| RC_T73572_f_at | PPPPP | ferritin L-chain; L apoferritin |
| RC_AA477483_at | PPPPP | no matching est |

wherein the notation refers to Accession No. in the database UniGene (Build 18).

40. The method according to claim 38, wherein a Dukes B stage gene is selected individually from any gene comprising a sequence as identified below

| | | |
|---|---|---|
| RC_D59847_at | PPAPP | proSAAS; granin-like neuroendocrine peptide pre- |
| | | cursor |
| RC_F05038_at | PPAPP | polyamine modulated factor-1; polyamine modulated |
| | | factor 1 |
| RC_N41059_at | PPAPP | chrom 3 |
| RC_T23460_at | PPAPP | chrom3; IFNAR2 21q22.11 |
| RC_W42789_at | PPAPP | chrom 8 AF268037 C8ORF4 protein (C8ORF4) |
| | | chrom 8 ORF |
| RC_AA460017_i_at | PPAPP | BAC clone chrom 16 |
| RC_AA482127_at | PPAPP | KIAA1142 protein |
| RC_AA504806_at | PPAPP | chrom 2 AF052107 clone 23620 mRNA sequence |
| RC_T90037_at | PPPPP | unnamed protein product, chrom 4 |
| RC_AA432130_at | PPPPP | KIAA0867 protein, chrom 12 |

wherein the notation refers to Accession No. in the database UniGene (Build 18).

41. The method according to claim 38, wherein a Dukes C stage gene is selected individually from any gene comprising a sequence as identified below

| | | |
|---|---|---|
| RC_N30231_at | PPPAP | Lsm4 protein; U6 snRNA-associated |
| | | Sm-like protein LSm4; glycine-rich |
| | | protein |
| RC_W73790_f_at | PPPAP | immunoglobulin-related protein 14.1; |
| | | lambda L-chain C region; omega protein, |
| | | chrom 22 |
| RC_AA412184_at | PPPAP | chrom 1p36; d89060 dolichyl- |
| | | diphosphooligosaccharide-protein |
| | | glycosyltransferase |
| RC_AA521303_at | PPPAP | methionine adenosyltransferase |
| | | regulatory beta subunit; |
| | | dTDP-4-keto-6-deoxy-D-glucose |
| | | 4-reductase, chrom 5 |
| RC_AA461174_at | PPPPP | 8p21.3-p22 AB020860 anti-oncogene |
| AA393432_s_at | PPPPP | chrom 2, Unknown; unnamed protein |
| | | product AAD20029 |

wherein the notation refers to Accession No. in the database UniGene (Build 18).

42. The method according to claim 38, wherein a Dukes D stage gene is selected individually from any gene comprising a sequence as identified below

| | | |
|---|---|---|
| RC_R72886_s_at | PPPPA | KIAA0422; adenylyl cyclase type VI, chrom 12 |
| RC_AA026030_at | PPPPA | chrom 1 |
| RC_Z39006_at | PPPPA | hypothetical protein, chrom 17 |
| RC_AA435908_at | PPPPA | chrom 19; ac011491 clone and 20 nt hom. RAB2, |
| | | RAS oncogene family |
| RC_AA057829_s_at | PPPPA | growth-arrest-specific protein; growth arrest-specific |
| | | 6; AXL stimulatory factor, chrom 13 |
| RC_R72087_at | PPPPA | chrom 5 EST; hom to chrom 20 AL356652 clone |
| RC_H04242_at | PPPPA | ras related protein Rab5b; RAB5B, member RAS |
| | | oncogene family |
| RC_R97304_f_at | PPPPA | HLA-drb5; cell surface glycoprotein; MHC HLA-DR- |
| | | beta chain precursor chrom 6 |
| RC_N48609_at | PPPPA | chrom 11; AC004584 chrom 17 |
| RC_W86850_f_at | PPPPA | chrom 22 ? X96924 mitochondrial citrate tranbsport |
| | | region |
| RC_AA130603_at | PPPPA | ak024908 clone |
| RC_AA479610_at | PPPPA | singleton ak025344 clone |
| RC_AA490593_i_at | PPPPA | chrom 17 ? Synaptobrevin2 (VAMP2) AF135372 |
| RC_AA054321_s_at | PPPPA | 6p21 HLA class i region; AC004202 clone |
| RC_D60328_at | PPPPP | chrom 6, unknown; ring finger protein 5 |
| RC_H96850_at | PPPPP | oligosaccharyltransferase d89060 1p36.1 (also C- |
| | | class) |
| RC_AA127444_at | PPPPP | chrom 1 no homology |
| RC_AA242824_at | PPPPP | chrom 11; ac005233 PAC clone chrom 22 |
| AA405775_s_at | PPPPP | similar to CAA16821 (PID: g3255952) |

wherein the notation refers to Accession No. in the database UniGene (Build 18).

43. A method of determining an expression patt rn of a colon cell sample, comprising:
collecting sample comprising colon and/or rectum cells and/or expression products from colon and/or rectum cells,
determining the expression level of two or more genes in the sample, wherein at least one gene belongs to a first group of genes, said gene from the first gene group being expressed in a higher amount in normal tissue than in biological condition cells, and wherein at least one other gene belongs to a second group of genes, said gene from the second gene group being expressed in a lower amount in normal tissue than in biological condition cells, and the difference between the expression level of the first gene group in normal cells and biological condition cells being at least two-fold, obtaining an expression pattern of the colon and/or rectum cell sample.

44. The method of claim 43, wherein the two or more genes exclude genes which are expressed in the submucosal, muscle, or connective tissue, whereby a pattern of expression is formed for the sample which is independent of the proportion of submucosal, muscle, or connective tissue cells in the sample.

45. The method of claim 44, comprising determining the expression level of one or more genes in the sample comprising predominantly submucosal, muscle, and connective tissue cells, obtaining a second pattern, subtracting said second pattern from the expression pattern of the colon and/or rectum cell sample, forming a third pattern of expression, said third pattern of expression reflecting expression of the colorectal mucosa or colorectal cancer cells independent of the proportion of submucosal, muscle, and connective tissue cells present in the sample.

46. The method of any of the preceding claims 43-45, wherein the sample is a biopsy of the tissue.

47. The method according to any of the preceding claim 43-46, wherein the sample is a cell suspension.

48. The method according to any of the preceding claims 43-47, wherein the sample comprises substantially only cells from said tissue.

49. The method according to claim 48, wherein the sample comprises substantially only cells from mucosa.

50. The method according to any of the claims 43-47, wherein the gene from the first gene group is selected individually from

| | |
|---|---|
| RC_H04768_at | chrom 15 no homology |
| RC_Z39652_at | Y14593 APM-1 gene adipocyte-specific secretory |
| | protein; chrom 1q21.3-q23 |
| RC_H30270_at | chrom 18 PAAAA in colon & bladder no homology |
| RC_T47089_s_at | tenascin-X; tenascin-X precursor; |
| | unidentified protein |
| RC_W31906_at | secretagogin; dJ501N12.8 (putative protein) chrom 6 |
| RC_AA279803_at | chrom 2 no homology |
| RC_R01646_at | chrom 13q32.1-33.3; AL159152; homology to |
| | mouse Pcbp1 - poly(rC)-binding protein 1 |
| RC_AA099820_at | BAC clone AC016778 |
| AA319615_at | secretory carrier membrane protein; secretory carrier |
| | membrane protein 2; chrom 15 |
| H07011_at | tetraspan NET-6 mRNA; transmembrane 4 |
| | superfamily; chrom 7 |
| RC_T68873_f_at | |
| RC_T40995_f_at | |
| RC_H81070_f_at | |
| RC_N30796_at | |
| RC_W37778_f_at | |
| RC_R70212_s_at | |
| RC_AA426330_at | |
| RC_N33927_s_at | |
| RC_T90190_s_at | |
| RC_AA447145_at | |
| RC_H75860_at | |
| RC_T71132_s_at | |

wherein the notation refers to Accession No. in the database UniGene (Build 18).

51. The method according to claim 50, wherein the gene from the first gene group is selected individually from genes comprising a sequence as identified below

| | |
|---|---|
| RC_H04768_at | chrom 15 no homology |
| RC_Z39652_at | Y14593 APM-1 gene adipocyte-specific secretory |
| | protein; chrom 1q21.3-q23 |
| RC_H30270_at | chrom 18 PAAAA in colon & bladder no homology |
| RC_T47089_s_at | tenascin-X; tenascin-X precursor, |
| | unidentified protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homology to |
| | mouse Pcbp1 - poly(rC)-binding protein 1 |
| RC_AA099820_at | BAC clone AC016778 |
| AA319615_at | secretory carrier membrane protein; secretory |
| | carrier membrane protein 2; chrom 15 |
| H07011_at | tetraspan NET-6 mRNA; transmembrane 4 |
| | superfamily; chrom 7 |

wherein the notation refers to Accession No. in the database UniGene (Build 18).

52. The method according to claim 51, wherein the gene from the first gene group is selected individually from genes comprising a sequence as identified below

| | |
|---|---|
| RC_H04768_at | chrom 15 no homology |
| RC_Z39652_at | Y14593 APM-1 gene adipocyte-specific secretory |
| | protein; chrom 1q21.3-q23 |
| RC_H30270_at | chrom 18 PAAAA in colon & bladder no homology |
| RC_T47089_s_at | tenascin-X; tenascin-X precursor; |
| | unidentified protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homology to |
| | mouse Pcbp1 - poly(rC)-binding protein 1 |
| AA319615_at | secretory carrier membrane protein; secretory |
| | carrier membrane protein 2; chrom 15 |

wherein the notation refers to Accession No. in the database UniGene (Build 18).

53. The method according to claim 52, wherein the gene from the first gene group is selected individually from genes comprising a sequence as identified below

| | |
|---|---|
| RC_T47089_s_at | tenascin-X; tenascin-X precursor |
| | unidentified protein |
| RC_W31906_at | secretagogin; dJ501N12.8 (putative protein) chrom 6 |
| RC_AA279803_at | chrom 2 no homology |
| AA319615_at | secretory carrier membrane protein; secretory |
| | carrier membrane protein 2; chrom 15 |

wherein the notation refers to Accession No. in the database UniGene (Build 18).

54. The method according to any of claims 3-14, wherein the second gene group are selected individually from genes comprising a sequence as identified below

| | |
|---|---|
| RC_AA609013_s_at | microsomal dipeptidase (also on 6.8 k); |
| | chrom 16 |
| RC_AA232508_at | CGI-89 protein; unnamed protein product |
| | hypothetical protein |
| RC_AA428964_at | serine protease-like protease; serine |
| | protease homolog = NES1; normal |
| | epithelial cell-specific 1 |
| RC_T52813_s_at | dJ28O10.2 (G0S2 (PUTATIVE LYMPHOCYTE |
| | G0/G1 SWITCH PROTEIN 2; chrom 1 |
| RC_AA075642_at | gp-340 variant protein; DMBT1/8kb.2 |
| | protein |
| RC_AA007218_at | chrom 13 no homology |
| RC_N33920_at | ubiquitin-like protein FAT10; |
| | diubiquitin; dJ271M21.6 (Diubiquitin); |
| | chrom 6 |
| RC_N71781_at | KIAA1199 protein, chrom 15 |
| RC_R67275_s_at | alpha-1 (type XI) collagen precursor, |
| | collagen, type XI, alpha 1; collagen |
| | type XI alpha-1 Isoform A; chrom 1 |
| RC_W80763_at | hypothetical protein; chrom 17 |
| RC_AA443793_at | chrom 7p22 AC006028 BAC clone |
| RC_AA034499_s_at | ZNF198 protein; zinc finger protein; |
| | FIM protein; Cys-rich protein; zinc |
| | finger protein 198; chrom 13 |
| RC_AA035482_at | chrom 5; AK022505 clone; CalcineurinB |
| | (weakly similar) |
| RC_AA024482_at | hypothetical protein; unnamed protein |
| | product; chrom 17 |
| RC_H93021_at | chrom 2; XM_004890 p ptidylprolyl |
| | isomerase A (cyclophilin A) |
| RC_AA427737_at | no homology |
| RC_AA417078_at | chrom 7q31; AF017104 clone |
| M29873_s_at | cytochrome P450-IIB (hIIB3); |
| | 19q13.1-q13.2 |
| RC_H27498_f_at | |
| RC_T92363_s_at | |
| RC_N89910_at | |
| RC_W60516_at | |
| RC_AA219699_at | |
| RC_AA449450_at | |

wherein the notation refers to Accession No. in the database UniGene (Build 18).

55. The method according to any of claims 43-49, wherein the second gene group are selected individually from genes comprising a sequence as identified below

| | |
|---|---|
| RC_AA609013_s_at | microsomal dipeptidase (also on 6.8 k); |
| | chrom 16 |
| RC_AA232508_at | CGI-89 protein; unnamed protein product; |
| | hypothetical protein |
| RC_AA428964_at | serine protease-like protease; serine |
| | protease homolog = NES1; normal |
| | epithelial cell-specific 1 |
| RC_T52813_s_at | dJ28O10.2 (G0S2 (PUTATIVE LYMPHOCYTE |
| | G0/G1 SWITCH PROTEIN 2; chrom 1 |
| RC_AA075642_at | gp-340 variant protein; DMBT1/8kb.2 |
| | protein |
| RC_AA007218_at | chrom 13 no homology |
| RC_N33920_at | ubiquitin-like protein FAT10; |
| | diubiquitin; dJ271M21.6 (Diubiquitin); |
| | chrom 6 |
| RC_N71781_at | KIAA1199 protein, chrom 15 |
| RC_R67275_s_at | alpha-1 (type XI) collagen precursor; |
| | collagen, type XI, alpha 1; collagen |
| | type XI alpha-1 isoform A; chrom 1 |
| RC_W80763_at | hypothetical protein; chrom 17 |
| RC_AA443793_at | Chrom 7p22 AC006028 BAC clone |
| RC_AA034499_s_at | ZNF198 protein; zinc finger protein; |
| | FIM protein; Cys-rich protein; zinc |
| | finger protein 198; chrom 13 |
| RC_AA035482_at | chrom 5; AK022505 clone; CalcineurinB |
| | (weakly similar) |
| RC_AA024482_at | hypothetical protein; unnamed prot in |
| | product; chrom 17 |
| RC_H93021_at | chrom 2; XM_004890 peptidylprolyl |
| | isomerase A (cyclophilin A) |
| RC_AA427737_at | no homology |
| RC_AA417078_at | chrom 7q31; AF017104 clone |
| M29873_s_at | cytochrome P450-IIB (hIIB3); |
| | 19q13.1-q13.2 |

wherein the notation refers to Accession No. in the database UniGene (Build 18).

56. The method according to any of claims 43-49, wherein the second gene group are selected individually from genes comprising a sequence as identified below

| | |
|---|---|
| RC_AA609013_s_at | microsomal dipeptidase (also on 6.8 k); |
| | chrom 16 |
| RC_AA232508_at | CGI-89 protein; unnamed protein product; |
| | hypothetical protein |
| RC_AA428964_at | serine protease-like protease; serine |
| | protease homolog = NES1; normal |
| | epithelial cell-specific 1 |
| RC_AA075642_at | gp-340 variant protein; DMBT1/8kb.2 protein |
| RC_AA007218_at | chrom 13 no homology |
| RC_N33920_at | ubiquitin-like protein FAT10; diubiquitin; |
| | dJ271M21.6 (Diubiquitin); chrom 6 |
| RC_N71781_at | KIAA1199 protein, chrom 15 |
| RC_R67275_s_at | alpha-1 (type XI) collagen precursor; |
| | collagen, type XI, alpha 1; collagen type XI |
| | alpha-1 isoform A; chrom 1 |
| RC_W80763_at | hypothetical protein; chrom 17 |
| RC_AA034499_s_at | ZNF198 protein; zinc finger protein; |
| | FIM protein; Cys-rich protein; zinc finger |
| | protein 198; chrom 13 |
| RC_AA035482_at | chrom 5; AK022505 clone; CalcineurinB |
| | (weakly similar) |
| RC_AA024482_at | hypothetical protein; unnamed protein |
| | product; chrom 17 |
| RC_H93021_at | chrom 2; XM_004890 peptidylprolyl |
| | Isomerase A (cyclophilin A) |
| RC_AA427737_at | no homology |
| RC_AA417078_at | chrom 7q31; AF017104 clone |
| M29873_s_at | cytochrome P450-IIB (hIIB3); |
| | 19q13.1-q13.2 |

wherein the notation refers to Accession No. in the database UniGene (Build 18).

57. The method according to any of claims 43-49, wherein the second gene group comprises a sequence as identified below

| | |
|---|---|
| RC_W80763_at | Hypothetical protein; chrom 17 |

the notation refers to Accession No in the database UniGene (Build 18).

58. The method according to any of the preceding claims 43-57, wherein the expression level of at least two genes from the first gene group are determined.

59. The method according to any of the preceding claims 43-58, wherein the expression level of at least two genes from the second gene group are determined.

60. A method of determining an expression pattern of a colon cell sample independent of the proportion of submucosal, muscle, or connective tissue cells present, comprising:
determining the expression of one or more genes in a sample comprising cells, wherein the one or more genes exclude genes which are expressed in the submucosal, muscle, or connective tissue, whereby a pattern of expression is formed for the sample which is independent of the proportion of submucosal, muscle, or connective tissue cells in the sample.

61. The method according to claim 60, comprising determining the expression level of one or more genes in the sample comprising predominantly submucosal, muscle, and connective tissue cells, obtaining a second pattern, subtracting said second pattern from the expression pattern of the colon and/or rectum cell sample, forming a third pattern of expression, said third pattern of expression reflecting xpression of the colon cells independent of th proportion of submucosal, muscle, and connective tissue cells present in the sample.

62. A method of determining the presence or absence of a biological condition in human colon and/or rectum tissue comprising,
collecting a sample comprising cells from the tissue,
determining an expression pattern of the cells as defined in any of claims 43-61,
correlating the determined expression pattern to a standard pattern,
determining the presence or absence of the biological condition of said tissue.

63. A method for determining the stage of a biological condition in animal tissue, comprising
collecting a sample comprising cells from the tissue,
determining an expression pattern of the cells as defined in any of claims 43-61,
correlating the determined expression pattern to a standard pattern,
determining the stage of the biological condition is said tissue.

64. A method for reducing cell tumorigenicity of a cell, said method comprising
contacting a tumor cell with at least one peptide expressed by at least one gene selected from genes being expressed in an at least two-fold higher in normal cells than the amount expressed in said tumor cell.

65. The method according to claim 64, wherein the at least one gene is selected individually from genes comprising a sequence as identified below

| | |
|---|---|
| RC_H04768_at | chrom 15 no homology |
| RC_Z39652_at | Y14593 APM-1 gene adipocyte-specific secretory |
| | protein; chrom 1q21.3-q23 |
| RC_H30270_at | chrom 18 PAAAA in colon & bladder no homology |
| RC_T47089_s_at | tenascin-X; tenascin-X precursor; unidenti- |
| | fied protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homology to |
| | mouse Pcbp1 - poly(rC)-binding protein 1 |
| AA319615_at | secretory carrier membrane protein; secretory |
| | carrier membrane protein 2; chrom 15 |

wherein the notation refers to Accession No. in the database UniGene (Build 18).

66. The method according to claim 64 or 65, wherein the tumor cell is contacted with at least two different peptides.

67. A method for reducing cell tumorigenicity of a cell, said method comprising
obtaining at least one gene selected from genes being expressed in an at least twofold higher in normal cells than the amount expressed in said tumor cell,
introducing said at least one gene into the tumor cell in a manner allowing expression of said gene(s).

68. The method according to claim 67, where the at least one gene is selected individually from genes comprising a sequence as identified below

| | |
|---|---|
| RC_H04768_at | chrom 15 no homology |
| RC_Z39652_at | Y14593 APM-1 gene adipocyte-specific secretory |
| | protein; chrom 1q21.3-q23 |
| RC_H30270_at | chrom 18 PAAAA in colon & bladder no homology |
| RC_T47089_s_at | tenascin-X; tenascin-X precursor; unidentified |
| | protein |
| RC_W31906_at | secretagogin; dJ501N12.8 (putative protein) |
| | chrom 6 |
| RC_AA279803_at | chrom 2 no homology |
| RC_R01646_at | chrom 13q32.1-33.3; AL159152; homology to |
| | mouse Pcbp1 - poly(rC)-binding protein 1 |
| AA319615_at | secretory carrier membrane protein; secretory |
| | carrier membrane protein 2; chrom 15 |

wherein the notation refers to Accession No. in the database UniGene (Build 18).

69. The method according to claim 67 or 68, wherein at least two different genes are introduced into the tumor cell.

70. A method for reducing cell tumorigenicity of a cell, said method comprising
obtaining at least one nucleotide probe capable of hybridising with at least one gene of a tumor cell, said at least one gene being selected from genes being expressed in an amount at least one-fold lower in normal cells than the amount expressed in said tumor cell, and
introducing said at least one nucleotide probe into the tumor cell in a manner allowing the probe to hybridise to the at least one gene, thereby inhibiting expression of said at least one gene.

71. The method according to claim 70, wherein the nucleotide probe is selected from probes capable of hybridising to a nucleotide sequence comprising a sequence as identified below

| | | |
|---|---|---|
| RC_AA609013_s_at | APPPP | microsomal dipeptidase (also |
| | | on 6.8 k); chrom 16 |
| RC_AA232508_at | APPPP | CGI-89 protein; unnamed protein |
| | | product; hypothetical protein |
| RC_AA428964_at | APPPP | serine protease-like protease; |
| | | serine protease homolog = |
| | | NES1; normal epithelial cell- |
| | | specific 1 |
| RC_T52813_s_at | APPPP | dJ28O10.2 (G0S2 (PUTATIVE |
| | | LYMPHOCYTE G0/G1 SWITCH |
| | | PROTEIN 2; chrom 1 |
| RC_AA075642_at | APPPP | gp-340 variant protein; |
| | | DMBT1/8kb.2 protein |
| RC_AA007218_at | APPPP | chrom 13 no homology |
| RC_N33920_at | APPPP | ubiquitin-like protein FAT10; |
| | | diubiquitin; dJ271M21.6 |
| | | (Diubiquitin); chrom 6 |
| RC_N71781_at | APPPP | KIAA1199 protein, chrom 15 |
| RC_R67275_s_at | APPPP | alpha-1 (type XI) collagen |
| | | precursor; collagen, type |
| | | XI, alpha 1; collagen type |
| | | XI alpha-1 isoform A; |
| | | chrom 1 |
| RC_W80763_at | APPPP | hypothetical protein; chrom 17 |
| RC_AA443793_at | APPPP | chrom 7p22 AC006028 BAC clone |
| RC_AA034499_s_at | APPPP | ZNF198 protein; zinc finger |
| | | protein; FIM protein; |
| | | Cys-rich protein; zinc finger |
| | | protein 198; chrom 13 |
| RC_AA035482_at | APPPP | chrom 5; AK022505 clone; |
| | | CalcineurinB (weakly |
| | | similar) |
| RC_AA024482_at | APPPP | hypothetical protein; unnamed |
| | | protein product; chrom 17 |
| RC_H93021_at | APPPP | chrom 2; XM_004890 peptidyl- |
| | | prolyl isomerase A |
| | | (cyclophilin A) |
| RC_AA427737_at | APPPP | no homology |
| RC_AA417078_at | APPPP | chrom 7q31; AF017104 clone |
| M29873_s_at | APPPP | cytochrome P450-IIB (hIIB3); |
| | | 19q13.1-q13.2 |
| RC_H27498_f_at | AAPPP | |
| RC_T92363_s_at | AAPPP | |
| RC_N89910_at | AAAPP | |
| RC_W60516_at | AAAPP | |
| RC_AA219699_at | AAAPP | |
| RC_AA449450_at | AAAPP | |

wherein the notation refers to Accession No. in the database UniGene (Build 18).

72. The method according to claim 70 or 71, wherein at least two different genes ar introduced into the tumor cell.

73. A method for producing antibodies against an expression product of a cell from a biological tissue, said method comprising the steps of
obtaining expression product(s) from at least one gene said gene being expressed as defined in any of claims 27-37,
immunising a mammal with said expression product(s) obtaining antibodies against the expression product.

74. A pharmaceutical composition for the treatment of a biological condition comprising at least one antibody produced as described in claim 73.

75. A vaccine for the prophylaxis or treatment of a biological condition comprising at least one expression product from at least one gene said gene being expressed as defined in any of claims 27-37.

76. The use of a method as defined in any of claims 1-63 for producing an assay for diagnosing a biological condition in animal tissue.

77. The use of a peptide as defined in any of claims 64-66 for preparation of a pharmaceutical composition for the treatment of a biological condition in animal tissue.

78. The use of a gene as defined in any of claims 67-69 for preparation of a pharmaceutical composition for the treatment of a biological condition in animal tissue.

79. The use of a probe as defined in any of claims 70-72 for preparation of a pharmaceutical composition for the treatment of a biological condition in animal tissue.

80. An assay for determining the presence or absence of a biological condition in animal tissue, comprising
at least one first marker capable of detecting a first expression level of at least one gene from a first gene group, wherein the gene from the first gene group is selected from genes expressed in normal tissue cells in an amount higher than expression in biological condition cells,
at least one second marker capable of detecting a second expression level of at least one gene from a second gene group, wherein the second g ne group is selected from genes expressed in normal tissue cells in an amount lower than expression in biological condition cells.

81. The assay according to claim 80, wherein the marker is a nucleotide probe.

82. The assay according to claim 80, wherein the marker is an antibody.

83. The assay according to claim 80, wherein the genes are as defined in any of claims 11-18, 34-37, and 39-42.

84. An assay for determining an expression pattern of a colon and/or rectum cell, comprising at least a first marker and a second marker, wherein the first marker is capable of detecting a gene from a first gene group as defined in claim 43, and the second marker is capable of detecting a gene from a second gene group as defined in claim 43.

85. The assay according to claim 84, wherein the first marker is capable of detecting one gene as identified in Table I, and the second marker is capable of detecting another gene as identified in Table I.

86. The assay according to claim 85, comprising at least two markers for each gene group,
correlating the first expression level and the second expression level to a standard level of the assessed genes to determine the presence or absence of a biological condition in the animal tissue.

87. The assay according to claim 86, wherein the marker is a nucleotide probe

88. The assay according to claim 86, wherein the marker is an antibody.

89. A method for identifying a tissue sample as colorectal, comprising subjecting the tissue to a method as identified in any of claims 43-51, determining expression patterns and comparing the expression patterns determin d with expression patterns from colo-rectal tissue.
